(19) United States
(12) Patent Application Publication
(10) Pub. No.: US 2003/0198958 A1
(43) Pub. Date: Oct. 23, 2003

(54) Noval human proteins, polynucleotides encoding them and methods of using the same

(76) Inventors: Richard A. Shimkets, Guilford, CT (US); Elma Fernandes, Branford, CT (US); John L. Herrmann, Guilford, CT (US); Xiaohong Liu, Lexington, MA (US); Meijia Yang, East Lyme, CT (US); Ferenc L. Boldog, North Haven, CT (US); Glennda Smithson, Guilford, CT (US); Luca Rastelli, Guilford, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/098,871

(22) Filed: Mar. 13, 2002

Related U.S. Application Data
(63) Continuation-in-part of application No. 09/659,634, filed on Sep. 12, 2000.
(60) Provisional application No. 60/153,629, filed on Sep. 13, 1999.
(60) Provisional application No. 60/154,520, filed on Sep. 16, 1999.
(60) Provisional application No. 60/154,762, filed on Sep. 20, 1999.
(60) Provisional application No. 60/159,231, filed on Oct. 13, 1999.
(60) Provisional application No. 60/276,960, filed on Mar. 19, 2001.

(51) Int. Cl. C12Q 1/68; G01N 33/53; G01N 33/567; C07H 21/04; C12P 21/02; C12N 5/06; C07K 14/705; A61K 48/00; A61K 38/17

(52) U.S. Cl. 435/6; 435/7.2; 435/69.1; 435/320.1; 435/325; 514/12; 514/44; 530/350; 536/23.5

(57) ABSTRACT

The invention provides polypeptides, designated herein as POLYX polypeptides, as well as polynucleotides encoding POLYX polypeptides, and antibodies that immunospecifically-bind to POLYX polypeptide or polynucleotide, or derivatives, variants, mutants, or fragments thereof. The invention additionally provides methods in which the POLYX polypeptide, polynucleotide, and antibody are used in the detection, prevention, and treatment of a broad range of pathological states.

### RELATED APPLICATIONS

This application is a continuation-in-part of U.S. Ser. No. 09/659,634, filed Sep. 12, 2000, which claims priority to U.S. Ser. No. 60/153,629, filed Sep. 13, 1999, U.S. Ser. No. 60/154,520, filed Sep. 16, 1999, U.S. Ser. No. 60/154,762, filed Sep. 20, 1999, and U.S. Ser. No. 60/159,231, filed Oct. 31, 1999. This application also claims priority to U.S. Ser. No. 60/276,960, filed Mar. 19, 2001. The contents of these applications are incorporated by reference in their entirety.

### FIELD OF THE INVENTION

The invention relates to polynucleotides and the polypeptides encoded by such polynucleotides, as well as vectors, host cells, antibodies and recombinant methods for producing the polypeptides and polynucleotides, as well as methods for using the same.

### BACKGROUND OF THE INVENTION

Eukaryotic cells are subdivided by membranes into multiple functionally distinct compartments called organelles. Each organelle includes proteins essential for its proper function. These proteins can include sequence motifs often referred to as sorting signals. The sorting signals can aid in targeting the proteins to their appropriate cellular organelle. In addition, sorting signals can direct some proteins to be exported, or secreted, from the cell.

One type of sorting signal is a signal sequence, which is also referred to as a signal peptide or leader sequence. The signal sequence is present as an amino-terminal extension on a newly synthesized polypeptide chain. A signal sequence can target proteins to an intracellular organelle called the endoplasmic reticulum (ER).

The signal sequence takes part in an array of protein-protein and protein-lipid interactions that result in translocation of a polypeptide containing the signal sequence through a channel in the ER. After translocation, a membrane-bound enzyme, named a signal peptidase, liberates the mature protein from the signal sequence.

The ER functions to separate membrane-bound proteins and secreted proteins from proteins that remain in the cytoplasm. Once targeted to the ER, both secreted and membrane-bound proteins can be further distributed to another cellular organelle called the Golgi apparatus. The Golgi directs the proteins to other cellular organelles such as vesicles, lysosomes, the plasma membrane, mitochondria and microbodies.

Only a limited number of genes encoding human membrane-bound and secreted proteins have been identified. Examples of known secreted proteins include human insulin, interferon, interleukins, transforming growth factor-beta, human growth hormone, erythropoietin, and lymphokines.

### SUMMARY OF THE INVENTION

The invention is based, in part, upon the discovery of novel nucleic acids and secreted polypeptides encoded thereby. The nucleic acids and polypeptides are collectively referred to herein as “POLYX” nucleic acids and polypeptides.

Accordingly, in one aspect, the invention includes an isolated nucleic acid that encodes a POLYX polypeptide, or a fragment, homolog, analog or derivative thereof. For example, the nucleic acid can encode a polypeptide at least 85% identical to a polypeptide comprising the amino acid sequences of SEQ ID NO:2, 4, 6, 8, 10, 12, 14, 16, 18, 20, 22, 24, or 26. The nucleic acid can be, e.g., a genomic DNA fragment or a cDNA molecule. In some embodiments, the invention provides an isolated nucleic acid molecule that includes the nucleic acid sequence of any of SEQ ID NO:1, 3, 5, 7, 9, 11, 13, 15, 17, 19, 21, 23, or 25.

Also included within the scope of the invention is a vector containing one or more of the nucleic acids described herein, and a cell containing the vectors or nucleic acids described herein.

The invention is also directed to host cells transformed with a vector comprising any of the nucleic acid molecules described above.

In another aspect, the invention includes a pharmaceutical composition that includes a POLYX nucleic acid and a pharmaceutically acceptable carrier or diluent.

In a further aspect, the invention includes a substantially purified POLYX polypeptide, e.g., any of the POLYX polypeptides encoded by a POLYX nucleic acid, and fragments, homologs, analogs, and derivatives thereof. The invention also includes a pharmaceutical composition that includes a POLYX polypeptide and a pharmaceutically acceptable carrier or diluent.

In a still a further aspect, the invention provides an antibody that binds specifically to a POLYX polypeptide. The antibody can be, e.g., a monoclonal or polyclonal antibody, and fragments, homologs, analogs, and derivatives thereof. The invention also includes a pharmaceutical composition including POLYX antibody and a pharmaceutically acceptable carrier or diluent. The invention is also directed to isolated antibodies that bind to an epitope on a polypeptide encoded by any of the nucleic acid molecules described above.

The invention also includes kits comprising any of the pharmaceutical compositions described above.

The invention further provides a method for producing a POLYX polypeptide by providing a cell containing a POLYX nucleic acid, e.g., a vector that includes a POLYX nucleic acid, and culturing the cell under conditions sufficient to express the POLYX polypeptide encoded by the nucleic acid. The expressed POLYX polypeptide is then recovered from the cell. Preferably, the cell produces little or no endogenous POLYX polypeptide. The cell can be, e.g., a prokaryotic cell or eukaryotic cell.

The invention is also directed to methods of identifying a POLYX polypeptide or nucleic acids in a sample by contacting the sample with a compound that specifically binds to the polypeptide or nucleic acid, and detecting complex formation, if present.

The invention further provides methods of identifying a compound that modulates the activity of a POLYX polypeptide by contacting a POLYX polypeptide with a compound and determining whether the POLYX polypeptide activity is modified.

The invention is also directed to compounds that modulate POLYX polypeptide activity identified by contacting a POLYX polypeptide with the compound and determining whether the compound modifies activity of the POLYX polypeptide, binds to the POLYX polypeptide, or binds to a nucleic acid molecule encoding a POLYX polypeptide.

In a another aspect, the invention provides a method of determining the presence of, or predisposition to a POLYX-associated disorder in a subject. The method includes providing a sample from the subject and measuring the amount of POLYX polypeptide in the subject sample. The amount of POLYX polypeptide in the subject sample is then compared to the amount of POLYX polypeptide in a control sample. An alteration in the amount of POLYX polypeptide in the subject protein sample relative to the amount of POLYX polypeptide in the control protein sample indicates the subject has a tissue proliferation-associated condition. A control sample is preferably taken from a matched individual, i.e., an individual of similar age, sex, or other general condition but who is not suspected of having a tissue proliferation-associated condition. Alternatively, the control sample may be taken from the subject at a time when the subject is not suspected of having a tissue proliferation-associated disorder. In some embodiments, the POLYX is detected using a POLYX antibody.

In a further aspect, the invention provides a method of determining the presence of, or predisposition to, a POLYX-associated disorder in a subject. The method includes providing a nucleic acid sample (e.g., RNA or DNA, or both) from the subject and measuring the amount of the POLYX nucleic acid in the subject nucleic acid sample. The amount of POLYX nucleic acid sample in the subject nucleic acid is then compared to the amount of POLYX nucleic acid in a control sample. An alteration in the amount of POLYX nucleic acid in the sample relative to the amount of POLYX in the control sample indicates the subject has a tissue proliferation-associated disorder.

In a still further aspect, the invention provides a method of treating or preventing or delaying a POLYX-associated disorder. The method includes administering to a subject in which such treatment or prevention or delay is desired a POLYX nucleic acid, a POLYX polypeptide, or a POLYX antibody in an amount sufficient to treat, prevent, or delay a tissue proliferation-associated disorder in the subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting.

Other features and advantages of the invention will be apparent from the following detailed description and claims.

### BRIEF DESCRIPTION OF THE FIGURES

FIG. 1: depicts the alignment of the proteins of clones 23208248 and 23208248.0.27.

FIG. 2 is a western blot of the protein of the extracellular domain of clone 10129612-1.

FIG. 3 is a real time quantitative gene expression analysis for clone 10129612_{—}1 using probe set Ag 47 in various cell samples.

FIG. 4 is a real time quantitative gene expression analysis for clone 10129612_{—}1 using probe set Ag 47 in various surgical tissue samples.

FIG. 5 is a real time quantitative gene expression analysis for clone 10129612_{—}1 using probe set Ag 47b in various cell samples.

FIG. 6 is a real time quantitative gene expression analysis for clone 10168180.0.35 using probe set Ag121 in various cell samples.

FIG. 7 is a real time quantitative gene expression analysis for clone 10354784.0.148 using probe set Ag91 in various cell samples.

FIG. 8 is a real time quantitative gene expression analysis for clone 16532807.0.137 using probe set Ag122 in various cell samples.

FIG. 9 is a real time quantitative gene expression analysis for clone 17941787.0.3 using probe set Ag96 in various cell samples.

FIG. 10 is a real time quantitative gene expression analysis for clone 21636818.0.57 using probe set Ag96 in various cell samples.

FIG. 11 depicts a western blot of clone 10354784 polypeptide secreted by 293 cells.

FIG. 12 depicts a western blot of clone 17883252 polypeptide expressed in 293 cells.

FIG. 13 depicts a western blot of clone 17941787 polypeptide expressed by 293 cells.

### DETAILED DESCRIPTION OF THE INVENTION

The invention provides novel polynucleotides and the polypeptides encoded thereby. The invention is based in part on the discovery of nucleic acids encoding 13 proteins that contain sequences suggesting they are secreted, localized to a cellular organelle, or membrane associated. The invention includes 13 POLYX nucleic acids, POLYX polypeptides, POLYX antibodies, or compounds or methods based on these nucleic acids. These sequences are collectively referred to as “POLYX nucleic acids or “POLYX polynucleotides” and the corresponding encoded polypeptide is referred to as a “POLYX polypeptide” or “POLYX protein”.

Table 1 provides a cross-reference between a POLYX nucleic acid or polypeptide of the invention, a table disclosing a nucleic acid and encoded polypeptide that is encompassed by an indicated POLYX nucleic acid or polypeptide of the invention, and a corresponding sequence identification number (SEQ ID NO:). Also provided is a Clone Identification Number for the disclosed nucleic acid and encoded polypeptides. Unless indicated otherwise, reference to a “Clone” herein refers to a discrete in silico nucleic acid sequence.

TABLE 1

| | POLYX | Table | SEQ ID NO: | SEQ ID NO: |
|---|---|---|---|---|
| Clone | Number | Number | Nucleic Acid | Polypeptide |
| 23208248 | 1 | 2 | 1 | 2 |
| 23208248.0.27 | 2 | 3 | 3 | 4 |
| 29200321 | 3 | 4 | 5 | 6 |
| 10129612_1 | 4 | 5 | 7 | 8 |
| 10168180.0.35 | 5 | 6 | 9 | 10 |
| 10354784.0.148 | 6 | 7 | 11 | 12 |
| 13043743.0.15 | 7 | 8 | 13 | 14 |
| 16532807.0.137 | 8 | 9 | 15 | 16 |
| 17883252.0.13 | 9 | 10 | 17 | 18 |
| 17941787.0.3 | 10 | 11 | 19 | 20 |
| 20936375.0.104 | 11 | 12 | 21 | 22 |
| 21636818.0.57 | 12 | 13 | 23 | 24 |
| 20468752-018_update | 13 | 14 | 25 | 26 |

POLYX nucleic acids, POLYX polypeptides, POLYX antibodies, and related compounds, are useful in a variety of applications and contexts. For example, various POLYX nucleic acids and polypeptides according to the invention are useful, inter alia, as novel members of the protein families according to the presence of domains and sequence relatedness to previously described proteins.

POLYX nucleic acids and polypeptides according to the invention can also be used to identify cell types based on the presence or absence of various POLYX nucleic acids according to the invention. Additional utilities for POLYX nucleic acids and polypeptides are discussed below.

POLY1 and POLY2 Nucleic Acids and Polypeptides

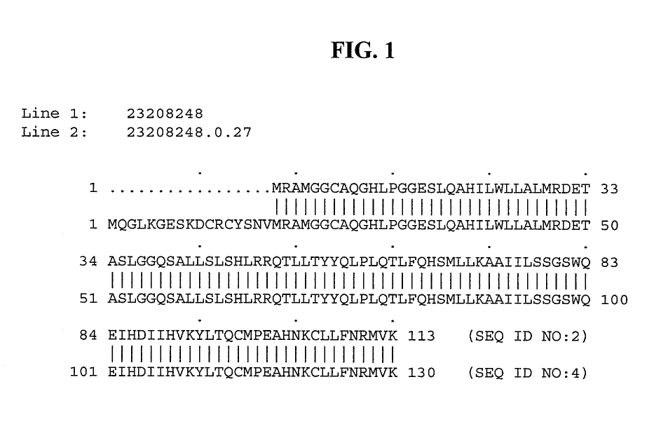

POLY1 and POLY2 nucleic acids according to the invention include the nucleic acid sequences represented in Clone 23208248 and 23208248.0.27 respectively. POLY1 and POLY2 are related by the finding that the latter clone has an extended coding sequence at the 5′, or N-terminal, end. The clones provide identical polypeptide sequences in the region of overlap (see FIG. 1). Clone 23208248 is found in the adrenal gland. A representation of the nucleotide sequence of clone 23208248 is given in Table 2. This clone includes a nucleotide sequence (SEQ ID NO:1) of 404 bp. This nucleotide sequence has an open reading frame encoding a polypeptide of 113 amino acid residues (represented in Table 1; SEQ ID NO:2) with a predicted molecular weight of 63327 Da. The start codon is at nucleotides 43-45 and the stop codon is at nucleotides 382-384. The protein of SEQ ID NO:2 is predicted by the PSORT program to localize extracellularly. The program SignalP predicts that there is no signal peptide.

TABLE 2
Translated Protein - Frame: 1 -Nucleotide 43 to 381

| | | |
|---|---|---|
| 1 | CTTAAAGGTGAGAGTAAAGACTGCAGATGCTATTCTAATGTGATG | (SEQ ID NO:1) |
| | Met | (SEQ ID NO:2) |
| 46 | AGAGCAATGGGAGGGTGTGCACAGGGTCACCTACCTGGTGGTGAG | |
| | ArgAlaMetGlyGlyCysAlaGlnGlyHisLeuProGlyGlyGlu | |
| 91 | AGCCTCCAGGCTCACATTCTATGGCTCCTGGCACTAATGAGAGAT | |
| | SerLeuGlnAlaHisIleLeuTrpLeuLeuAlaLeuMetArgAsp | |
| 136 | GAGACTGCCTCCCTAGGTGGGCAGTCAGCCCTCTTATCACTGTCT | |
| | GluThrAlaSerLeuGlyGlyGlnSerAlaLeuLeuSerLeuSer | |
| 181 | CATCTCAGAAGACAGACATTGCTGACATATTACCAGCTGCCCCTG | |
| | HisLeuArgArgGlnThrLeuLeuThrTyrTyrGlnLeuProLeu | |
| 226 | CAGACTTTATTCCAACATTCGATGCTACTAAAAGCAGCAATAATA | |
| | GlnThrLeuPheGlnHisSerMetLeuLeuLysAlaAlaIleIle | |
| 271 | CTTTCCTCAGGATCATGGCAAGAGATCCATGACATAATCCATGTA | |
| | LeuSerSerGlySerTrpGlnGluIleHisAspIleIleHisVal | |
| 316 | AAATATTTAACACAGTGCATGCCAGAAGCACATAATAAATGTTTA | |
| | LysTyrLeuThrGlnCysMetProGluAlaHisAsnLysCysLeu | |
| 361 | TTATTCAATCGCATGGTAAAATAGAAGAAATAGACAGTCCATTT | |
| | LeuPheAsnArgMetValLys | |

A search of the sequence databases using BLASTX reveals that clone 23208248 has similarity to no protein in a public or a published database.

Clone 23208248.0.27 is found in the adrenal gland. A representation of the nucleotide sequence of clone 23208248.0.27 is given in Table 3. This clone includes a nucleotide sequence (SEQ ID NO:3) of 824 bp. This nucleotide sequence has an open reading frame encoding a polypeptide of 130 amino acid residues (represented in Table 3; SEQ ID NO:4) with a predicted molecular weight of 14596 Da. The start codon is at nucleotides 371-373 and the stop codon is at nucleotides 762-764. The protein of SEQ ID NO:4 is predicted by the PSORT program to localize in the cytoplasm. The programs PSORT and SignalP predict that there may be an uncleavable amino terminal signal peptide.

TABLE 3
Translated Protein - Frame: 2 - Nucleotide 371 to 760

| | | |
|---|---|---|
| 1 | CGGTAGCAGAAAGTTGTGCTAACCATGGGAGACTGGCACGTGCTG | (SEQ ID NO:3) |
| 46 | CAGATGCTGATCTTGCTGTGCCTTTTCTCCTTTCTGTGAATAAAG | |
| 91 | CATTGTTCCAACAAGCACCTTGTATGCTGAGGTCTCCTTGGGGAT | |
| 136 | TCCAAGCCCATAGGCAATGCAGGGGCCAAGGTCAATGGGCTCCGA | |
| 181 | CTTCTCGTGATTGGTGGTGTTATACTCTTTGCCATCCTCCACAGG | |
| 226 | TTGATAATCCCCACCTGGGATTGGCAGCCTGATCTTCCTGTTTGA | |
| 271 | CACATAACATAATCTCAGGTAGGAATAACTGCTACATAGGAACAT | |
| 316 | CAAGCAGAGGAAGGAAAAAGAACATAGGGAGAATAGCAGAAGATG | |
| 361 | AAGCTGGGTCATGCAGGGCCTTAAAGGTGAGAGTAAAGACTGCAG | |
| | MetGlnGlyLeuLysGlyGluSerLysAspCysArg | (SEQ ID NO:4) |
| 406 | ATGCTATTCTAATGTGATGAGAGCAATGGGAGGGTGTGCACAGGG | |
| | CysTyrSerAsnValMetArgAlaMetGlyGlyCysAlaGlnGly | |
| 451 | TCACCTACCTGGTGGTGAGAGCCTCCAGGCTCACATTCTATGGCT | |
| | HisLeuProGlyGlyGluSerLeuGlnAlaHisIleLeuTrpLeu | |
| 496 | CCTGGCACTAATGAGAGATGAGACTGCCTCCCTAGGTGGGCAGTC | |
| | LeuAlaLeuMetArgAspGluThrAlaSerLeuGlyGlyGlnSer | |
| 541 | AGCCCTCTTATCACTGTCTCATCTCAGAAGACAGACATTGCTGAC | |
| | AlaLeuLeuSerLeuSerHisLeuArgArgGlnThrLeuLeuThr | |
| 586 | ATATTACCAGCTGCCCCTGCAGACTTTATTCCAACATTCGATGCT | |
| | TyrTyrGlnLeuProLeuGlnThrLeuPheGlnHisSerMetLeu | |
| 631 | ACTAAAAGCAGCAATAATACTTTCCTCAGGATCATGGCAAGAGAT | |
| | LeuLysAlaAlaIleIleLeuSerSerGlySerTrpGlnGluIle | |
| 676 | CCATGACATAATCCATGTAAAATATTTAACACAGTGCATGCCAGA | |
| | HisAspIleIleHisValLysTyrLeuThrGlnCysMetProGlu | |
| 721 | AGCACATAATAAATGTTTATTATTCAATCGCATGGTAAAATAGAA | |
| | AlaHisAsnLysCysLeuLeuPheAsnArgMetValLys | |
| 766 | GAATTAGACAAGTCCATTTAACACATGAAATTTATTGTAAATAAA | |
| 811 | GACAGAACCTTCAC | |

A search of the sequence databases using BLASTX reveals that clone 23208248.0.27 has similarity to no protein sequences in public or published patent databases.

An alignment of the proteins of clones 23208248 and 23208248.0.27 is shown in FIG. 1. The proteins of the invention encoded by clones 23208248 and 23208248.0.27 include the full protein disclosed as being encoded by the ORFs described herein, as well as any mature protein arising therefrom as a result of posttranslational modifications. Thus, the proteins of the invention encompass both a precursor and any active forms of the 23208248 and 23208248.0.27 proteins.

POLY3

A POLY3 nucleic acid according to the invention includes the nucleic acid sequence represented in Clone 29200321. RNA sequences homologous to this clone are found in fetal brain tissue. A representation of the nucleotide sequence of clone 29200321 is given in Table 4, which shows the negative strand from the sequence actually discovered. This clone includes a nucleotide sequence (SEQ ID NO:5) of 386 bp. The nucleotide sequence shown in Table 4 has an open reading frame encoding a polypeptide of 99 amino acid residues (represented in Table 4; SEQ ID NO:6) with a predicted molecular weight of 72993.5 Da. The start codon is at nucleotides 16-18 and the stop codon is at nucleotides 312-314. The protein of SEQ ID NO:6 is predicted by the PSORT program to localize extracellularly. The program SignalP predicts that there is a signal peptide. The protein associated with 29200321 is encoded in a negative reading frame. The sequence shown below has been reverse-complemented and renumbered to allow reading of the protein in the expected N to C direction.

TABLE 4
Translated Protein - Frame: −1 - Nucleotide 16 to 312

| | | |
|---|---|---|
| 1 | ACGCGTTCCTTTGTCATGCATAAAGACACGGCCTTAATTCTTCTG | (SEQ ID NO:5) |
| | MetHisLysAspThrAlaLeuIleLeuLeu | (SEQ ID NO:6) |
| 46 | CACAACCTGAGCTGTTTCGTTGTTTTCATTGAGGCACAGTGTCAC | |
| | HisAsnLeuSerCysPheValValPheIleGluAlaGlnCysHis | |
| 91 | CACGTAGCCCAGGCTAGCCTGAAACATGACCTTCTTCCTCATCTC | |
| | HisValAlaGlnAlaSerLeuLysHisAspLeuLeuProHisLeu | |
| 136 | TCAGATTCTAAGATTATAGGCGGGCACCATCGTGCCTGGGTGGGC | |
| | SerAspSerLysIleIleGlyGlyHisHisArgAlaTrpValGly | |
| 181 | ACTCAGCATACATTGAGGAACGCATCTCAGTGGACTGAAAGGGAT | |
| | ThrGlnHisThrLeuArgAsnAlaSerGlnTrpThrGluArgAsp | |
| 226 | GTTGAGGCTGGGCATGGTGGCAAATATGGGGACTTTCGAGGCTGC | |
| | ValGluAlaGlyHisGlyGlyLysTyrGlyAspPheArgGlyCys | |
| 271 | GGCCAGGGGAGCTTGAGCTTGGAGCCTGTCCAGACGATGCAGTGA | |
| | GlyGlnGlySerLeuSerLeuGluProValGlnThrMetGln | |
| 316 | AGTCCAGGCAATCTGGGTGACACAGTGAGGCCTTGTCTTTGAAAA | |
| 361 | TATCTCATTTGTTTGAGAGAAGATCT | |

A search of the sequence databases using BLAST P and BLASTX reveals that clone 29200321 has no significant similarity to any protein in the public or published patent databases.

The proteins of the invention encoded by clone 29200321 include the full protein disclosed as being encoded by the ORFs described herein, as well as any mature protein arising therefrom as a result of posttranslational modifications. Thus, the proteins of the invention encompass both a precursor and any active forms of the 29200321 protein.

Results presented in Example C show that clone 29200321 shows high expression relative to normal cells is found in certain ovarian cancer cell lines, and in gastric cancer and a colon cancer cell line. In addition, this clone is broadly expressed in lung cancers and certain CNS cancer cells. These results suggest that this clone may be used as a selective probe for detection or diagnosis of these cancers, and that the clones or their gene products may be useful in treatment of such cancers. In addition, this gene product has been shown in Example CC1 to inhibit serine protease activity. This property may make it useful in modulating tissue remodeling or in treating certain cancers.

POLY4

A POLY4 nucleic acid according to the invention includes the nucleic acid sequence represented in Clone 10129612_{—}1. RNA sequences homologous to this clone are found in the heart. A representation of the nucleotide sequence of clone 10129612_{—}1 is given in Table 5. This clone includes a nucleotide sequence (SEQ ID NO:7) of 1743 bp. This nucleotide sequence has an open reading frame encoding a polypeptide of 429 amino acid residues (represented in Table D; SEQ ID NO:8) with a predicted molecular weight of 47068.8 Da. The start codon is at nucleotides 437-439 and the stop codon is at nucleotides 1724-1726. The protein of SEQ ID NO:8 is predicted by the PSORT program to localize in the endoplasmic reticulum (membrane) with a certainty of 0.8500. The programs PSORT and SignalP predict that there is no signal peptide.

TABLE 5
Translated Protein - Frame: 2 - Nucleotide 437 to 1723

| | | |
|---|---|---|
| 1 | TGCTTCTCTCCCTCTCTCCTCGCTGTCTTTCCCTCGGTCATTGTT | (SEQ ID NO:7) |
| 46 | CTCTCTCTCCTCCTGCCTTTGATGCACATACGTTGTCACAATTCA | |
| 91 | TTGACTCTCCTCTCTTCTCTGTTCTTACACTCAAGCCTAACGGTG | |
| 136 | CTTTCGCCAGGCAGCAGCAGCCTCTCCTGCGAGTTTTAGGCATGT | |
| 181 | ATGCAGCTCAGTTTGATCGAGCGTTCCTTTTCTGCCTTTTCACTC | |
| 226 | TTACAAAAGATTAAAAGGTGGCGTCACATTGCTCCCCTGTTCCTT | |
| 271 | CCCGCAGGAGGGACTTAAAAGGGACAACAAAAACTAATCACTTTC | |
| 316 | AATAAGCATTTTCTTGCTGGAAAAAAAAAAGAAAAGAAAAAAAAA | |
| 361 | GAAAGAAAAAAAAAGGCGGGGGGTGGACTTAGCAGTGTAATTTGA | |
| 406 | GACCGGTGGTAAGGATTGGAGCGAGCTAGAGATGCTGCACGCTGC | |
| | MetLeuHisAlaAl | (SEQ ID NO:8) |
| 451 | TAACAAGGGAAGGAAGCCTTCAGCTGAGGCAGGTCGTCCCATTCC | |
| | aAsnLysGlyArgLysProSerAlaGluAlaGlyArgProIlePr | |
| 496 | ACCTACATCCTCGCCTAGTCTCCTCCCATCTGCTCAGCTGCCTAG | |
| | oProThrSerSerProSerLeuLeuProSerAlaGlnLeuProSe | |
| 541 | CTCCCATAATCCTCCACCAGTTAGCTGCCAGATGCCATTGCTAGA | |
| | rSerHisAsnProProProValSerCysGlnMetProLeuLeuAs | |
| 586 | CAGCAACACCTCCCATCAAATCATGGACACCAACCCTGATGAGGA | |
| | pSerAsnThrSerHisGlnIleMetAspThrAsnProAspGluGl | |
| 631 | ATTCTCCCCCAATTCATACCTGCTCAGAGCATGCTCAGGGCCCCA | |
| | uPheSerProAsnSerTyrLeuLeuArgAlaCysSerGlyProGl | |
| 676 | GCAAGCCTCCAGCAGTGGCCCTCCGAACCACCACAGCCAGTCGAC | |
| | nGlnAlaSerSerSerGlyProProAsnHisHisSerGlnSerTh | |
| 721 | TCTGAGGCCCCCTCTCCCACCCCCTCACAACCACACGCTGTCCCA | |
| | rLeuArgProProLeuProProProHisAsnHisThrLeuSerHi | |
| 766 | TCACCACTCGTCCGCCAACTCCCTCAACAGGAACTCACTGACCAA | |
| | sHisHisSerSerAlaAsnSerLeuAsnArgAsnSerLeuThrAs | |
| 811 | TCGGCGGAGTCAGATCCACGCCCCGGCCCCAGCGCCCAATGACCT | |
| | nArgArgSerGlnIleHisAlaProAlaProAlaProAsnAspLe | |
| 856 | GGCCACCACACCAGAGTCCGTTCAGCTTCAGGACAGCTGGGTGCT | |
| | uAlaThrThrProGluSerValGlnLeuGlnAspSerTrpValLe | |
| 901 | AAACAGCAACGTGCCACTGGAGACCCGGCACTTCCTCTTCAAGAC | |
| | uAsnSerAsnValProLeuGluThrArgHisPheLeuPheLysTh | |
| 946 | CTCCTCGGGGAGCACACCCTTGTTCAGCAGCTCTTCCCCGGGATA | |
| | rSerSerGlySerThrProLeuPheSerSerSerSerProGlyTy | |
| 991 | CCCTTTGACCTCAGGAACGGTTTACACGCCCCCGCCCCGCCTGCT | |
| | rProLeuThrSerGlyThrValTyrThrProProProArgLeuLe | |
| 1036 | GCCCAGGAATACTTTCTCCAGGAAGGCTTTCAAGCTGAAGAAGCC | |
| | uProArgAsnThrPheSerArgLysAlaPheLysLeuLysLysPr | |
| 1081 | CTCCAAATACTGCAGCTGGAAATGTGCTGCCCTCTCCGCCATTGC | |
| | oSerLysTyrCysSerTrpLysCysAlaAlaLeuSerAlaIleAl | |
| 1126 | CGCGGCCCTCCTCTTGGCTATTTTGCTGGCGTATTTCATAGTGCC | |
| | aAlaAlaLeuLeuLeuAlaIleLeuLeuAlaTyrPheIleValPr | |
| 1171 | CTGGTCGTTGAAAAACAGCAGCATAGACAGTGGTGAAGCAGAAGT | |
| | oTrpSerLeuLysAsnSerSerIleAspSerGlyGluAlaGluVa | |
| 1216 | TGGTCGGCGGGTAACACAAGAAGTCCCACCAGGGGTGTTTTGGAG | |
| | lGlyArgArgValThrGlnGluValProProGlyValPheTrpAr | |
| 1261 | GTCACAAATTCACATCAGTCAGCCCCAGTTCTTAAAGTTCAACAT | |
| | gSerGlnIleHisIleSerGlnProGlnPheLeuLysPheAsnIl | |
| 1306 | CTCCCTCGGGAAGGACGCTCTCTTTGGTGTTTACATAAGAAGAGG | |
| | eSerLeuGlyLysAspAlaLeuPheGlyValTyrIleArgArgGl | |
| 1351 | ACTTCCACCATCTCATGCCCAGTATGACTTCATGGAACGTCTGGA | |
| | yLeuProProSerHisAlaGlnTyrAspPheMetGluArgLeuAs | |
| 1396 | CGGGAAGGAGAAGTGGAGTGTGGTTGAGTCTCCCAGGGAACGCCG | |
| | pGlyLysGluLysTrpSerValValGluSerProArgGluArgAr | |
| 1441 | GAGCATACAGACCTTGGTTCAGAATGAAGCCGTGTTTGTGCAGTA | |
| | gSerIleGlnThrLeuValGlnAsnGluAlaValPheValGlnTy | |
| 1486 | CCTGGATGTGGGCCTGTGGCATCTGGCCTTCTACAATGATGGAAA | |
| | rLeuAspValGlyLeuTrpHisLeuAlaPheTyrAsnAspGlyLy | |
| 1531 | AGACAAAGAGATGGTTTCCTTCAATACTGTTGTCCTAGATGATTC | |
| | sAspLysGluMetValSerPheAsnThrValValLeuAspAspSe | |
| 1576 | AGTGCAGGACTGTCCACGTAACTGCCATGGGAATGGTGAATGTGT | |
| | rValGlnAspCysProArgAsnCysHisGlyAsnGlyGluCysVa | |
| 1621 | GTCCGGGGTGTGTCACTGTTTCCCAGGATTTCTAGGAGCAGACTG | |
| | lSerGlyValCysHisCysPheProGlyPheLeuGlyAlaAspCy | |
| 1666 | TGCTAAAGACCTTCCTGCCTTGACTTTCTGCAAGACAATCATTAA | |
| | sAlaLysAspLeuProAlaLeuThrPheCysLysThrIleIleAs | |
| 1711 | TAAAGCTGCTCTGTAAATACTAAAAAAAAAACA | |
| | nLysAlaAlaLeu | |

A search of the sequence databases using BLAST P and BLASTX reveals that clone 10129612_{—}1 has 167 of 169 residues (98%) identical to, and 168 of 169 residues (99%) positive with the 2764 residue mouse TEN-M2 protein (ACC:BAA77397). It was also found to have 74 of 134 residues (55%) identical to, and 96 of 134 residues (71%) positive with the 768 residue human protein gamma-heregulin (ACC:O14667).

The proteins of the invention encoded by clone 10129612_{—}1 include the full protein disclosed as being encoded by the ORFs described herein, as well as any mature protein arising therefrom as a result of posttranslational modifications. Thus, the proteins of the invention encompass both a precursor and any active forms of the 10129612_{—}1 protein.

Results presented in Example C suggest that clone 10129612_{—}1 may be used as a selective probe for detection or diagnosis of these cancers, and that the clones or their gene products may be useful in treatment of such cancers.

Heregulin is also known as neu differentiation factor (NDF) or glial growth factor 2 (GGF2). Heregulin is the ligand for HER-2/ErbB2/NEU, a proto-oncogene receptor tyrosine kinase implicated in breast and prostate cancer progression that was originally identified in rat neuro/glioblastoma cell lines. Ectopic expression of HER-2/ErbB2/NEU in MDA-MB-435 breast adenocarcinoma cells confers chemoresistance to taxol-induced apoptosis relative to vector transfected control cells (Yu et al. Overexpression of ErbB2 blocks taxol-induced apoptosis by up-regulation of p21Cip1, which inhibits p34Cdc2 kinase. Molec. Cell 2: 581-591, 1998). 101129612.0.19 is also related to neurestin (Otaki J M, Firestein S Dev Biol Aug. 1, 1999, 212(1):165-81). Neurestin is a putative transmembrane molecule implicated in neuronal development.

Neurestin shows homology to a neuregulin gene product, human gamma-heregulin, a Drosophila receptor-type pair-rule gene product, Odd Oz (Odz)/Ten(m), and Ten(a). Putative roles in synapse formation and morphogenesis. A mouse neurestin homolog, DOC4, has independently been isolated from the NIH-3T3. DOC4 is also known as tenascin M (TNM), Drosophila pair-rule gene homolog containing extracellular EGF-like repeats.

The tenascins constitute a family of extracellular matrix proteins that play prominent roles in tissue interactions critical to embryogenesis. Overexpression of tenascins has been described in multiple human solid malignancies. The role of the tenascin family of related proteins is to regulate epithelial-stromal interactions and participate in fibronectin-dependent cell attachment and interaction. Indeed, tenascin-C (TN) is overexpressed in the stroma of malignant ovarian tumours particularly at the interface between epithelia and stroma leading to suggestions that it may be involved in the process of invasion (Wilson et al (1996) Br J Cancer 74: 999-1004). Tenascin-C is considered a therapeutic target for certain malignant brain tumors (Gladson C L: J Neuropathol Exp Neurol October 1999;58(10): 1029-40).

Stromal or moderate to strong periductal Tn-C expression in DCIS correlates with tumor cell invasion. (Jahkola et al. Eur J Cancer October 1998;34(11):1687-92 Expression of tenascin-C in intraductal carcinoma of human breast: relationship to invasion; Jahkola T, et al. Tenascin-C expression in invasion border of early breast cancer: a predictor of local and distant recurrence. Br J Cancer. December 1998;78(11):1507-13).

Tenascin (TN) is an extracellular matrix protein found in areas of cell migration during development and expressed at high levels in migratory glioma cells (Treasurywala S, Berens M E Glia October 1998;24(2):236-43 Migration arrest in glioma cells is dependent on the alpha V integrin subunit. Phillips G R, Krushel L A, Crossin K L J Cell Sci April 1998; 111 (Pt 8):1095-104 Domains of tenascin involved in glioma migration).

Tenascin expression in hormone-dependent tissues of breast and endometrium indicate that Tenascin expression reflects malignant progression (Vollmer et al. Cancer Res Sep. 1, 1992;52(17):4642-8 Down-regulation of tenascin expression by antiprogestins during terminal differentiation of rat mammary tumors).

Potential Role of 10129612-1 in Oncologic Disease Progression

Based on the bioactivity described in the medical literature for related molecules, 10129612-1 may play a role in one or more aspects of tumor cell biology that alter the interactions of tumor epithelial cells with stromal components. In consideration, 10129612-1 may play a role in the following malignant properties: autocrine/paracrine stimulation of tumor cell proliferation; autocrine/paracrine stimulation of tumor cell survival and tumor cell resistance to cytotoxic therapy; local tissue remodeling, paranechmal and basement membrane invasion and motility of tumor cells thereby contributing to metastasis; and tumor-mediated immunosuppression of T-cell mediated immune effector cells and pathways resulting in tumor escape from immune surveilance.

Therapeutic Interventions Targeting 10129612-1 in Oncologic Indications

Predicted disease indications from expression profiling (see Example 5) include a subset of human gliomas, astrocytomas, mixed glioma/astrocytomas, renal cells carcinoma, breast adenocarcinoma, ovarian cancer, melanomas. Targeting of 10129612-1 by human or humanized monoclonal antibodies designed to disrupt predicted interactions of 10129612-1 with its cognate receptor may result in significant anti-tumor/anti-metastatic activity and the amelioration of associated symptomatology. Identification of small molecules that specifically/selectively interfere with downstream signaling components engaged by 10129612-1/receptor interactions would also be expected to result in significant anti-tumor/anti-metastatic activity and the amelioration of associated symptomatology. Likewise, modified antisense ribonucleotides or antisense gene expression constructs (plasmids, adenovirus, adeno-associated viruses, “naked” DNA approaches) designed to diminish the expression of 10129612-1 transcripts/messenger RNA (mRNA) would be anticipated based on predicted properties of 10129612-1 to have anti-tumor impact.

The neuregulin, glial growth factor 2, diminishes autoimmune demyelination and enhances remyelination in a chronic relapsing model for multiple sclerosis. (Cannella et al. . Proc. Nat. Acad. Sci. 95: 10100-10105, 1998; Notterpek L M and Rome L H, Dev Neurosci 1994;16(5-6):267-78).

Otaki and Firestein (Dev Biol Aug. 1, 1999;212(1):165-81) reported that, as detected by Northern blot analysis, neurestin is highly expressed in the brain and relatively low in other tissues. In situ hybridization to tissue sections demonstrates that neurestin is expressed in many types of neurons, including pyramidal cells in the cerebral cortex and tufted cells in the olfactory bulb during development. In adults, neurestin is mainly expressed in olfactory and hippocampal granule cells. Nonetheless, in adults neurestin expression can be induced in external tufted cells during regeneration of olfactory sensory neurons.

Direct delivery of recombinant purified 10129612-1 or fragments of 10129612-1 into brain parenchymal regions may promote the regeneration/repair/remylination of injured central nervous system cells resulting from ischemia, brain trauma and various neurodegenerative diseases.

POLY5

A POLY5 nucleic acid according to the invention includes the nucleic acid sequence represented in Clone10168180.0.35. RNA sequences homologous to this clone are found in the spleen. A representation of the nucleotide sequence of clone 10168180.0.35 is given in Table 6. This clone includes a nucleotide sequence (SEQ ID NO:9) of 2450 bp. This nucleotide sequence has an open reading frame encoding a polypeptide of 386 amino acid residues (represented in Table 6; SEQ ID NO:10) with a predicted molecular weight of 45140.7 Da. The start codon is at nucleotides 380-382 and the stop codon is at nucleotides 1538-1540. The protein of SEQ ID NO:10 is predicted by the PSORT program to localize to the mitochondrial inner membrane with a certainty of 0.8219. The programs PSORT and SignalP predict that there is no signal peptide.

TABLE 6
Translated Protein - Frame: 2 - Nucleotide 380 to 1537

| | | |
|---|---|---|
| 1 | ATTTTTGAAAGCAAAATAAGGTTTTCTTTTTTCCCCTTTCTTGTA | (SEQ ID NO:9) |
| 46 | ATAAATGATAAAATTCCTGTGCAGGTGGCAGATTTTTAAACTATA | |
| 91 | CTGAGTCAACGGTGCAGGTAAACCACCCTCGGGCCCAGTCCTAGA | |
| 136 | GTAGACACAAGATCGCCTGGGAGGGCCGCTGGCCCCTCTAACGCT | |
| 181 | CTGGCTGTCAGACTTGGGACAAGTCCCTTAACCATTAAATATATT | |
| 226 | TGGTCCCCAAATTTTGAATGGCAATAAACTGGAGACACTTTCTTT | |
| 271 | AGTAAGTTGCTTTGCTAACAACACACCCTTGGAACACTTGGATCT | |
| 316 | GAGTCAAAATCTATTACAACATAAAAATGATGAAAATTGCTCATG | |
| 361 | GCCAGAAACTGTGGTCAATATGAATCTGTCATACAATAAATTGTC | |
| | MetAsnLeuSerTyrAsnLysLeuSe | (SEQ ID NO:10) |
| 406 | TGATTCTGTCTTCAGGTGCTTGCCCAAAAGTATTCAAATACTTGA | |
| | rAspSerValPheArgCysLeuProLysSerIleGlnIleLeuAs | |
| 451 | CCTAAATAATAACCAAATCCAAACTGTACCTAAAGAGACTATTCA | |
| | pLeuAsnAsnAsnGlnIleGlnThrValProLysGluThrIleHi | |
| 496 | TCTGATGGCCTTACGAGAACTAAATATTGCATTTAATTTTCTAAC | |
| | sLeuMetAlaLeuArgGluLeuAsnIleAlaPheAsnPheLeuTh | |
| 541 | TGATCTCCCTGGATGCAGTCATTTCAGTAGACTTTCAGTTCTGAA | |
| | rAspLeuProGlyCysSerHisPheSerArgLeuSerValLeuAs | |
| 586 | CATTGAAATGAACTTCATTCTCAGCCCATCTCTGGATTTTGTTCA | |
| | nIleGluMetAsnPheIleLeuSerProSerLeuAspPheValGl | |
| 631 | GAGCTGCCAGGAAGTTAAAACTCTAAATGCGGGAAGAAATCCATT | |
| | nSerCysGlnGluValLysThrLeuAsnAlaGlyArgAsnProPh | |
| 676 | CCGGTGTACCTGTGAATTAAAAAATTTCATTCAGCTTGAAACATA | |
| | eArgCysThrCysGluLeuLysAsnPheIleGlnLeuGluThrTy | |
| 721 | TTCAGAGGTCATGATGGTTGGATGGTCAGATTCATACACCTGTGA | |
| | rSerGluValMetMetValGlyTrpSerAspSerTyrThrCysGl | |
| 766 | ATACCCTTTAAACCTAAGGGGAACTAGGTTAAAAGACGTTCATCT | |
| | uTyrProLeuAsnLeuArgGlyThrArgLeuLysAspValHisLe | |
| 811 | CCACGAATTATCTTGCAACACAGCTCTGTTGATTGTCACCATTGT | |
| | uHisGluLeuSerCysAsnThrAlaLeuLeuIleValThrIleVa | |
| 856 | GGTTATTATGCTAGTTCTGGGGTTGGCTGTGGCCTTCTGCTGTCT | |
| | lValIleMetLeuValLeuGlyLeuAlaValAlaPheCysCysLe | |
| 901 | CCACTTTGATCTGCCCTGGTATCTCAGGATGCTAGGTCAATGCAC | |
| | uHisPheAspLeuProTrpTyrLeuArgMetLeuGlyGlnCysTh | |
| 946 | ACAAACATGGCACAGGGTTAGGAAAACAACCCAAGAACAACTCAA | |
| | rGlnThrTrpHisArgValArgLysThrThrGlnGluGlnLeuLy | |
| 991 | GAGAAATGTCCGATTCCACGCATTTATTTCATACAGTGAACATGA | |
| | sArgAsnValArgPheHisAlaPheIleSerTyrSerGluHisAs | |
| 1036 | TTCTCTGTGGGTGAAGAATGAATCGATCCCCAATCTAGAGAAGGA | |
| | pSerLeuTrpValLysAsnGluSerIleProAsnLeuGluLysGl | |
| 1081 | AGATGGTTCTATCTTGATTTGCCTTTATGAAAGCTACTTTGACCC | |
| | uAspGlySerIleLeuIleCysLeuTyrGluSerTyrPheAspPr | |
| 1126 | TGGCAAAAGCATTAGTGAAAATATTGTAAGCTTCATTGAGAAAAG | |
| | oGlyLysSerIleSerGluAsnIleValSerPheIleGluLysSe | |
| 1171 | CTATAAGTCCATCTTTGTTTTGTCTCCCAACTTTGTCCAGAATGA | |
| | rTyrLysSerIlePheValLeuSerProAsnPheValGlnAsnGl | |
| 1216 | GTGGTGCCATTATGAATTCTACTTTGCCCACCACAATCTCTTCCA | |
| | uTrpCysHisTyrGluPheTyrPheAlaHisHisAsnLeuPheHi | |
| 1261 | TGAAAATTCTGATCATATAATTCTTATCTTACTGGAACCCATTCC | |
| | sGluAsnSerAspHisIleIleLeuIleLeuLeuGluProIlePr | |
| 1306 | ATTCTATTGCATTCCCACCAGGTATCATAAACTGAAAGCTCTCCT | |
| | oPheTyrCysIleProThrArgTyrHisLysLeuLysAlaLeuLe | |
| 1351 | GGAAAAAAAAGCATACTTGGAATGGCCCAAGGATAGGCGTAAATG | |
| | uGluLysLysAlaTyrLeuGluTrpProLysAspArgArgLysCy | |
| 1396 | TGGGCTTTTCTGGGCAAACCTTCGAGCTGCTATTAATGTTAATGT | |
| | sGlyLeuPheTrpAlaAsnLeuArgAlaAlaIleAsnValAsnVa | |
| 1441 | ATTAGCCACCAGAGAAATGTATGAACTGCAGACATTCACAGAGTT | |
| | lLeuAlaThrArgGluMetTyrGluLeuGlnThrPheThrGluLe | |
| 1486 | AAATGAAGAGTCTCGAGGTTCTACAATCTCTCTGATGAGAACAGA | |
| | uAsnGluGluSerArgGlySerThrIleSerLeuMetArgThrAs | |
| 1531 | TTGTCTATAAAATCCCACAGTCCTTGGGAAGTTGGGGACCACATA | |
| | pCysLeu | |
| 1576 | CACTGTTGGGATGTACATTGATACAACCTTTATGATGGCAATTTG | |
| 1621 | ACAATATTTATTAAAATAAAAAATGGTTATTCCCTTCATATCAGT | |
| 1666 | TTCTAGAAGGATTTCTAAGAATGTATCCTATAGAAACACCTTCAC | |
| 1711 | AAGTTTATAAGGGCTTATGGAAAAAGGTGTTCATCCCAGGATTGT | |
| 1756 | TTATAATCATGAAAAATGTGGCCAGGTGCAGTGGCTCACTCTTGT | |
| 1801 | AATCCCAGCACTATGGGAGGCCAAGGTGGGTGAACCACGAGGTCA | |
| 1846 | AGAGATGGAGACCATCCTGGCCAACATGGTGAAACCCTGTCTCTA | |
| 1891 | CTAAAAATACAAAAATTAGCTGGGCGTGATGGTGCATGCCTGTAG | |
| 1936 | TCCCAGCTACTTGGGAGGCTGAGGCAGGAGAATCGCTTGAACCCG | |
| 1981 | GGAGGTGGCAGTTGCAGTGAGCTGAGATCGAGCCACTGCACTCCA | |
| 2026 | GCCTGGTGACAGAGCGAGACTCCATCTCCAAAAAAAAGAAAAAAA | |
| 2071 | AAAAGAAAAAAATGGAAAACATCCTCATGGCCACAAAATAAGGTC | |
| 2116 | TAATTCAATAAATTATAGTACATTAATGTAATATAATATTACATG | |
| 2161 | CCACTAAAAAGAATAAGGTAGCTGTATATTTCCTGGTATGGAAAA | |
| 2206 | AACATATTAATATGTTATAAACTATTAGGTTGGTGCAAAACTAAT | |
| 2251 | TGTGGTTTTTGCCATTGAAATGGCATTGAAATAAAAGTGTAAAGA | |
| 2296 | AATCTATACCAGATGTAGTAACAGTGGTTTGGGTCTGGGAGGTTG | |
| 2341 | GATTACAGGGAGCATTTGATTTCTATGTTGTGTATTTCTATAATG | |
| 2386 | TTTGAATTGTTTAGAATGAATCTGTATTTCTTTTATAAGTAGAAA | |
| 2431 | AAAAAAAAAAAAAAAAAAAA | |

A search of the sequence databases using BLAST P and BLASTX reveals that the protein of clone 10168180.0.35 has 219 of 354 residues (61%) identical to, and 278 of 354 residues (78%) positive with the 786 residue human TOLL-like receptor 1 (ACC:O15452). In addition, the protein has 433 of 434 residues (99%) identical to and positive with the 811 residue human Toll protein PRO358 (PCT publication WO9920756-A2 published Apr. 29, 1999).

The proteins of the invention encoded by clone10168180.0.35 include the full protein disclosed as being encoded by the ORF described herein, as well as any mature protein arising therefrom as a result of posttranslational modifications. Thus, the proteins of the invention encompass both a precursor and any active forms of the 10168180.0.35 protein.

POLY6

A POLY6 nucleic acid according to the invention includes the nucleic acid sequence represented in Clone 10354784.0.148 (also referred to as CG50670-02). RNA sequences homologous to this clone are found in the pituitary gland, as well as in kidney, heart, and thalamus. A representation of the nucleotide sequence of clone 10354784.0.148 is given in Table 7. This clone includes a nucleotide sequence (SEQ ID NO:11) of 3550 bp. This nucleotide sequence has an open reading frame encoding a polypeptide of 735 amino acid residues (represented in Table 7; SEQ ID NO:12) with a predicted molecular weight of 79802 Da. The start codon is at nucleotides 728-730 and the stop codon is at nucleotides 2933-2935. The protein of SEQ ID NO:12 is predicted by the PSORT program to localize in the plasma membrane with a certainty of 0.4600. The programs PSORT and SignalP predict that there is a signal peptide, with the most likely cleavage site between residues 25 and 26: STA-EA.

TABLE 7
Translated Protein - Frame: 2 - Nucleotide 728 to 2932

| | | |
|---|---|---|
| 1 | GTATCATTTTCCATTCTTTTTGGGGCCTCCGAAACTGTATAAATT | (SEQ ID NO:11) |
| 46 | TCAGGTTTTAGAAAACCTGGGTGTGTCCCTGGTTGGCATATAAAG | |
| 91 | CGGAATCACACATAGTCCCCTTGCTCCTTGAAGGTTGCTGAGGAA | |
| 136 | CGGCACACATTAGAGAGTAAACAGGCCTTTCAGTGAGTTCTCTGC | |
| 181 | AGTTTGTCCACAGTGTTGAAAAAAGATTACAGCTTTCCCAGCTGT | |
| 226 | GCACCTGAGGAAGTACATAGGTGATTTGCATTTGGGGACCTTGCA | |
| 271 | ATATGAGAAATGCATGTGTTTAAACAGTGGATTCCATTCAGCTCA | |
| 316 | GCCGGAGGCCGGCTCTGAGATGCTCACTGAGAGACAGTTGGGCCT | |
| 361 | GAGAACCATAGGGTGGGGTTGAGAGCATGGCAGATTCTTGTTTCC | |
| 406 | CATCTCATCTTCAGCCTCACAGCGCACATACTGAGTGCAAGCAGA | |
| 451 | AAGAAATATCTGTACCATTTAAACTGCCTCTACACTCCCTCACCT | |
| 496 | TTCTCTCTTTGCCAGCACACAGTTAACTGTGCATATGTTATGTTG | |
| 541 | ATGCTGCTGTTCTTCTGTGTTATCTCATTTCTTACTCATAACAGC | |
| 586 | TCCCTGCAGAAGCAGTCCTTGTTTCTGATAAGGACACCAAGCCCC | |
| 631 | AAGGGAATTCTGTAGCACGCCCCACTCTACATAGGTTGAAAGACC | |
| 676 | CGGAATGGCTGTTTGATCCCATCTCCATGCTCTCTGGGACTGCCT | |
| 721 | CCTGGGCATGCTCTACNAGGACATCCTGGTNNNCCACACGCCTTC | |
| | MetLeuTyr---AspIleLeuVal---HisThrproSe | (SEQ ID NO:12) |
| 766 | TGTCCTTGCCCTCCTTGCCCCTCCAGGCTCCACCGCTGAGGCTGC | |
| | rValLeuAlaLeuLeuAlaProProGlySerThrAlaGluAlaAl | |
| 811 | CCGCATCATCTACCCCCCAGAGGCCCAAACCATCATTGTCACCAA | |
| | aArgIleIleTyrProProGluAlaGlnThrIleIleValThrLy | |
| 856 | AGGCCAGAGTCTCATTCTGGAGTGTGTGGCCAGTGGAATCCCACC | |
| | sGlyGlnSerLeuIleLeuGluCysValAlaSerGlyIleProPr | |
| 901 | CCCACGGGTCACCTGGGCCAAGGATGGGTCCAGTGTCACCGGCTA | |
| | oProArgValThrTrpAlaLysAspGlySerSerValThrGlyTy | |
| 946 | CAACAAGACGCGCTTCCTGCTGAGCAACCTCCTCATCGACACCAC | |
| | rAsnLysThrArgPheLeuLeuSerAsnLeuLeuIleAspThrTh | |
| 991 | CAGCGAGGAGGACTCAGGCACCTCCCGGTGCATGCCCGACAATGG | |
| | rSerGluGluAspSerGlyThrSerArgCysMetProAspAsnGl | |
| 1036 | GGTTGGGCAGCCCGGGGCAGCGGTCATCCTCTACAATGTCCAGGT | |
| | yValGlyGlnProGlyAlaAlaValIleLeuTyrAsnValGlnVa | |
| 1081 | GTTTGAACCCCCTGAGGTCACCATGGAGCTATCCCAGCTGGTCAT | |
| | lPheGluProProGluValThrMetGluLeuSerGlnLeuValIl | |
| 1126 | CCCCTGGGGCCAGAGTGCCAAGCTTACCTGTGAGGTGCGTGGGAA | |
| | eProTrpGlyGlnSerAlaLysLeuThrCysGluValArgGlyAs | |
| 1171 | CCCCCCGCCCTCCGTGCTGTGGCTGAGGAATGCTGTGCCCCTCAT | |
| | nProProProSerValLeuTrpLeuArgAsnAlaValProLeuIl | |
| 1216 | CTCCAGCCAGCGCCTCCGGCTCTCCCGCAGGGCCCTGCGCGTGCT | |
| | eSerSerGlnArgLeuArgLeuSerArgArgAlaLeuArgValLe | |
| 1261 | CAGCATGGGGCCTGAGGACGAAGGCGTCTACCAGTGCATGGCCGA | |
| | uSerMetGlyProGluAspGluGlyValTyrGlnCysMetAlaGl | |
| 1306 | GAACGAGGTTGGGAGCGCCCATGCCGTAGTCCAGCTGCGGACCTC | |
| | uAsnGluValGlySerAlaHisAlaValValGlnLeuArgThrSe | |
| 1351 | CAGGCCAAGCATAACCCCAAGGCTATGGCAGGATGCTGAGCTGGC | |
| | rArgProSerIleThrProArgLeuTrpGlnAspAlaGluLeuAl | |
| 1396 | TACTGGCACACCTCCTGTATCACCCTCCAAACTCGGCAACCCTGA | |
| | aThrGlyThrProProValSerProSerLysLeuGlyAsnProGl | |
| 1441 | GCAGATGCTGAGGGGGCAACCGGCGCTCCCCAGACCCCCAACGTC | |
| | uGlnMetLeuArgGlyGlnProAlaLeuProArgProProThrSe | |
| 1486 | AGTGGGGCCTGCTTCCCCGCAGTGTCCAGGAGAGAAGGGGCAGGG | |
| | rValGlyProAlaSerProGlnCysProGlyGluLysGlyGlnGl | |
| 1531 | GGCTCCCGCCGAGGCTCCCATCATCCTCAGCTCGCCCCGCACCTC | |
| | yAlaProAlaGluAlaProIleIleLeuSerSerProArgThrSe | |
| 1576 | CAAGACAGACTCATATGAACTGGTGTGGCGGCCTCGGCATGAGGG | |
| | rLysThrAspSerTyrGluLeuValTrpArgProArgHisGluGl | |
| 1621 | CAGTGGCCGGGCGCCAATCCTCTACTATGTGGTGAAACACCGCAA | |
| | ySerGlyArgAlaProIleLeuTyrTyrValValLysHisArgLy | |
| 1666 | GCAGGTCACAAATTCCTCTGACGATTGGACCATCTCTGGCATTCC | |
| | sGlnValThrAsnSerSerAspAspTrpThrIleSerGlyIlePr | |
| 1711 | AGCCAACCAGCACCGCCTGACCCTCACCAGACTTGACCCCGGGAG | |
| | oAlaAsnGlnHisArgLeuThrLeuThrArgLeuAspProGlySe | |
| 1756 | CTTGTATGAAGTGGAGATGGCAGCTTACAACTGTGCGGGAGAGGG | |
| | rLeuTyrGluValGluMetAlaAlaTyrAsnCysAlaGlyGluGl | |
| 1801 | CCAGACAGCCATGGTCACCTTCCGAACTGGACGGCGGCCCAAACC | |
| | yGlnThrAlaMetValThrPheArgThrGlyArgArgProLysPr | |
| 1846 | CGAGATCATGGCCAGCAAGGAGCAGCAGATCCAGAGAGACGACCC | |
| | oGluIleMetAlaSerLysGluGlnGlnIleGlnArgAspAspPr | |
| 1891 | TGGAGCCAGTCCCCAGAGCAGCAGCCAGCCAGACCACGGCCGCCT | |
| | oGlyAlaSerProGlnSerSerSerGlnProAspHisGlyArgLe | |
| 1936 | CTCCCCCCCAGAAGCTCCCGACAGGCCCACCATCTCCACGGCCTC | |
| | uSerProProGluAlaProAspArgProThrIleSerThrAlaSe | |
| 1981 | CGAGACCTCAGTGTACGTGACCTGGATTCCCCGTGGGAATGGTGG | |
| | rGluThrSerValTyrValThrTrpIleProArgGlyAsnGlyGl | |
| 2026 | GTTCCCAATCCAGTCCTTCCGTGTGGAGTACAAGAAGCTAAAGAA | |
| | yPheProIleGlnSerPheArgValGluTyrLysLysLeuLysLy | |
| 2071 | AGTGGGAGACTGGATTCTGGCCACCAGCGCCATCCCCCCATCGCG | |
| | sValGlyAspTrpIleLeuAlaThrSerAlaIleProProSerAr | |
| 2116 | GCTGTCCGTGGAGATCACGGGCCTAGAGAAAGGAGCCTCCTACAA | |
| | gLeuSerValGluIleThrGlyLeuGluLysGlyAlaSerTyrLy | |
| 2161 | GTTTCGAGTCCGGGCTCTGAACATGCTGGGGGAGAGCGAGCCCAG | |
| | sPheArgValArgAlaLeuAsnMetLeuGlyGluSerGluProSe | |
| 2206 | CGCCCCCTCTCGGCCCTACGTGGTGTCGGGCTACAGCGGTCGCGT | |
| | rAlaProSerArgProTyrValValSerGlyTyrSerGlyArgVa | |
| 2251 | GTACGAGAGGCCCGTGGCAGGTCCTTATATCACCTTCACGGATGC | |
| | lTyrGluArgProValAlaGlyProTyrIleThrPheThrAspAl | |
| 2296 | GGTCAATGAGACCACCATCATGCTCAAGTGGATGTACATCCCAGC | |
| | aValAsnGluThrThrIleMetLeuLysTrpMetTyrIleProAl | |
| 2341 | AAGTAACAACAACACCCCAATCCATGGCTTTTATATCTATTATCG | |
| | aSerAsnAsnAsnThrProIleHisGlyPheTyrIleTyrTyrAr | |
| 2386 | ACCCACAGACAGTGACAATGATAGTGACTACAAGAAGGATATGGT | |
| | gProThrAspSerAspAsnAspSerAspTyrLysLysAspMetVa | |
| 2431 | GGAAGGGGACAAGTACTGGCACTCCATCAGCCACCTGCAGCCAGA | |
| | lGluGlyAspLysTyrTrpHisSerIleSerHisLeuGlnProGL | |
| 2476 | GACCTCCTACGACATTAAGATGCAGTGCTTCAATGAAGGAGGGGA | |
| | uThrSerTyrAspIleLysMetGlnCysPheAsnGluGlyGlyGl | |
| 2521 | GAGCGAGTTCAGCAACGTGATGATCTGTGAGACCAAAGCTCGGAA | |
| | uSerGluPheSerAsnValMetIleCysGluThrLysAlaArgLy | |
| 2566 | GTCTTCTGGCCAGCCTGGTCGACTGCCACCCCCAACTCTGGCCCC | |
| | sSerSerGlyGlnProGlyArgLeuProProProThrLeuAlaPr | |
| 2611 | ACCACAGCCGCCCCTTCCTGAAACCATAGAGCGGCCGGTGGGCAC | |
| | oProGlnProProLeuProGluThrIleGluArgProValGlyTh | |
| 2656 | TGGGGCCATGGTGGCTCGCTCCAGCGACGTGCCCTATCTGATTGT | |
| | rGlyAlaMetValAlaArgSerSerAspValProTyrLeuIleVa | |
| 2701 | CGGGGTCGTCCTGGGCTCCATCGTTCTCATCATCGTCACCTTCAT | |
| | lGlyValValLeuGlySerIleValLeuIleIleValThrPheIl | |
| 2746 | CCCCTTCTGCTTGTGGAGGGCCTGGTCTAAGCAAAAACATACAAC | |
| | eProPheCysLeuTrpArgAlaTrpSerLysGlnLysHisThrTh | |
| 2791 | AGACCTGGGTTTTCCTCGAAGTGCCCTTCCACCCTCCTGCCCGTA | |
| | rAspLeuGlyPheProArgSerAlaLeuProProSerCysProTy | |
| 2836 | TACTATGGTGCCATTGGGAGGACTCCCAGGCCACCAGGCAGTGGA | |
| | rThrMetValProLeuGlyGlyLeuProGlyHisGlnAlaValAs | |
| 2881 | CAGCCCTACCTCAGTGGCATCAGTGGACGGGCCTGTGCTAATGGG | |
| | pSerProThrSerValAlaSerValAspGlyProValLeuMetGl | |
| 2926 | ATCCACATGAATAGGGGCTGCCCCTCGGCTGCAGTGGGCTACCCG | |
| | ySerThr | |
| 2971 | GGCATGAAGCCCCAGCAGCACTGCCCAGGCGAGCTTCAGCAGCAG | |
| 3016 | AGTGACACCAGCAGCCTGCTGAGGCAGACCCATCTTGGCAATGGA | |
| 3061 | TATGACCCCCAAAGTCACCAGATCACGAGGGGTCCCAAGTCTAGC | |
| 3106 | CCGGACGAGGGCTCTTTCTTATACACACTGCCCGACGACTCCACT | |
| 3151 | CACCAGCTGCTGCAGCCCCATCACGACTGCTGCCAACGCCAGGAG | |
| 3196 | CAGCCTGCTGNTGTGGGCCAGTCAGGGGTGAGGAGAGCCCCCGAC | |
| 3241 | AGTCCTGTCCTGGAAGCAGTGTGGGACCCTCCATTTCACTCAGGG | |
| 3286 | CCCCCATGCTGCTTGGGCCTTGTGCCAGTTGAAGAGGTGGACAGT | |
| 3331 | CCTGACTCCTGCCAAGTGAGTGGAGGAGACTGGTGTCCCCAGCAC | |
| 3376 | CCCGTAGGGGCCTACGTAGGACAGGAACCTGGAATGCAGCTCTCC | |
| 3421 | CCGGGGCCACTGGTGCGTGTGTCTTTTGAAACACCACCTCTCACA | |
| 3466 | ATTTAGGCAGAAGCTGATATCCCAGAAAGACTATATATTGTTTTT | |
| 3511 | TTTTTAAAAAAAAAAAAAAAAAANCNCGGGGGGGGGCCCC | |

A search of the sequence databases using BLASTX reveals that the protein of clone 10354784.0.148 has 265 of 521 residues (50%) identical to, and 324 of 521 residues (62%) positive with, the 1256 residue CDO from Rattus norvegicus (ACC:NRDB O35158). It also has 258 of 514 residues (50%), and 320 of 514 residues (62%) positive with the 1240 residue human CDO protein (SPTREMBL:O1463 1). CDO is an oncogene-, serum-, and anchorage-regulated member of the Ig/fibronectin type III repeat family. The protein of clone 10354784.0.148 also has 607 of 616 residues (98%) identical to, and 609 of 616 residues (98%) positive with the 1053 residue human PRO1190 protein given in amino acid sequence SEQ ID NO:58 of PCT publication WO200012708-A2 (published Mar. 9, 2000).

The proteins of the invention encoded by clone 10354784.0.148 include the full protein disclosed as being encoded by the ORFs described herein, as well as any mature protein arising therefrom as a result of posttranslational modifications. Thus, the proteins of the invention encompass both a precursor and any active forms of the 10354784.0.148 protein.

In a further search of public sequence databases, POLY6 was found to have homology to the amino acid sequences shown in the BLASTP data listed in Table 29.

TABLE 29
BLASTP results for POLY6

| Gene Index/ | | Length | Identity | Positives | |
|---|---|---|---|---|---|
| Identifier | Protein/Organism | (aa) | (%) | (%) | Expect |
| REMTREMBL- | SEQUENCE 1 FROM PATENT | 739 | 730/739 | 732/739 | 0.0 |
| ACC:CAC43885 | WO0127277 - Homo sapiens | | (98%) | (99%) | |
| REMTREMBL- | SEQUENCE 3 FROM PATENT | 739 | 727/739 | 729/739 | 0.0 |
| ACC:CAC43886 | WO0127277 - Homo sapiens | | (98%) | (98%) | |
| SPTREMBL- | BROTHER OF CDO - Homo | 1114 | 690/698 | 691/698 | 0.0 |
| ACC:Q9BWV1 | sapiens | | (98%) | (98%) | |
| SPTREMBL- | BROTHER OF CDO - Mus | 1102 | 631/698 | 650/698 | 0.0 |
| ACC:Q923W7 | musculus | | (90%) | (93%) | |
| SPTREMBL- | BROTHER OF CDO - Xenopus | 1056 | 520/712 | 582/712 | 3.3e-273 |
| ACC:Q90Z03 | laevis | | (73%) | (81%) | |

The homology of these sequences is shown graphically in the ClustalW analysis shown in Table 29. In the ClustalW alignment of the POLY6 protein, the black outlined amino acid residues indicate regions of conserved sequence (i.e., regions that may be required to preserve structural or functional properties), whereas non-highlighted amino acid residues are less conserved and can potentially be mutated to a much broader extent without altering protein structure or function. POLY6 polypeptide is provided in lane 1.

[image]

[image]

BLAST analysis was performed on sequences from the Patp database, which is a proprietary database that contains sequences published in patents and patent publications. Patp results include those listed in Table 31.

TABLE 31
Patp BLASTP Analysis for POLY6

| Sequences | | | | | |
|---|---|---|---|---|---|
| producing | | | | | |
| High-scoring | | Length | Identity | Positive | |
| Segment Pairs | Protein/Organism | (aa) | (%) | (%) | E value |
| AAU00394 | Human secreted protein, | 735 | 733/735 | 733/735 | 0.0 |
| | POLY6-Homo sapiens | | (99%) | (99%) | |
| AAB62397 | Human MBSP1 polypeptide | 739 | 730/739 | 732/739 | 0.0 |
| | (clone 10354784.0.335)- | | (98%) | (99%) | |
| | Homo sapiens | | | | |
| AAB62398 | Human MBSP2 polypeptide | 739 | 727/739 | 729/739 | 0.0 |
| | (clone | | (98%) | (98%) | |
| | 10354784.0.335.S3347A)- | | | | |
| | Homo sapiens | | | | |
| AAU29243 | Human PRO polypeptide | 1115 | 690/698 | 692/698 | 0.0 |
| | sequence #220-Homo | | (98%) | (99%) | |
| | sapiens | | | | |
| AAY99357 | Human PRO1190 (UNQ604) | 1053 | 607/616 | 609/616 | 0.0 |
| | amino acid sequence SEQ ID | | (98%) | (98%) | |
| | NO: 58-Homo sapiens | | | | |

The presence of identifiable domains in POLY6, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results for POLY6 as disclosed in Tables 32, were collected from the Conserved Domain Database (CDD) with Reverse Position Specific BLAST analyses. This BLAST analysis software samples domains found in the Smart and Pfam collections.

Table 32 lists the domain description from DOMAIN analysis results against POLY6. This indicates that the POLY6 sequence has properties similar to those of other proteins known to contain these domains. For Table 32, fully conserved single residues are indicated by black shading or by the sign (|) and “strong” semi-conserved residues are indicated by grey shading or by the sign (+). In a sequence alignment herein, fully conserved single residues are calculated to determine percent homology, and conserved and “strong” semi-conserved residues are calculated to determine percent positives. The “strong” group of conserved amino acid residues may be any one of the following groups of amino acids: STA, NEQK, NHQK, NDEQ, QHRK, MILV, MILF, HY, FYW.

TABLE 32
Domain Analysis of POLY6

Parsed for domains:

| Model | Domain | seq-f | seq-t | hmm-f | hmm-t | score | E-value |
|---|---|---|---|---|---|---|---|
| ig | 1/2 | 44 | 100 | 1 | 45 | 33.3 | 8.8e−09 |
| ig | 2/2 | 136 | 192 | 1 | 45 | 41.7 | 2.3e−11 |
| fn3 | 1/3 | 270 | 361 | 1 | 84 | 37.0 | 4.3e−07 |
| fn3 | 2/3 | 406 | 493 | 1 | 84 | 59.0 | 1e−13 |
| fn3 | 3/3 | 512 | 602 | 1 | 84 | 28.0 | 0.00021 |
| Gemini_mov | 1/1 | 624 | 705 | 1 | 101 | −37.2 | 2.6 |

Alignments of top-scoring domains:

ig: domain 1 of 2, from 44 to 100: score 33.3, E = 8.8e−09

| | | | | |
|---|---|---|---|---|
| | | *->GesvtLtCsvsgfgpp.p.vtWlrngk............lsiti.sv | | |
| \|+\|+ \| \| +\| \| \|+\|+\|\|\| ++\| + ++ ++++ ++\|+\|+++ | | | | |
| POLY6A | 44 | GQSLILECVAS--GIPpPrVTWAKDGSsvtgynktrfllSNLLIdTT | 88 | |
| | | TpeDsgGtYtCvv | | (SEQ ID NO:66) |
| | | ++\|\|\| \|\|+\|+ | | |
| POLY6A | 89 | SEEDS-GTSRCMP | 100 | (SEQ ID NO:67) |

ig: domain 2 of 2, from 136 to 192: score 41.7, E = 2.3e−11

| | | | | |
|---|---|---|---|---|
| | | \|->GesvtLtCsvsgfgpp.p.vtWlrngk............lslti.sv | | |
| | | \|+\|++\|\|\|+\|+ \|+\|+\|+\| \|\|\|\| + ++++ + ++ \|++ \|+ | | |
| POLY6A | 136 | GQSAKLTCEVR--GNPpPsVLWLRNAVplissqrlrlsrRALRVlSM | 180 | |
| | | TpeDsgGtYtCvv | | (SEQ ID NO:68) |
| | | \|\|\|+ \|+\| \|++ | | |
| POLY6A | 181 | GPEDE-GVYQCMA | 192 | (SEQ ID NO:69) |

fn3: domain 1 of 3, from 270 to 361: score 37.0, E = 4.3e−07

| | | | | |
|---|---|---|---|---|
| | | *->P.saPtnltvtdv.tstsltlsWsppt...gngpitgYevtyR.qpk | | |
| | | \| \|\| \|+ ++ ++\| \| \| \| +++\|++\|\| +\| \|+ \|+\| + | | |
| POLY6A | 270 | PaEAPIILSSPRTsKTDSYELVWRPRHegsGRAPILYYVVKHRkQvT | 316 | |
| | | nggewnelt.vpgtttsytltgLkPgteYevrVqAvnggG.GpeS<-* | | (SEQ ID NO :70) |
| | | \|+ ++ +++++\| ++++ \|\|\| \| \|\|+ \|\|\| +\|+\| +\|+\|+ | | |
| POLY6A | 317 | NSSDDWTISgIPANQHRLTLTRLDPGSLYEVEMAAYNCAGeGQTA | 361 | (SEQ ID NO:71) |

fn3: domain 2 of 3, from 406 to 493: score 59.0, E = 1e−13

| | | | | |
|---|---|---|---|---|
| | | *->PsaPtnltvtdvtstsltlsWsppt.gngpitgYevtyRqpkngge. | | |
| | | \| \|\| ++\|++ ++\|\|+ ++\| \| +\|+ \|\|++++\|+\| ++ + ++ | | |
| POLY6A | 406 | PEAPDRPTISTASETSVYVTWIPRGnGGFPIQSFRVEY-KKLKKVGd | 451 | |
| | | ..wneltvpgtttsytltgLkPgteYevrVqAvnggG.GpeS<-* | | (SEQ ID NO:72) |
| | | + +\|++ \|+++\|\|\|+ \| \|++\|\|+\|+\| \|++++\| | | |
| POLY6A | 452 | wiLATSAIPPSRLSVEITGLEKGASYKFRVRALMMLGeSEpS | 493 | (SEQ ID NO:73) |

fn3: domain 3 of 3, from 512 to 602: score 28.0, E = 0.00021

| | | | | |
|---|---|---|---|---|
| | | *->PsaPtnltvtdv.tstsltlsWsppt...gngpitgYevtyRqpkng | | |
| | | \| \| +\|+\|\| ++\|++ \| \| + +++\| \|\| \|+ + \|\| \|+++ | | |
| POLY6A | 512 | PVAGPYITFTDAvNETTIMLKWMYIPasnNNTPIHGFYIYYR-PTDS | 557 | |
| | | ge...wneltvpgtttsytltgLkPgteYevrVqAvnggG.GpeS<-* | | (SEQ ID NO:74) |
| | | +++ + +\| \|+++ + ++ \|+\| \| \| ++ \| \|+\|\|++++\| | | |
| POLY6A | 558 | DNdsdYKKDMVEGDKYWHSISHLQPETSYDIKMQCFNEGGeSEFS | 602 | (SEQ ID NO:75) |

Gemini_may: domain 1 of 1, from 624 to 705: score −37.2, E = 2.6

| | | | | |
|---|---|---|---|---|
| | | *->MdpagyqvpPsalpysqtPrvPtaaPssqKAGESSglpWSRVGeia. | | |
| | | \| +++ \| \|++++\| \|\|++\|+ \|\|++++ | | |
| POLY6A | 624 | --PTLAPPQPPLPETIERPVGTGAMVAR-----SSDVPYLIVGVVLg | 663 | |
| | | ..IftLVAVl..vlYLLYtWVLkDLILvlKAkrGRtTEElgFGptearSA | | |
| | | + +++ \| ++ +\|+ +++ +++ \|+ \|\|\| ++++ | | |
| POLYGA | 664 | siVLIIVTFIpfCLWRAWS-------------KQKHTTDLGFPRSALP-- | 698 | |
| | | LArsgavpp<-* | | (SEQ ID NO:76) |
| | | +\| + + | | |
| POLY6A | 699 | --PSCPYTM | 705 | (SEQ ID NO:77) |

CDO is an ocogene-, serum-, and anchorage-regulated member of the Ig/fibronectin type III repeat family.

POLY7

A POLY7 nucleic acid according to the invention includes the nucleic acid sequence represented in Clone13043743.0.15. RNA sequences homologous to this clone are found in the pituitary gland and osteosarcoma. A representation of the nucleotide sequence of clone 13043743.0.15 is given in Table 8. This clone includes a nucleotide sequence (SEQ ID NO:13) of 971 bp. This nucleotide sequence has an open reading frame encoding a polypeptide of 146 amino acid residues (represented in Table 8; SEQ ID NO:14) with a predicted molecular weight of 16220 Da. The start codon is at nucleotides 471-473 and the stop codon is at nucleotides 909-91 1. The protein of SEQ ID NO:14 is predicted by the PSORT program to localize in the plasma membrane with a certainty of 0.7000. The programs PSORT and SignalP predict that there most likely is no signal peptide, but there is a slight probability that a cleavage site occurs between residues 42 and 43: TQQ-QW.

TABLE 8
Translated Protein - Frame: 3 - Nucleotide 471 to 908

| | | |
|---|---|---|
| 1 | AAATTCTGGCCGATTTATCCCGAGAACAATGTAAGTGGAAACTAA | (SEQ ID NO:13) |
| 46 | CAATGAAGATGTTCATTTGAGCCAACAGAGGCCTGTTTACTTCCT | |
| 91 | AAAATAATCTCTTAGTTACATAACCATAAGATGTCCTAAGTTGAG | |
| 136 | TCCTGTTTGCTTCTTTGTAGCTTTTGGTGAAGGCCCTAGAGTATC | |
| 181 | TGCTTATGTCCTGACAACATGGGGATTCTCTGGGACAAGGCAAGA | |
| 226 | GGACCCAAGCTGTGTTCAAATGAGACCCAGAGATCCCAGTGCAAA | |
| 271 | GGGACACCTTTGGTAAAACTGGCCACTTTGGGTCATATAGATCCC | |
| 316 | AAAAGAAATCATGCCTGACTTCCTAAAATCAAAACCATAGGGATT | |
| 361 | TATCAAGAAGAAACCCTGTTTAATTGGCCTATAGATGAGGATGCA | |
| 406 | ACCTAAGGCATGACTGTTGCTGAATGAGCCCAAGTGATGGGCCAA | |
| 451 | AAAGGTGAACCAGTCCAAACATGGCAGGACCAAGAAGGAACAAGG | |
| | MetAlaGlyProArgArgAsnLysV | (SEQ ID NO:14) |
| 496 | TAGGAGAGGGCAGAGGGGTGGAGGGCAGAGAGGTGGAGGGGAGGA | |
| | alGlyGluGlyArgGlyValGluGlyArgGluValGluGlyArgS | |
| 541 | GTGCCTGCATGGCCATGGGATTGTTCTTCATTCCCTTTCTCAACT | |
| | erAlaCysMetAlaMetGlyLeuPhePheIleProPheLeuAsnC | |
| 586 | GCACCCAGCAGCAGTGGTTTTTGCTAGGCCTTTTGAAGACAGCAG | |
| | ysThrGlnGlnGlnTrpPheLeuLeuGlyLeuLeuLysThrAlaG | |
| 631 | GAATCTGGGAGAAGGAACATCATCGTCTTTCACAGCATGGAAACA | |
| | lyIleTrpGluLysGluHisHisArgLeuSerGlnHisGlyAsnI | |
| 676 | TCAATCTTATTCCAGAGAAGGGAAGAAGTCCCCAAAGGTATGTCC | |
| | leAsnLeuIleProGluLysGlyArgSerProGlnArgTyrValA | |
| 721 | GGTTTAACAGTTTCTCAAGTGGGCCAGGAAGTTCTTTTTCATGTT | |
| | rgPheAsnSerPheSerSerGlyProGlySerSerPheSerCysS | |
| 766 | CTGGGCTCAATCGTGATGCTTTGATTTCACTTGGTATTTTACTTT | |
| | erGlyLeuAsnArgAspAlaLeuIleSerLeuGlyIleLeuLeuL | |
| 811 | TAGTTTTGTCTCTAACATCTGGAGCAAAGATCAGAAGACCTGAGT | |
| | euValLeuSerLeuThrSerGlyAlaLysIleArgArgProGluP | |
| 856 | TCCAGATTTATTCTGTGACTCAATCACTGCTTCAATCACTGAGGG | |
| | heGlnIleTyrSerValThrGlnSerLeuLeuGlnSerLeuArgA | |
| 901 | ACGTGGTGTGATGTTCTTTGCTCTGAGCTTCTGCTTCTTGATCTA | |
| | spValVal | |
| 946 | AAAAGACAGGTGGTTGTTCTGGGTGA | |

A search of the sequence databases using BLASTP reveals that the protein of clone 13043743.0.15 has 14 of 43 residues (32%) identical to, and 21 of 43 residues (48%) positive with a 64 residue fragment of the MHC CLASS II alpha chain proximal membrane peptide from Scyliorhinus canicula (spotted dogfish) (ACC:002933).

The proteins of the invention encoded by clone 13043743.0.15 include the full protein disclosed as being encoded by the ORFs described herein, as well as any mature protein arising therefrom as a result of posttranslational modifications. Thus, the proteins of the invention encompass both a precursor and any active forms of the 13043743.0.15 protein.

POLY8

A POLY8 nucleic acid according to the invention includes the nucleic acid sequence represented in Clone 16532807.0.137. RNA sequences homologous to this clone are found in the testis. A representation of the nucleotide sequence of clone 16532807.0.137 is given in Table 9. This clone includes a nucleotide sequence (SEQ ID NO:15) of 3670 bp. This nucleotide sequence has an open reading frame encoding a polypeptide of 1120 amino acid residues (represented in Table 9; SEQ ID NO:16) with a predicted molecular weight of 122733.8 Da. The start codon is at nucleotides 113-115 and the stop codon is at nucleotides 3473-3745. The protein of SEQ ID NO:16 is predicted by the PSORT program to localize in the plasma membrane with a certainty of 0.7000. The programs PSORT and SignalP predict that there most likely is no signal peptide.

TABLE 9
Translated Protein - Frame: 2 - Nucleotide 113 to 3472

| | | |
|---|---|---|
| 1 | CAATTTTGACTGTCTTCATCAAAACGATGTGTCTGTGATCTGCTC | (SEQ ID NO:15) |
| 46 | AGACCAGTTGCTATTCAAACTCTGGAACTTCTGGAACTATTTGCC | |
| 91 | TCTGGAAGAGTAAGGAAGGGCCATGAAGAATGATGCAGATGTGAA | |
| | MetLysAsnAspAlaAspValLy | (SEQ ID NO:16) |
| 136 | AATAAGGTTGGAAAACCGTCCCAACAGTGAAAACAGATGTGTATG | |
| | SIleArgLeuGluAsnArgProAsnSerGluAsnArgCysValTr | |
| 181 | GCCAACCCACAGGAAAATGGATGGAGCAGATTTGGAACTGCGACT | |
| | pProThrHisArgLysMetAspGlyAlaAspLeuGluLeuArgLe | |
| 226 | AGCAGATGGAAGTAACAATTGTTCAGGAAGAGTAGAGGTGAGAAT | |
| | uAlaAspGlySerAsnAsnCysSerGlyArgValGluValArgIl | |
| 271 | TCATGAACAGTGGTGGACAATATGTGACCAGAACTGGAAGAATGA | |
| | eHisGluGlnTrpTrpThrIleCysAspGlnAsnTrpLysAsnGl | |
| 316 | ACAAGCCCTTGTGGTTTGTAAGCAGCTAGGATGTCCGTTCAGCGT | |
| | uGlnAlaLeuValValCysLysGlnLeuGlyCysProPheSerVa | |
| 361 | CTTTGGCAGTCGTCGTGCTAAACCTAGTAATGAAGCTAGAGACAT | |
| | lPheGlySerArgArgAlaLysProSerAsnGluAlaArgAspIl | |
| 406 | TTGGATAAACAGCATATCTTGCACTGGGAATGAGTCAGCTCTCTG | |
| | eTrpIleAsnSerIleSerCysThrGlyAsnGluSerAlaLeuTr | |
| 451 | GGACTGCACATATGATGGAAAAGCAAAGCGAACATGCTTCCGAAG | |
| | pAspCysThrTyrAspGlyLysAlaLysArgThrCysPheArgAr | |
| 496 | ATCAGATGCTGGAGTAATTTGTTCTGATAAGGCAGATCTGGACCT | |
| | gSerAspAlaGlyValIleCysSerAspLysAlaAspLeuAspLe | |
| 541 | AAGGCTTGTCGGGGCTCATAGCCCCTGTTATGGGAGATTGGAGGT | |
| | uArgLeuValGlyAlaHisSerProCysTyrGlyArgLeuGluVa | |
| 586 | GAAATACCAAGGAGAGTGGGGGACTGTGTGTCATGACAGATGGAG | |
| | lLysTyrGlnGlyGluTrpGlyThrValCysHisAspArgTrpSe | |
| 631 | CACAAGGAATGCAGCTGTTGTGTGTAAACAATTGGGATGTGGAAA | |
| | rThrArgAsnAlaAlaValValCysLysGlnLeuGlyCysGlyLy | |
| 676 | GCCTATGCATGTGTTTGGTATGACCTATTTTAAAGAAGCATCAGG | |
| | sProMetHisValPheGlyMetThrTyrPheLysGluAlaSerGl | |
| 721 | ACCTATTTGGCTGGATGACGTTTCTTGCATTGGAAATGAGTCAAA | |
| | yProIleTrpLeuAspAspValSerCysIleGlyAsnGluSerAs | |
| 766 | TATCTGGGACTGTGAACACAGTGGATGGGGAAAGCATAATTGTGT | |
| | nIleTrpAspCysGluHisSerGlyTrpGlyLysHisAsnCysVa | |
| 811 | ACACAGAGAGGATGTGATTGTAACCTGCTCAGGTGATGCAACATG | |
| | iHisArgGluAspValIleValThrCysSerGlyAspAlaThrTr | |
| 856 | GGGCCTGAGGCTGGTGGGCGGCAGCAACCGCTGCTCGGGAAGACT | |
| | pGlyLeuArgLeuValGlyGlySerAsnArgCysSerGlyArgLe | |
| 901 | GGAGGTGTACTTTCAAGGACGGTGGGGCACAGTGTGTGATGACGG | |
| | uGluValTyrPheGlnGlyArgTrpGlyThrValCysAspAspGl | |
| 946 | CTGGAACGGTAAAGCTGCAGCTGTGGTGTGTAGCCAGCTGGACTG | |
| | yTrpAsnGlyLysAlaAlaAlaValValCysSerGlnLeuAspCy | |
| 991 | CCCATCTTCTATCATTGGCATGGGTCTGGGAAACGCTTCTACAGG | |
| | sProSerSerIleIleGlyMetGlyLeuGlyAsnAlaSerThrGl | |
| 1036 | ATATGGAAAAATTTGGCTCGATGATGTTTCCTGTGATGGAGATGA | |
| | yTyrGlyLysIleTrpLeuAspAspValSerCysAspGlyAspGl | |
| 1081 | GTCAGATCTCTGGTCATGCAGGAACAGTGGGTGGGGAAATAATGA | |
| | uSerAspLeuTrpSerCysArgAsnSerGlyTrpGlyAsnAsnAs | |
| 1126 | CTGCAGTCACAGTGAAGATGTTGGAGTGATCTGTTCTGATGCATC | |
| | pCysSerHisSerGluAspValGlyValIleCysSerAspAlaSe | |
| 1171 | GGATATGGAGCTGAGGCTTGTGGGTGGAAGCAGCAGGTGTGCTGG | |
| | rAspMetGluLeuArgLeuValGlyGlySerSerArgCysAlaGl | |
| 1216 | AAAAGTTGAGGTGAATGTCCAGGGTGCCGTGGGAATTCTGTGTGC | |
| | yLysValGluValAsnValGlnGlyAlaValGlyIleLeuCysAl | |
| 1261 | TAATGGCTGGGGAATGAACATTGCTGAAGTTGTTTGCAGGCAACT | |
| | aAsnGlyTrpGlyMetAsnIleAlaGluValValCysArgGlnLe | |
| 1306 | TGAATGTGGGTCTGCAATCAGGGTCTCCAGAGAGCCTCATTTCAC | |
| | uGluCysGlySerAlaIleArgValSerArgGluProHisPheTh | |
| 1351 | AGAAAGAACATTACACATCTTAATGTCGAATTCTGGCTGCACTGG | |
| | rGluArgThrLeuHisIleLeuMetSerAsnSerGlyCysThrGl | |
| 1396 | AGGGGAAGCCTCTCTCTGGGATTGTATACGATGGGAGTGGAAACA | |
| | yGlyGluAlaSerLeuTrpAspCysIleArgTrpGluTrpLysGl | |
| 1441 | GACTGCGTGTCATTTAAATATGGAAGCAAGTTTGATCTGCTCAGC | |
| | nThrAlaCysHisLeuAsnMetGluAlaSerLeuIleCysSerAl | |
| 1486 | CCACAGGCAGCCCAGGCTGGTTGGAGCTGATATGCCCTGCTCTGG | |
| | aHisArgGlnProArgLeuValGlyAlaAspMetProCysSerGl | |
| 1531 | ACGTGTTGAAGTGAAACATGCAGACACATGGCGCTCTGTCTGTGA | |
| | yArgValGluValLysHisAlaAspThrTrpArgSerValCysAs | |
| 1576 TTCTGATTTCTCTCTTCATGCTGCCAATGTGCTGTGCAGAGAATT | | |
| | pSerAspPheSerLeuHisAlaAlaAsnValLeuCysArgGluLe | |
| 1621 | AAACTGTGGAGATGCCATATCTCTTTCTGTGGGAGATCACTTTGG | |
| | uAsnCysGlyAspAlaIleSerLeuSerValGlyAspHisPheGl | |
| 1666 | AAAAGGGAATGGTCTAACTTGGGCCGAAAAGTTCCAGTGTGAAGG | |
| | yLysGlyAsnGlyLeuThrTrpAlaGluLysPheGlnCysGluGl | |
| 1711 | GAGTGAAACTCACCTTGCATTATGCCCCATTGTTCAACATCCGGA | |
| | ySerGluThrHisLeuAlaLeuCysProIleValGlnHisProGl | |
| 1756 | AGACACTTGTATCCACAGCAGAGAAGTTGGAGTTGTCTGTTCCCG | |
| | uAspThrCysIleHisSerArgGluValGlyValValCysSerAr | |
| 1801 | ATATACAGATGTCCGACTTGTGAATGGCAAATCCCAGTGTGACGG | |
| | gTyrThrAspValArgLeuValAsnGlyLysSerGlnCysAspGl | |
| 1846 | GCAAGTGGAGATCAACGTGCTTGGACACTGGGGCTCACTGTGTGA | |
| | yGlnValGluIleAsnValLeuGlyHisTrpGlySerLeuCysAs | |
| 1891 | CACCCACTGGGACCCAGAAGATGCCCGTGTTCTATGCAGACAGCT | |
| | pThrHisTrpAspProGluAspAlaArgValLeuCysArgGlnLe | |
| 1936 | CAGCTGTGGGACTGCTCTCTCAACCACAGGAGGAAAATATATTGG | |
| | uSerCysGlyThrAlaLeuSerThrThrGlyGlyLysTyrIleGl | |
| 1981 | AGAAAGAAGTGTTCGTGTGTGGGGACACAGGTTTCATTGCTTAGG | |
| | yGluArgSerValArgValTrpGlyHisArgPheHisCysLeuGL | |
| 2026 | GAATGAGTCACTTCTGGATAACTGTCAAATGACAGTTCTTGGAGC | |
| | yAsnGluSerLeuLeuAspAsnCysGlnMetThrValLeuGlyAl | |
| 2071 | ACCTCCCTGTATCCATGGAAATACTGTCTCTGTGATCTGCACAGG | |
| | aProProCysIleHisGlyAsnThrValSerValIleCysThrGl | |
| 2116 | AAGCCTGACCCAGCCACTGTTTCCATGCCTCGCAAATGTATCTGA | |
| | ySerLeuThrGlnProLeuPheProCysLeuAlaAsnValSerAs | |
| 2161 | CCCATATTTGTCTGCAGTTCCAGAGGGCAGTGCTTTGATCTGCTT | |
| | pProTyrLeuSerAlaValProGluGlySerAlaLeuIleCysLe | |
| 2206 | AGAGGACAAACGGCTCCGCCTAGTGGATGGGGACAGCCGCTGTGC | |
| | uGluAspLysArgLeuArgLeuValAspGlyAspSerArgCysAl | |
| 2251 | CGGGAGAGTAGAGATCTATCACGACGGCTTCTGGGGCACCATCTG | |
| | aGlyArgValGluIleTyrHisAspGlyPheTrpGlyThrIleCy | |
| 2296 | TGATGACGGCTGGGACCTGAGCGATGCCCACGTGGTGTGTCAAAA | |
| | sAspAspGlyTrpAspLeuSerAspAlaHisValValCysGlnLy | |
| 2341 | GCTGGGCTGTGGAGTGGCCTTCAATGCCACGGTCTCTGCTCACTT | |
| | sLeuGlyCysGlyValAlaPheAsnAlaThrValSerAlaHisPh | |
| 2386 | TGGGGAGGGGTCAGGGCCCATCTGGCTGGATGACCTGAACTGCAC | |
| | eGlyGluGlySerGlyProIleTrpLeuAspAspLeuAsnCysTh | |
| 2431 | AGGAATGGAGTCCCACTTGTGGCAGTGCCCTTCCCGCGGCTGGGG | |
| | rGlyMetGluSerHisLeuTrpGlnCysProSerArgGlyTrpGl | |
| 2476 | GCAGCACGACTGCAGGCACAAGGAGGACGCAGGGGTCATCTGCTC | |
| | yGlnHisAspCysArgHisLysGluAspAlaGlyValIleCysSe | |
| 2521 | AGAATTCACAGCCTTGAGGCTCTACAGTGAAACTGAAACAGGGAG | |
| | rGluPheThrAlaLeuArgLeuTyrSerGluThrGluThrGlySe | |
| 2566 | CTGTGCTGGGAGATTGGAAGTCTTCTATAACGGGACCTGGGGCAG | |
| | rCysAlaGlyArgLeuGluValPheTyrAsnGlyThrTrpGlySe | |
| 2611 | CGTCGGCAGGAGGAACATCACCACAGCCATAGCAGGCATTGTGTG | |
| | rValGlyArgArgAsnIleThrThrAlaIleAlaGlyIleValCy | |
| 2656 | CAGGCAGCTGGGCTGTGGGGAGAATGGAGTTGTCAGCCTCGCCCC | |
| | sArgGlnLeuGlyCysGlyGluAsnGlyValValSerLeuAlaPr | |
| 2701 | TTTATCTAAGACAGGCTCTGGTTTCATGTGGGTGGATGACATTCA | |
| | oLeuSerLysThrGlySerGlyPheMetTrpValAspAspIleGL | |
| 2746 | GTGTCCTAAAACGCATATCTCCATATGGCAGTGCCTGTCTGCCCC | |
| | nCysProLysThrHisIleSerIleTrpGlnCysLeuSerAlaPr | |
| 2791 | ATGGGAGCGAAGAATCTCCAGCCCAGCAGAAGAGACCTGGATCAC | |
| | oTrpGluArgArgIleSerSerProAlaGluGluThrTrpIleTh | |
| 2836 | ATGTGAAGATAGAATAAGAGTGCGTGGAGGAGACACCGAGTGCTC | |
| | rCysGluAspArgIleArgValArgGlyGlyAspThrGluCysSe | |
| 2881 | TGGGAGAGTGGAGATCTGGCACGCAGGCTCCTGGGGCACAGTGTG | |
| | rGlyArgValGluIleTrpHisAlaGlySerTrpGlyThrValCy | |
| 2926 | TGATGACTCCTGGGACCTGGCCGAGGCGGAAGTGGTGTGTCAGCA | |
| | sAspAspSerTrpAspLeuAlaGluAlaGluValValCysGlnGl | |
| 2971 | GCTGGGCTGTGGCTCTGCTCTGGCTGCCCTGAGGGACGCTTCGTT | |
| | nLeuGlyCysGlySerAlaLeuAlaAlaLeuArgAspAlaSerPh | |
| 3016 | TGGCCAGGGAACTGGAACCATCTGGTTGGATGACATGCGGTGCAA | |
| | eGlyGlnGlyThrGlyThrIleTrpLeuAspAspMetArgCysLy | |
| 3061 | AGGAAATGAGTCATTTCTATGGGACTGTCACGCCAAACCCTGGGG | |
| | sGlyAsnGluSerPheLeuTrpAspCysHisAlaLysProTrpGl | |
| 3106 | ACAGAGTGACTGTGGACACAAGGAAGATGCTGGCGTGAGGTGCTC | |
| | yGlnSerAspCysGlyHisLysGluAspAlaGlyValArgCysSe | |
| 3151 | TGGACAGTCGCTGAAATCACTGAATGCCTCCTCAGGTCATTTAGC | |
| | rGlyGlnSerLeuLysSerLeuAsnAlaSerSerGlyHisLeuAl | |
| 3196 | ACTTATTTTATCCAGTATCTTTGGGCTCCTTCTCCTGGTTCTGTT | |
| | aLeuIleLeuSerSerIlePheGlyLeuLeuLeuLeuValLeuPh | |
| 3241 | TATTCTATTTCTCACGTGGTGCCGAGTTCAGAAACAAAAACATCT | |
| | eIleLeuPheLeuThrTrpCysArgValGlnLysGlnLysHisLe | |
| 3286 | GCCCCTCAGAGTTTCAACCAGAAGGAGGGGTTCTCTCGAGGAGAA | |
| | uProLeuArgValSerThrArgArgArgGlySerLeuGluGluAs | |
| 3331 | TTTATTCCATGAGATGGAGACCTGCCTCAAGAGAGAGGACCCACA | |
| | nLeuPheHisGluMetGluThrCysLeuLysArgGluAspProHi | |
| 3376 | TGGGACAAGAACCTCAGATGACACCCCCAACCATGGTTGTGAAGA | |
| | sGlyThrArgThrSerAspAspThrProAsnHisGlyCysGluAs | |
| 3421 | TGCTAGCGACACATCGCTGTTGGGAGTTCTTCCTGCCTCTGAAGC | |
| | pAlaSerAspThrSerLeuLeuGlyValLeuProAlaSerGluAl | |
| 3466 | CACAAAATGACTTTAGACTTCCAGGGCTCACCAGCTCAACCTCTA | |
| | aThrLys | |
| 3511 | AATATCTTTGAAGGAGACAACAACTTTTAAATGAATAAAGAGGAA | |
| 3556 | GTCAAGTTGCCCTATGGAAAACTTGTCCAAATAACATTTCTTGAA | |
| 3601 | CAATAGGAGAACAGCTAAATTGATAAAGACTGGTGATAATAAAAA | |
| 3646 | TTGAATTATGTATATCCCTGTTAAA | |

A search of the sequence databases using BLASTP reveals that the protein of clone 16532807.0.137 has 606 of 1099 residues (55%)identical to, and 773 of 1099 residues (70%) positive with, the 1151 residue human M130 antigen, cytoplasmic variant 1 precursor (ACC:Q07899).

The proteins of the invention encoded by clone 16532807.0.137 include the fall protein disclosed as being encoded by the ORFs described herein, as well as any mature protein arising therefrom as a result of posttranslational modifications. Thus, the proteins of the invention encompass both a precursor and any active forms of the 16532807.0.137 protein.

POLY9

A POLY9 nucleic acid according to the invention includes the nucleic acid sequence represented in Clone 17883252.0.13. RNA sequences homologous to this clone are found in the thyroid gland, fetal kidney, kidney, fetal lung, lymph node, and placenta. A representation of the nucleotide sequence of clone 17883252.0.13 is given in Table 10. This clone includes a nucleotide sequence (SEQ ID NO:17) of 1619 bp. This nucleotide sequence has an open reading frame encoding a polypeptide of 234 amino acid residues (represented in Table 10; SEQ ID NO:18) with a predicted molecular weight of 25396.6 Da. The start codon is at nucleotides 514-516 and the stop codon is at nucleotides 1216-1218. The protein of SEQ ID NO:18 is predicted by the PSORT program to localize in the endoplasmic reticulum (membrane) with a certainty of 0.5500, and that the protein may have an uncleavable N-terminal signal sequence. The program SignalP predicts that there is a signal peptide with the most likely cleavage site between residues 22 and 23: TEH-AY.

TABLE 10
Translated Protein - Frame: 1 - Nucleotide 514 to 1215

| | | |
|---|---|---|
| 1 | GATTCCAGAAGGCTGAGGCCGGCAGGTCCCTGGTGGGGCTGGATC | (SEQ ID NO:17) |
| 46 | CCAGCTCTGCCCACGAACACCCCGTTTGCATAGACTGGGGTGCAA | |
| 91 | ACTCACCCCTGCCCTGCTGTGAGGAGGGCCCTGGAACCAGGCAGG | |
| 136 | ACAGGAGAAACGGCCACCCGTAGCTGGAGCCATCCTTCCCGGAGC | |
| 181 | CTCGGGCAGATGCCCAGCAGGATCCACTCGATTCCTGCACCAAGA | |
| 226 | GCTCTGGACAGCGTCACCCCCCCTGTGCCCCCCAGGCTGTGGCCC | |
| 271 | CAGCTGTTTGTGCCTGGCGAGGGTCTGGCTAGCTGGAAGAGGGGG | |
| 316 | CCAGCGGAGGAGAGAGTGGGCGCCACCGTGGGGCTGTCCCACCGG | |
| 361 | TGGAGGCTCCAGCGGAGATGAGCTGGGCAGGCCTCGCGGAGCAAG | |
| 406 | TGCAAACTGCACCCGCGTCCTGGGGGCATCTGCGGGGAGACTTAG | |
| 451 | GGGTCATGCTTTGTGCCCCAGGCCACCCAGAGGAGAAGGCCACCC | |
| 496 | CGCCTGGAGGCACAGGCCATGAGGGGCTCTCAGGAGGTGCTGCTG | |
| | MetArgGlySerGlnGluValLeuLeu | (SEQ ID NO:18) |
| 541 | ATGTGGCTTCTGGTGTTGGCAGTGGGCGGCACAGAGCACGCCTAC | |
| | MetTrpLeuLeuValLeuAlaValGlyGlyThrGluHisAlaTyr | |
| 586 | CGGCCCGGCCGTAGGGTGTGTGCTGTCCGGGCTCACGGGGACCCT | |
| | ArgProGlyArgArgValCysAlaValArgAlaHisGlyAspPro | |
| 631 | GTCTCCGAGTCGTTCGTGCAGCGTGTGTACCAGCCCTTCCTCACC | |
| | ValSerGluSerPheValGlnArgValTyrGlnProPheLeuThr | |
| 676 | ACCTGCGACGGGCACCGGGCCTGCAGCACCTACGCAATATGCCAG | |
| | ThrCysAspGlyHisArgAlaCysSerThrTyrAlaIleCysGln | |
| 721 | CCGCCATGCCGGAACGGAGGGAGCTGTGTCCAGCCTGGCCGCTGC | |
| | ProProCysArgAsnGlyGlySerCysValGlnProGlyArgCys | |
| 766 | CGCTGCCCTGCAGGATGGCGGGGTGACACTTGCCAGTCAGATGTG | |
| | ArgCysProAlaGlyTrpArgGlyAspThrCysGlnSerAspVal | |
| 811 | GATGAATGCAGTGCTAGGAGGGGCGGCTGTCCCCAGCGCTGCGTC | |
| | AspGluCysSerAlaArgArgGlyGlyCysProGlnArgCysVal | |
| 856 | AACACCGCCGGCAGTTACTGGTGCCAGTGTTGGGAGGGGCACAGC | |
| | AsnThrAlaGlySerTyrTrpCysGlnCysTrpGluGlyHisSer | |
| 901 | CTGTCTGCAGACGGTACACTCTGTGTGCCCAAGGGAGGGCCCCCC | |
| | LeuSerAlaAspGlyThrLeuCysValProLysGlyGlyProPro | |
| 946 | AGGGTGGCCCCCAACCCGACAGGAGTGGACAGTGCAATGAAGGAA | |
| | ArgValAlaProAsnProThrGlyValAspSerAlaMetLysGlu | |
| 991 | GAAGTGCAGAGGCTGCAGTCCAGGGTGGACCTGCTGGAGGAGAAG | |
| | GluValGlnArgLeuGlnSerArgValAspLeuLeuGluGluLys | |
| 1036 | CTGCAGCTGGTGCTGGCCCCACTGCACAGCCTGGCCTCGCAGGCA | |
| | LeuGlnLeuValLeuAlaProLeuHisSerLeuAlaSerGlnAla | |
| 1081 | CTGGAGCATGGGCTCCCGGACCCCGGCAGCCTCCTGGTGCACTCC | |
| | LeuGluHisGlyLeuProAspProGlySerLeuLeuValHisSer | |
| 1126 | TTCCAGCAGCTCGGCCGCATCGACTCCCTGAGCGAGCAGATTTCC | |
| | PheGlnGlnLeuGlyArgIleAspSerLeuSerGluGlnIleSer | |
| 1171 | TTCCTGGAGGAGCAGCTGGGGTCCTGCTCCTGCAAGAAAGACTCG | |
| | PheLeuGluGluGlnLeuGlySerCysSerCysLysLysAspSer | |
| 1216 | TGACTGCCCAGCGCCCCAGGCTGGACTGAGCCCCTCACGCCGCCC | |
| 1261 | TGCAGCCCCCATGCCCCTGCCCAACATGCTGGGGGTCCAGAAGCC | |
| 1306 | ACCTCGGGGTGACTGAGCGGAAGGCCAGGCAGGGCCTTCCTCCTC | |
| 1351 | TTCCTCCTCCCCTTCCTCGGGAGGCTCCCCAGACCCTGGCATGGG | |
| 1396 | ATGGGCTGGGATCTTCTCTGTGAATCCACCCCTGGCTACCCCCAC | |
| 1441 | CCTGGCTACCCCAACGGCATCCCAAGGCCAGGTGGGCCCTCAGCT | |
| 1486 | GAGGGAAGGTACGAGCTCCCTGCTGGAGCCTGGGACCCATGGCAC | |
| 1531 | AGGCCAGGCAGCCCGGAGGCTGGGTGGGGCCTCAGTGGGGCTGCT | |
| 1576 | GCCTGACCCCCAGCACAATAAAAATGAAACGTGAGCTGCAAAAA | |

A search of the sequence databases using BLASTP reveals that the protein of clone 17883252.0.13 has 171 of 174 residues (98%) identical and X of X (XX%) positive with the 273 residue human neuro-growth factor-like protein Zneu1 (ACC:W88381).

The proteins of the invention encoded by clone 17883252.0.13 include the full protein disclosed as being encoded by the ORFs described herein, as well as any mature protein arising therefrom as a result of posttranslational modifications. Thus, the proteins of the invention encompass both a precursor and any active forms of the 17883252.0.13 protein.

POLY10

A POLY10 nucleic acid according to the invention includes the nucleic acid sequence represented in Clone 17941787.0.3. RNA sequences homologous to this clone are found in the mammary gland and pituitary gland. A representation of the nucleotide sequence of clone 17941787.0.3 is given in Table 11. This clone includes a nucleotide sequence (SEQ ID NO:19) of 1441 bp. This nucleotide sequence has an open reading frame encoding a polypeptide of 430 amino acid residues (represented in Table11; SEQ ID NO:20) with a predicted molecular weight of 48793 Da. The start codon is at nucleotides 120-122 and the stop codon is at nucleotides 1410-1412. The protein of SEQ ID NO:20 is predicted by the PSORT program to localize extracellularly with a certainty of 0.3700, and to have a cleavable N-terminal signal sequence. The program SignalP predicts that there is a signal peptide with the most likely cleavage site between residues 27 and 28: VYA-CG.

TABLE 11
Translated Protein - Frame: 3 - Nucleotide 120 to 1409

| | | |
|---|---|---|
| 1 | CGCCGGTGGCTCGGCGGCGGCGGCGGCGGCGGCGGCGGCGGCGGC | (SEQ ID NO:19) |
| 46 | GGCGGCGTCGTCTACCTCCAGCTTCTCCTCCCTCCTCCTCCGTCT | |
| 91 | CCTCCTCTCTCTCCATCTGCTGTGGTTATGGCCTGTCGCTGGA | |
| | MetAlaCysArgTrpS | (SEQ ID NO:20) |
| 136 | GCACAAAAGAGTCTCCGCGGTGGAGGTCTGCGTTGCTCTTGCTTT | |
| | erThrLysGluSerProArgTrpArgSerAlaLeuLeuLeuLeuP | |
| 181 | TCCTCGCTGGGGTGTACGCTTGTGGAGAGACTCCAGAGCAAATAC | |
| | heLeuAlaGlyValTyrAlaCysGlyGluThrProGluGlnIleA | |
| 226 | GAGCACCAAGTGGCATAATCACAAGCCCAGGCTGGCCTTCTGAAT | |
| | rgAlaProSerGlyIleIleThrSerProGlyTrpProSerGluT | |
| 271 | ATCCTGCAAAAATCAACTGTAGCTGGTTCATAAGGGCAAACCCAG | |
| | yrProAlaLysIleAsnCysSerTrpPheIleArgAlaAsnProG | |
| 316 | GCGAAATCATTACTATAAGTTTTCAGGATTTTGATATTCAAGGAT | |
| | lyGluIleIleThrIleSerPheGlnAspPheAspIleGlnGlyS | |
| 361 | CCAGAAGGTGCAATTTGGACTGGTTGACAATAGAAACATACAAGA | |
| | erArgArgCysAsnLeuAspTrpLeuThrIleGluThrTyrLysA | |
| 406 | ATATTGAAAGTTACAGAGCTTGTGGTTCCACAATTCCACCTCCGT | |
| | snIleGluSerTyrArgAlaCysGlySerThrIleProProProT | |
| 451 | ATATCTCTTCACAAGACCACATCTGGATTAGGTTTCATTCGGATG | |
| | yrIleSerSerGlnAspHisIleTrpIleArgPheHisSerAspA | |
| 496 | ACAACATCTCTAGAAAGGGTTTCAGACTGGCATATTTTTCAGGGA | |
| | spAsnIleSerArgLysGlyPheArgLeuAlaTyrPheSerGlyL | |
| 541 | AATCTGAGGAACCAAATTGTGCTTGTGATCAGTTTCGTTGTGGTA | |
| | ysSerGluGluProAsnCysAlaCysAspGlnPheArgCysGlyA | |
| 586 | ATGGAAAGTGTATACCAGAAGCCTGGAAATGTAATAACATGGATG | |
| | snGlyLysCysIleProGluAlaTrpLysCysAsnAsnMetAspG | |
| 631 | AATGTGGAGATAGTTCCGATGAAGAGATCTGTGCCAAAGAAGCAA | |
| | luCysGlyAspSerSerAspGluGluIleCysAlaLysGluAlaA | |
| 676 | ATCCTCCAACTGCTGCTGCTTTTCAACCCTGTGCTTACAACCAGT | |
| | snProProThrAlaAlaAlaPheGlnProCysAlaTyrAsnGlnP | |
| 721 | TCCAGTGTTTATCCCGTTTTACCAAAGTTTACACTTGCCTCCCCG | |
| | heGlnCysLeuSerArgPheThrLysValTyrThrCysLeuProG | |
| 766 | AATCTTTAAAATGTGATGGGAACATTGACTGCCTTGACCTAGGAG | |
| | luSerLeuLysCysAspGlyAsnIleAspCysLeuAspLeuGlyA | |
| 811 | ATGAGATAGACTGTGATGTGCCAACATGTGGGCAATGGCTAAAAT | |
| | spGluIleAspCysAspValProThrCysGlyGlnTrpLeuLysT | |
| 856 | ATTTTTATGGTACTTTTAATTCTCCCAATTATCCAGACTTTTATC | |
| | yrPheTyrGlyThrPheAsnSerProAsnTyrProAspPheTyrP | |
| 901 | CTCCTGGAAGCAATTGCACCTGGTTAATAGACACTGGTGATCACC | |
| | roProGlySerAsnCysThrTrpLeuIleAspThrGlyAspHisA | |
| 946 | GTAAAGTCATTTTACGCTTCACTGACTTTAAACTTGATGGTACTG | |
| | rgLysValIleLeuArgPheThrAspPheLysLeuAspGlyThrG | |
| 991 | GTTATGGTGATTATGTCAAAATATATGATGGATTAGAGGAGAATC | |
| | lyTyrGlyAspTyrValLysIleTyrAspGlyLeuGluGluAsnP | |
| 1036 | CACACAAGCTTTTGCGTGTGTTGACAGCTTTTGATTCTCATGCAC | |
| | roHisLysLeuLeuArgValLeuThrAlaPheAspSerHisAlaP | |
| 1081 | CTCTTACAGTTGTTTCTTCTTCTGGACAGATAAGGGTACATTTTT | |
| | roLeuThrValValSerSerSerGlyGlnIleArgValHisPheC | |
| 1126 | CTGCTGATAAAGTGAATGCTGCAAGGGGATTTAATGCTACTTACC | |
| | ysAlaAspLysValAsnAlaAlaArgGlyPheAsnAlaThrTyrG | |
| 1171 | AAGTAGATGGGTTCTGTTTGCCATGGGAAATACCCTGTGGAGGTA | |
| | lnValAspGlyPheCysLeuProTrpGluIleProCysGlyGlyA | |
| 1216 | ACTGGGGGTGTTATACTGAGCAGCAGCGTCGTGATGGGTATTGGC | |
| | snTrpGlyCysTyrThrGluGlnGlnArgArgAspGlyTyrTrpH | |
| 1261 | ATTGCCCAAATGGAAGGGATGAAACCAATTGTACCATGTGCCAGA | |
| | isCysProAsnGlyArgAspGluThrAsnCysThrMetCysGlnL | |
| 1306 | AGGAAGAATTTCCATGTTCCCGAAATGGTGTCTGCTATCCTCGCT | |
| | ysGluGluPheProCysSerArgAsnGlyValCysTyrProArgS | |
| 1351 | CTGATCGCTGCAACTACCAGAATCATTGCCCAAATGGCAAACAGA | |
| | erAspArgCysAsnTyrGlnAsnHisCysProAsnGlyLysGlnA | |
| 1396 | ACCCATCTACTTGGTAAGTAGCATTAAATCCCCTTGCAGCATTCA | |
| | snProSerThrTrp | |
| 1441 | C | |

A search of the sequence databases using BLASTP reveals that the protein of clone 17941787.0.3 has 398 of 403 residues (98%) identical to, and 400 of 403 residues (99%) positive with the 859 residue human ST7 protein (SPTREMBL-ACC:Q9Y561).

The proteins of the invention encoded by clone 17941787.0.3 include the full protein disclosed as being encoded by the ORFs described herein, as well as any mature protein arising therefrom as a result of posttranslational modifications. Thus, the proteins of the invention encompass both a precursor and any active forms of the 17941787.0.3 protein.

POLY11

A POLY11 nucleic acid according to the invention includes the nucleic acid sequence represented in Clone 20936375.0.104. RNA sequences homologous to this clone are found in the kidney. A representation of the nucleotide sequence of clone 20936375.0.104 is given in Table 12. This clone includes a nucleotide sequence (SEQ ID NO:21) of 2056 bp. This nucleotide sequence has an open reading frame encoding a polypeptide of 534 amino acid residues (represented in Table 12; SEQ ID NO:22) with a predicted molecular weight of 60037.3 Da. The start codon is at nucleotides 7-9 and the stop codon is at nucleotides 1609-1611. The protein of SEQ ID NO:22 is predicted by the PSORT program to localize in the plasma membrane with a certainty of 0.7300 and to have an uncleavable N-terminal signal sequence. The program SignalP predicts that there is no signal peptide.

TABLE 12
Translated Protein - Frame: 1 - Nucleotide 7 to 1608

| | | |
|---|---|---|
| 1 | CGCTCCATGTATNAGTTTCATGCAGGCTCTTGGGAAAGCTGGTGC | (SEQ ID NO:21) |
| | MetTyr---PheHisAlaGlySerTrpGluSerTrpCys | (SEQ ID NO:22) |
| 46 | TGCTGCTGCCTGATTCCCGCCGACAGACCTTGGGACCGGGGCCAA | |
| | CysCysCysLeuIleProAlaAspArgProTrpAspArgGlyGln | |
| 91 | CACTGGCAGCTGGAGATGGCGGACACGAGATCCGTGCACGAGACT | |
| | HisTrpGlnLeuGluMetAlaAspThrArgSerValHisGluThr | |
| 136 | AGGTTTGAGGCGGCCGTGAAGGTGATCCAGAGTTTGCCGAAGAAT | |
| | ArgPheGluAlaAlaValLysValIleGlnSerLeuProLysAsn | |
| 181 | GATTCATTCCAGCCAACAAATGAAATGATGCTTAAATTTTATAGC | |
| | AspSerPheGlnProThrAsnGluMetMetLeuLysPheTyrSer | |
| 226 | TTCTATAAGCAGGCAACTGAAGGACCCTGTAAACTTTCAAGGCCT | |
| | PheTyrLysGlnAlaThrGluGlyProCysLysLeuSerArgPro | |
| 271 | GGATTTTGGGATCCTATTGGAAGATATAAATGGGATGCTTGGAGT | |
| | GlyPheTrpAspProIleGlyArgTyrLysTrpAspAlaTrpSer | |
| 316 | TCACTGGGTGATATGACCAAAGAGGAAGCCATGATTGCATATGTT | |
| | SerLeuGlyAspMetThrLysGluGluAlaMetIleAlaTyrVal | |
| 361 | GAAGAAATGAAAAAGATTATTGAAACTATGCCAATGACTGAGAAA | |
| | GluGluMetLysLysIleIleGluThrMetProMetThrGluLys | |
| 406 | GTTGAAGAATTGCTGCGTGTCATAGGTCCATTTTATGAAATTGTC | |
| | ValGluGluLeuLeuArgValIleGlyProPheTyrGluIleVal | |
| 451 | GAGGACAAAAAGAGTGGCAGGAGTTCTGATATAACCTCAGTCCGA | |
| | GluAspLysLysSerGlyArgSerSerAspIleThrSerValArg | |
| 496 | CTGGAGAAAATCTCTAAATGTTTAGAAGATCTTGGTAATGTTCTC | |
| | LeuGluLysIleSerLysCysLeuGluAspLeuGlyAsnValLeu | |
| 541 | ACTTCTACTCCAAACGCCAAAACCGTTAATGGTAAAGCTGAAAGC | |
| | ThrSerThrProAsnAlaLysThrValAsnGlyLysAlaGluSer | |
| 586 | AGTGACAGTGGAGCCGAGTCTGAGGAAGAAGAGGCCCAAGAAGAA | |
| | SerAspSerGlyAlaGluSerGluGluGluGluAlaGlnGluGlu | |
| 631 | GTGAAAGGAGCAGAACAAAGTGATAATGATAAGAAAATGATGAAG | |
| | ValLysGlyAlaGluGlnSerAspAsnAspLysLysMetMetLys | |
| 676 | AAGTCAGCAGACCATAAGAATTTGGAAGTCATTGTCACTAATGGC | |
| | LysSerAlaAspHisLysAsnLeuGluValIleValThrAsnGly | |
| 721 | TATGATAAAGATGGCTTTGTTCAGGATATACAGAATGACATTCAT | |
| | TyrAspLysAspGlyPheValGlnAspIleGlnAsnAspIleHis | |
| 766 | GCCAGTTCTTCCCTGAATGGCAGAAGCACTGAAGAAGTAAAGCCC | |
| | AlaSerSerSerLeuAsnGlyArgSerThrGluGluValLysPro | |
| 811 | ATTGATGAAAACTTGGGGCAAACTGGAAAATCTGCTGTTTGCATT | |
| | IleAspGluAsnLeuGlyGlnThrGlyLysSerAlaValCysIle | |
| 856 | CACCAAGATATAAATGATGATCATGTTGAAGATGTTACAGGAATT | |
| | HisGlnAspIleAsnAspAspHisValGluAspValThrGlyIle | |
| 901 | CAGCATTTGACAAGCGATTCAGACAGTGAAGTTTACTGTGATTCT | |
| | GlnHisLeuThrSerAspSerAspSerGluValTyrCysAspSer | |
| 946 | ATGGAACAATTTGGACAAGAAGAGTCTTTAGACAGCTTTACGTCC | |
| | MetGluGlnPheGlyGlnGluGluSerLeuAspSerPhethrSer | |
| 991 | AACAATGGACCATTTCAGTATTACTTGGGTGGTCATTCCAGTCAA | |
| | AsnAsnGlyProPheGlnTyrTyrLeuGlyGlyHisSerSerGln | |
| 1036 | CCCATGGAAAATTCTGGATTTCGTGAAGATATTCAAGTACCTCCT | |
| | ProMetGluAsnSerGlyPheArgGluAspIleGlnValProPro | |
| 1081 | GGAAATGGCAACATTGGGAATATGCAGGTGGTTGCAGTTGAAGGA | |
| | GlyAsnGlyAsnIleGlyAsnMetGlnValValAlaValGluGly | |
| 1126 | AAAGGTGAAGTCAAGCATGGAGGAGAAGATGGCAGGAATAACAGC | |
| | LysGlyGluValLysHisGlyGlyGluAspGlyArgAsnAsnSer | |
| 1171 | GGAGCACCACACCGGGAGAAGCGAGGCGGAGAAACTGACGAATTC | |
| | GlyAlaProHisArgGluLysArgGlyGlyGluThrAspGluPhe | |
| 1216 | TCTAATGTTAGAAGAGGAAGAGGACATAGGATGCAACACTTGAGC | |
| | SerAsnValArgArgGlyArgGlyHisArgMetGlnHisLeuSer | |
| 1261 | GAAGGAACCAAGGGCCGGCAGGTGGGAAGTGGAGGTGATGGGGAG | |
| | GluGlyThrLysGlyArgGlnValGlySerGlyGlyAspGlyGlu | |
| 1306 | CGCTGGGGCTCCGACAGAGGGTCCCGAGGCAGCCTCAATGAGCAG | |
| | ArgTrpGlySerAspArgGlySerArgGlySerLeuAsnGluGln | |
| 1351 | ATCGCCCTCGTGCTGATGAGACTGCAGGAGGACATGCAGAATGTC | |
| | IleAlaLeuValLeuMetArgLeuGlnGluAspMetGlnAsnVal | |
| 1396 | CTTCAGAGACTGCAGAAACTGGAAACGCTGACTGCTTTGCAGGCA | |
| | LeuGlnArgLeuGlnLysLeuGluThrLeuThrAlaLeuGlnAla | |
| 1441 | AAATCATCAACATCAACATTGCAGACTGCTCCTCAGCCCACCTCA | |
| | LysSerSerThrSerThrLeuGlnThrAlaProGlnProthrSer | |
| 1486 | CAGAGACCATCTTGGTGGCCCTTCGAGATGTCTCCTGGTGTGCTA | |
| | GlnArgProSerTrpTrpProPheGluMetSerProGlyValLeu | |
| 1531 | ACGTTTGCCATCATATGGCCTTTTATTGCACAGTGGTTGGTGTAT | |
| | ThrPheAlaIleIleTrpProPheIleAlaGlnTrpLeuValTyr | |
| 1576 | TTATACTATCAAAGAAGGAGAAGAAAACTGAACTGAGGAAAATGG | |
| | LeuTyrTyrGlnArgArgArgArgLysLeuAsn | |
| 1621 | TGTTTTCCTCAAGAAGACTACTGGAACTGGATGACCTCAGAATGA | |
| 1666 | ACTGGATTGTGGTGTTCACAAGAAAATCTTAGTTTGTGATGATTA | |
| 1711 | CATTGCTTTTTGTTGTCCAGTAGTTTAGTTTGTGTACATATATAC | |
| 1756 | ACATATATATTTTGCACTACACAAACGATAACATTTTAAGGACTA | |
| 1801 | ATATTGCTGATACTTGAATAATCAATCTCTACTAGGTTATAAGTA | |
| 1846 | GTATACACAGATTTACCCTGCCCTTGAACTTGAAGGACATTAAAT | |
| 1891 | TATTAATGATCATTTGGTAACATGTTTACCTGATTATCTTCCATA | |
| 1936 | GAGTAACATAAGCTGCTTTTCAAAGGTACCATTGTGATAATGAGA | |
| 1981 | TCAAATTTATAAGTTATTATTTTTAATTTTCTAAATTAAATAAAA | |
| 2026 | GAAAGAATGCAAAAAAAAAAAAAAAAAAAAA | |

A search of the sequence databases using BLASTP reveals that the protein of clone 20936375.0.104 has 453 of 534 residues (84%) identical to, and 483 of 534 residues (90%) positive with the 533 residue bovine endozepine-related protein precursor (membrane-associated diazepam binding inhibitor) (MA-DBI) (ACC:P07106). It also has a low similarity over 91 residues to the 359 residue human peroxisomal D3,D2-enoyl-coA isomerase 3 (ACC:AAD34173).

The proteins of the invention encoded by clone 20936375.0.104 include the full protein disclosed as being encoded by the ORFs described herein, as well as any mature protein arising therefrom as a result of posttranslational modifications. Thus, the proteins of the invention encompass both a precursor and any active forms of the 20936375.0.104 protein.

POLY12

A POLY12 nucleic acid according to the invention includes the nucleic acid sequence represented in the nucleic acid sequence represented in Clone 21636818.0.57. RNA sequences homologous to this clone are found in the uterus and bone marrow. A representation of the nucleotide sequence of clone 21636818.0.57 is given in Table 13. This clone includes a nucleotide sequence (SEQ ID NO:23) of 1544 bp. This nucleotide sequence has an open reading frame encoding a polypeptide of 151 amino acid residues (represented in Table 13; SEQ ID NO:24) with a predicted molecular weight of 16776.3 Da. The start codon is at nucleotides 274-276 and the stop codon is at nucleotides 727-729. The protein of SEQ ID NO:24 is predicted by the PSORT program to localize extracellularly with a certainty of 0.3700 and to have a cleavable N-terminal signal sequence. The program SignalP predicts that the most likely cleavage site occurs between residues 19 and 20: SNP-SH.

TABLE 13
Translated Protein - Frame: 1 - Nucleotide 274 to 726

| | | |
|---|---|---|
| 1 | ATAACCAACAATGGCAATGACCATCCACCACAGGCCCGGCACTCA | (SEQ ID NO:23) |
| 46 | TGATCACAATGTGAGGTAGACACTGCTGCCCCCACTTCACAGACA | |
| 91 | AGGAAATCGAAGTTCAGAGCTATTCAATCACCTCTTCGAGGCCGC | |
| 136 | ACAATTCACCAGCAGCAAAGCTTGGCCTGGGACCCAGTTGCACCT | |
| 181 | GGCCCAAAGTCTGAGGTCTCCTCCACTGCCTCCTCCCTTCTTCAT | |
| 226 | CCCCAGCATCAGGCCCAGATCTGGGCCTCCACCACCTTCAGCCTG | |
| 271 | ACTATGGCGAGGTCTTTCTACCTGGTCTCCCTGCCTCTGGTACTT | |
| | MetAlaArgSerPheTyrLeuValSerLeuProLeuValLeu | (SEQ ID NO:24) |
| 316 | CCCTCCTCCAACCCCTCTCACGTGTGGCTGACCAGGTGTACTCAT | |
| | ProSerSerAsnProSerHisValTrpLeuThrArgCysThrHis | |
| 361 | GTCATTCTCTTTCAAAAAAGTATTCAGGGCCTTCAATACATACAA | |
| | ValIleLeuPheGlnLysSerIleGlnGlyLeuGlnTyrIleGln | |
| 406 | AATCTGGAGTGGTCCTCACCTGTCACTGAATCCTGGCTATGTTGC | |
| | AsnLeuGluTrpSerSerProValThrGluSerTrpLeuCysCys | |
| 451 | AGGACCCAGCCAAAGACTTTTTCCACAAAGTCTTCTCCCGAAACC | |
| | ArgThrGlnProLysThrPheSerThrLysSerSerProGluThr | |
| 496 | TTAGCTCTCACACTCTCTCCCTCTCTCCCCTCTGCCCCGCGTCTG | |
| | LeuAlaLeuThrLeuSerProSerLeuProSerAlaProArgLeu | |
| 541 | TACCTGGTTTCTCTCTGTGCTCTCGTAACACCTCAGGCCAAGGTA | |
| | TyrLeuValSerLeuCysAlaLeuValThrProGlnAlaLysVal | |
| 586 | ATTCCGTGTGGTGGAGGCCTGTCTCGTGCACTGCGGGATGTTCAG | |
| | IleProCysGlyGlyGlyLeuSerArgAlaLeuArgAspValGln | |
| 631 | CAGCATCCCTGGCTCCTGCCCACCCCCAATCATGACCATCAAAAA | |
| | GlnHisProTrpLeuLeuProThrProAsnHisAspHisGlnLys | |
| 676 | TGTCCCCAGACAACTACCAAGTATCCCCTGGAGGGCAAAATCACC | |
| | CysProGlnthrThrThrLysTyrProLeuGluGlyLysIleThr | |
| 721 | CTGGGTTGAGAGCCACTGTCTCAGACAGTACTTCGGCCACTGCCT | |
| | LeuGly | |
| 766 | CGGACCGCACCTTGTGGCTCAGGGAATGAGTATAGACTTCTCTGA | |
| 811 | AGGGGCTGCCTGCATTTTAAAAAGCCACCACCAGAACTCACCATT | |
| 856 | GCGAGTCAATTTGGGGGTGGTTCGGGAATTCACCAGGCTCTCCAT | |
| 901 | TTCCAGGTGAATATCTTTTGTTTCGCCAGTATCATATAAATGGCG | |
| 946 | CACCTGGCAGAAATTTACAAAGTCAAACAGAAAGAAAATCAAATA | |
| 991 | TCAAGTAACAAATTCCTACCTAAGAGCAGAATGGCTGAAAGGAAA | |
| 1036 | GTCTGGGATAGAAGGCTGGGTAAAGTTCAGACAGAAGTTAGGATG | |
| 1081 | ATGACCTTTAAATCTTTTATGAAATAAGGTGGGGTTTGAGAAAAA | |
| 1126 | CAAACACATTTTAACTGTGGGACTTTGTGCTAGAAATCAGCTACA | |
| 1171 | CAGGGCCGGGTGCGGTGGCTCACACCTATAATCCCTATAGTTGGG | |
| 1216 | GAGGCCGAGGCCAGCAGATGGCTTAAGGCTAGGAGTTTGAGACCA | |
| 1261 | GTTTGGGAAACACAGGGAGGCCTTGTCTCTACAAAAAATTTAAAA | |
| 1306 | ATTAGCTGTAGTCCCAGCTACTTGGGAGGCTGAAGTGGGAGGATC | |
| 1351 | CCTTGAGACCAGGAAGTCAAAGCTTCAGTGAGCTGAGATAGTGCC | |
| 1396 | CCCACACCCCAAAAAGCCTGGGCAACAAAGCAAGACTTTGTTTCN | |
| 1441 | AAAAAAAAAAAAAGAAAAAAGAAAAGAGAGAAAAAATTAGTTACT | |
| 1486 | CGGTCATGGAGGTCCCTCTAAGAGTCTCGGTTGGACTACTAATAA | |
| 1531 | TGCTGCCTCTTAAC | |

A search of the sequence databases using BLASTP reveals that the protein of clone 21636818.0.57 has 17 of 49 residues (34%) identical to, and 26 of 49 residues (53%) positive with the 386 residue PKM101 conjugation proteins (TRAL), (TRAM), (TRAA), (TRAB), (TRAC), (TRAB), (TRAC), (TRAD), (TRAN), (TRAE), (TRAO), (TRAF), (TRAG), entry exclusion protein (EEX), (KIKA), (KORB), (KORA) and endonuclease (NUC) genes, complete CDS (TRAM)(TRAB) (TRAB) (TRAD) (TRAE) (TRAF) (EEX) (KORB) (KORA) (NUC) from Escherichia coli, (ACC: SPTREMBL Q46705).

The proteins of the invention encoded by clone 21636818.0.57include the full protein disclosed as being encoded by the ORFs described herein, as well as any mature protein arising therefrom as a result of posttranslational modifications. Thus, the proteins of the invention encompass both a precursor and any active forms of the 21636818.0.57 protein.

POLY13

A POLY13 nucleic acid according to the invention includes the nucleic acid sequence represented in the nucleic acid sequence represented in Clone 20468752-0-18_update. RNA sequences homologous to this clone are found in the placenta. A representation of the nucleotide sequence of clone 20468752-0-18_update is given in Table 14. This clone includes a nucleotide sequence (SEQ ID NO:25) of 2306 bp. This nucleotide sequence has an open reading frame encoding a polypeptide of 720 amino acid residues (represented in Table 14; SEQ ID NO:26) with a predicted molecular weight of 80141.8 Da. The start codon is at nucleotides 128-130 and the stop codon is at nucleotides 2287-2289. The protein of SEQ ID NO:26 is predicted by the PSORT program to localize extracellularly with a certainty of 0.3700 and to have a cleavable N-terminal signal sequence. The program SignalP predicts that the most likely cleavage site occurs between residues 21 and 22: ISS-LP.

TABLE 14
Translated Protein - Frame: 2 - Nucleotide 128 to 2287

| | | |
|---|---|---|
| 1 | GAGCTGAAACCCGAGCTCCCGCTCAGCTGGGGCTCGGGGAGGTCC | (SEQ ID NO:25) |
| 46 | CTGTAAAACCCGCCTGCCCCCGGCCTCCCTGGGTCCCTCCTCTCC | |
| 91 | CTCCCCAGTAGACGCTCGGACACCAGCCGCGGCAAGGATGGAGCT | |
| | MetGluLe | (SEQ ID NO:26) |
| 136 | GGGTTGCTGGACGCAGTTGGGGCTCACTTTTCTTCAGCTCCTTCT | |
| | uGlyCysTrpThrGlnLeuGlyLeuThrPheLeuGlnLeuLeuLe | |
| 181 | CATCTCGTCCTTGCCAAGAGAGTACACAGTCATTAATGAAGCCTG | |
| | uIleSerSerLeuProArgGluTyrThrValIleAsnGluAlaCy | |
| 226 | CCCTGGAGCAGAGTGGAATATCATGTGTCGGGAGTGCTGTGAATA | |
| | sProGlyAlaGluTrpAsnIleMetCysArgGluCysCysGluTy | |
| 271 | TGATCAGATTGAGTGCGTCTGCCCCGGAAAGAGGGAAGTCGTGGG | |
| | rAspGlnIleGluCysValCysProGlyLysArgGluValValGl | |
| 316 | TTATACCATCCCTTGCTGCAGGAATGAGGAGAATGAGTGTGACTC | |
| | yTyrThrIleProCysCysArgAsnGluGluAsnGluCysAspSe | |
| 361 | CTGCCTGATCCACCCAGGTTGTACCATCTTTGAAAACTGCAAGAG | |
| | rCysLeuIleHisProGlyCysThrIlePheGluAsnCysLysSe | |
| 406 | CTGCCGAAATGGCTCATGGGGGGGTACCTTGGATGACTTCTATGT | |
| | rCysArgAsnGlySerTrpGlyGlyThrLeuAspAspPheTyrVa | |
| 451 | GAAGGGGTTCTACTGTGCAGAGTGCCGAGCAGGTGGTACGGAGG | |
| | lLysGlyPheTyrCysAlaGluCysArgAlaGlyTrpTyrGlyGl | |
| 496 | AGACTGCATGCGATGTGGCCAGGTTCTGCGAGCCCCAAAGGGTCA | |
| | yAspCysMetArgCysGlyGlnValLeuArgAlaProLysGlyGl | |
| 541 | GATTTTGTTGGAAAGCTATCCCCTAAATGCTCACTGTGAATGGAC | |
| | nIleLeuLeuGluSerTyrProLeuAsnAlaHisCysGluTrpTh | |
| 586 | CATTCATGCTAAACCTGGGTTTGTCATCCAACTAAGATTTGTCAT | |
| | rIleHisAlaLysProGlyPheValIleGlnLeuArgPheValMe | |
| 631 | GTTGAGCCTGGAGTTTGACTACATGTGCCAGTATGACTATGTTGA | |
| | tLeuSerLeuGluPheAspTyrMetCysGlnTyrAspTyrValGl | |
| 676 | GGTTCGTGATGGAGACAACCGCGATGGCCAGATCATCAAGCGTGT | |
| | uValArgAspGlyAspAsnArgAspGlyGlnIleIleLysArgVa | |
| 721 | CTGTGGCAACGAGCGGCCAGCTCCTATCCAGAGCATAGGATCCTC | |
| | lCysGlyAsnGluArgProAlaProIleGlnSerIleGlySerSe | |
| 766 | ACTCCACGTCCTCTTCCACTCCGATGGCTCCAAGAATTTTGACGG | |
| | rLeuHisValLeuPheHisSerAspGlySerLysAsnPheAspGl | |
| 811 | TTTCCATGCCATTTATGAGGAGATCACAGCATGCTCCTCATCCCC | |
| | yPheHisAlaIleTyrGluGluIleThrAlaCysSerSerSerPr | |
| 856 | TTGTTTCCATGACGGCACGTGCGTCCTTGACAAGGCTGGATCTTA | |
| | oCysPheHisAspGlyThrCysValLeuAspLysAlaGlySerTy | |
| 901 | CAAGTGTGCCTGCTTGGCAGGCTATACTGGGCAGCGCTGTGAAAA | |
| | rLysCysAlaCysLeuAlaGlyTyrThrGlyGlnArgCysGluAs | |
| 946 | TCTCCTTGAAGAAAGAAACTGCTCAGACCCTGGGGGCCCAGTCAA | |
| | nLeuLeuGluGluArgAsnCysSerAspProGlyGlyProValAs | |
| 991 | TGGGTACCAGAAAATAACAGGGGGCCCTGGGCTTATCAACGGACG | |
| | nGlyTyrGlnLysIleThrGlyGlyProGlyLeuIleAsnGlyAr | |
| 1036 | CCATGCTAAAATTGGCACCGTGGTGTCTTTCTTTTGTAACAACTC | |
| | gHisAlaLysIleGlyThrValValSerPhePheCysAsnAsnSe | |
| 1081 | CTATGTTCTTAGTGGCAATGAGAAAAGAACTTGCCAGCAGAATGG | |
| | rTyrValLeuSerGlyAsnGluLysArgThrCysGlnGlnAsnGl | |
| 1126 | AGAGTGGTCAGGGAAACAGCCCATCTGCATAAAAGCCTGCCGAGA | |
| | yGluTrpSerGlyLysGlnProIleCysIleLysAlaCysArgGl | |
| 1171 | ACCAAAGATTTCAGACCTGGTGAGAAGGAGAGTTCTTCCGATGCA | |
| | uProLysIleSerAspLeuValArgArgArgValLeuProMetGl | |
| 1216 | GGTTCAGTCAAGGGAGACACCATTACACCAGCTATACTCAGCGGC | |
| | nValGlnSerArgGluThrProLeuHisGlnLeuTyrSerAlaAl | |
| 1261 | CTTCAGCAAGCAGAAACTGCAGAGTGCCCCTACCAAGAAGCCAGC | |
| | aPheSerLysGlnLysLeuGlnSerAlaProThrLysLysProAl | |
| 1306 | CCTTCCCTTTGGAGATCTGCCCATGGGATACCAACATCTGCATAC | |
| | aLeuProPheGlyAspLeuProMetGlyTyrGlnHisLeuHisTh | |
| 1351 | CCAGCTCCAGTATGAGTGCATCTCACCCTTCTACCGCCGCCTGGG | |
| | rGlnLeuGlnTyrGluCysIleSerProPheTyrArgArgLeuGl | |
| 1396 | CAGCAGCAGGAAGACATGTCTGAAGACTGGGAAGTGGAGTGGGCG | |
| | ySerSerArgLysThrCysLeuLysThrGlyLysTrpSerGlyAr | |
| 1441 | GGCACCATCCTGCATCCCTATCTGCGGGAAAATTGAGAACATCAC | |
| | gAlaProSerCysIleProIleCysGlyLysIleGluAsnIleTh | |
| 1486 | TGCTCCAAAGACCCAAGGGTTGCGCTGGCCGTGGCAGGCAGCCAT | |
| | rAlaProLysThrGlnGlyLeuArgTrpProTrpGlnAlaAlaIl | |
| 1531 | CTACAGGAGGACCAGCGGGGTGCATGACGGCAGCCTACACAAGGG | |
| | eTyrArgArgThrSerGlyValHisAspGlySerLeuHisLysGl | |
| 1576 | AGCGTGGTTCCTAGTCTGCAGCGGTGCCCTGGTGAATGAGCGCAC | |
| | yAlaTrpPheLeuValCysSerGlyAlaLeuValAsnGluArgTh | |
| 1621 | TGTGGTGGTGGCTGCCCACTGTGTTACTGACCTGGGGAAGGTCAC | |
| | rValValValAlaAlaHisCysValThrAspLeuGlyLysValTh | |
| 1666 | CATGATCAAGACAGCAGACCTGAAAGTTGTTTTGGGGAAATTCTA | |
| | rMetIleLysThrAlaAspLeuLysValValLeuGlyLysPheTy | |
| 1711 | CCGGGATGATGACCGGGATGAGAAGACCATCCAGAGCCTACAGAT | |
| | rArgAspAspAspArgAspGluLysThrIleGlnSerLeuGlnIl | |
| 1756 | TTCTGCTATCATTCTGCATCCCAACTATGACCCCATCCTGCTTGA | |
| | eSerAlaIleIleLeuHisProAsnTyrAspProIleLeuLeuAs | |
| 1801 | TGCTGACATCGCCATCCTGAAGCTCCTAGACAAGGCCCGTATCAG | |
| | pAlaAspIleAlaIleLeuLysLeuLeuAspLysAlaArgIleSe | |
| 1846 | CACCCGAGTCCAGCCCATCTGCCTCGCTGCCAGTCGGGATCTCAG | |
| | rThrArgValGlnProIleCysLeuAlaAlaSerArgAspLeuSe | |
| 1891 | CACTTCCTTCCAGGAGTCCCACATCACTGTGGCTGGCTGGAATGT | |
| | rThrSerPheGlnGluSerHisIleThrValAlaGlyTrpAsnVa | |
| 1936 | CCTGGCAGACGTGAGGAGCCCTGGCTTCAAGAACGACACACTGCG | |
| | lLeuAlaAspValArgSerProGlyPheLysAsnAspthrLeuAr | |
| 1981 | CTCTGGGGTGGTCAGTGTGGTGGACTCGCTGCTGTGTGAGGAGCA | |
| | gSerGlyValValSerValValAspSerLeuLeuCysGluGluGl | |
| 2026 | GCATGAGGACCATGGCATCCCAGTGAGTGTCACTGATAACATGTT | |
| | nHisGluAspHisGlyIleProValSerValthrAspAsnMetPh | |
| 2071 | CTGTGCCAGCTGGGAACCCACTGCCCCTTCTGATATCTGCACTGC | |
| | eCysAlaSerTrpGluProThrAlaProSerAspIleCysThrAl | |
| 2116 | AGAGACAGGAGGCATCGCGGCTGTGTCCTTCCCGGGACGAGCATC | |
| | aGluThrGlyGlyIleAlaAlaValSerPheProGlyArgAlaSe | |
| 2161 | TCCTGAGCCACGCTGGCATCTGATGGGACTGGTCAGCTGGAGCTA | |
| | rProGluProArgTrpHisLeuMetGlyLeuValSerTrpSerTy | |
| 2206 | TGATAAAACATGCAGCCACAGGCTCTCCACTGCCTTCACCAAGGT | |
| | rAspLysThrCysSerHisArgLeuSerThrAlaPhethrLysVa | |
| 2251 | GCTGCCTTTTAAAGACTGGATTGAAAGAAATATGAAATGAACCAT | |
| | lLeuProPheLysAspTrpIleGluArgAsnMetLys | |
| 2296 | GCTCATGCACT | |

A search of the sequence databases using BLASTP reveals that the protein of clone 20468752-0-18_update has 157 of 454 residues (34%) identical to, and 234 of 454 residues (51%) positive with, the 1019 residue factor C protein from Carcinoscorpius rotundicauda (SOUTHEAST ASIAN HORSESHOE CRAB) (SPTREMBL-ACC:Q26422). In addition, the 720 residue protein of clone 20468752-0-18_updat has 180 of 181 residues (99%) identical to, and 181 of 181 residues (100%) positive with the 181 residue fragment of a human hypothetical 20.0 kDa protein (TREMBLNEW-ACC:CAB43317), starting at residue 540.

The proteins of the invention encoded by clone 20468752-0-18_update include the full protein disclosed as being encoded by the ORFs described herein, as well as any mature protein arising therefrom as a result of posttranslational modifications. Thus, the proteins of the invention encompass both a precursor and any active forms of the 21636818.0.57 protein.

POLYX Nucleic Acids

The novel nucleic acids of the invention include those that encode a POLYX or POLYX-like protein, or biologically-active portions thereof. The nucleic acids include nucleic acids encoding polypeptides that include the amino acid sequence of one or more of SEQ ID NO: 2, 4, 6, 8, 10, 12, 14, 16, 18, 20, 22, 24, and 26. The encoded polypeptides can thus include, e.g., the amino acid sequences of SEQ ID NO:2, 4, 6, 8, 10, 12, 14, 16, 18, 20, 22, 24, and 26.

In some embodiments, a nucleic acid encoding a polypeptide having the amino acid sequence of one or more of SEQ ID NO: 2, 4, 6, 8, 10, 12, 14, 16, 18, 20, 22, 24, and 26 includes the nucleic acid sequence of any of SEQ ID NO: 1, 3, 5, 7, 9, 11, 13, 15, 17, 19, 21, 23, and 25, or a fragment thereof. Additionally, the invention includes mutant or variant nucleic acids of any of SEQ ID NO: 1, 3, 5, 7, 9, 11, 13, 15, 17, 19, 21, 23, and 25, or a fragment thereof, any of whose bases may be changed from the disclosed sequence while still encoding a protein that maintains its POLYX-like biological activities and physiological functions. The invention further includes the complement of the nucleic acid sequence of any of SEQ ID NO: 1, 3, 5, 7, 9, 11, 13, 15, 17, 19, 21, 23, and 25, including fragments, derivatives, analogs and homologs thereof. The invention additionally includes nucleic acids or nucleic acid fragments, or complements thereto, whose structures include chemical modifications.

Also included are nucleic acid fragments sufficient for use as hybridization probes to identify POLYX-encoding nucleic acids (e.g., POLYX mRNA) and fragments for use as polymerase chain reaction (PCR) primers for the amplification or mutation of POLYX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments, and homologs thereof. The nucleic acid molecule can be single-stranded or double-stranded, but preferably is double-stranded DNA.

As utilized herein, the term “probes” refer to nucleic acid sequences of variable length, preferably between at least about 10 nucleotides (nt), 100 nt, or as many as about, e.g., 6,000 nt, depending upon the specific use. Probes are used in the detection of identical, similar, or complementary nucleic acid sequences. Longer length probes are usually obtained from a natural or recombinant source, are highly specific and much slower to hybridize than oligomers. Probes may be single- or double-stranded, and may also be designed to have specificity in PCR, membrane-based hybridization technologies, or ELISA-like technologies.

As utilized herein, the term “isolated” nucleic acid molecule is a nucleic acid that is separated from other nucleic acid molecules that are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated POLYX nucleic acid molecule can contain less than approximately 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full length gene product, encoded by the corresponding gene. Alternatively, it may be defined as the polypeptide, precursor or proprotein encoded by an open reading frame described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

A nucleic acid molecule of the invention, e.g., a nucleic acid molecule having the nucleotide sequence of SEQ ID NO: 1, 3, 5, 7, 9, 11, 13, 15, 17, 19, 21, 23, and 25, or a complement of any of these nucleotide sequences, can be isolated using standard molecular biology techniques and the sequence information provided herein. Using all or a portion of the nucleic acid sequence of any of SEQ ID NO: 1, 3, 5, 7, 9, 11, 13, 15, 17, 19, 21, 23, and 25 as a hybridization probe, POLYX nucleic acid sequences can be isolated using standard hybridization and cloning techniques (e.g., as described in Sambrook et al., eds., Molecular Cloning: A Laboratory Manual 2^{nd} Ed., Cold Spring Harbor Laboratory Press, Cold Spring Harbor, N.Y., 1989; and Ausubel, et al., eds., Current Protocols in Molecular Biology, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to POLYX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment, an oligonucleotide comprising a nucleic acid molecule less than 100 nt in length would further comprise at lease 6 contiguous nucleotides of any of SEQ ID NO: 1, 3, 5, 7, 9, 11, 13, 15, 17, 19, 21, 23, and 25, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in any of SEQ ID NO: 1, 3, 5, 7, 9, 11, 13, 15, 17, 19, 21, 23, and 25, or a portion of this nucleotide sequence. A nucleic acid molecule that is complementary to the nucleotide sequence shown in any of SEQ ID NO: 1, 3, 5, 7, 9, 11, 13, 15, 17, 19, 21, 23, and 25 is one that is sufficiently complementary to the nucleotide sequence shown that it can hydrogen bond with little or no mismatches to the nucleotide sequence shown in of any of SEQ ID NO: 1, 3, 5, 7, 9, 11, 13, 15, 17, 19, 21, 23, and 25, thereby forming a stable duplex.

As used herein, the term “complementary” refers to Watson-Crick or Hoogsteen base-pairing between nucleotides units of a nucleic acid molecule, whereas the term “binding” is defined as the physical or chemical interaction between two polypeptides or compounds or associated polypeptides or compounds or combinations thereof. Binding includes ionic, non-ionic, Von der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Additionally, the nucleic acid molecule of the invention can comprise only a portion of the nucleic acid sequence of any of SEQ ID NO: 1, 3, 5, 7, 9, 11, 13, 15, 17, 19, 21, 23, and 25, e.g., a fragment that can be used as a probe or primer, or a fragment encoding a biologically active portion of POLYX. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild-type.

Derivatives and analogs may be full-length or other than full-length, if the derivative or analog contains a modified nucleic acid or amino acid, as described infra. Derivatives or analogs of the nucleic acids or proteins of the invention include, but are not limited to, molecules comprising regions that are substantially homologous to the nucleic acids or proteins of the invention, in various embodiments, by at least about 70%, 80%, 85%, 90%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et at., Current Protocols in Molecular Biology, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489), which is incorporated herein by reference in its entirety.

As utilized herein, the term “homologous nucleic acid sequence” or “homologous amino acid sequence,” or variations thereof, refer to sequences characterized by a homology at the nucleotide level or amino acid level as discussed supra. Homologous nucleotide sequences encode those sequences coding for isoforms of POLYX polypeptide. Isoforms can be expressed in different tissues of the same organism as a result of, e.g., alternative splicing of RNA. Alternatively, isoforms can be encoded by different genes. In the invention, homologous nucleotide sequences include nucleotide sequences encoding for a POLYX polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding human POLYX protein. Homologous nucleic acid sequences include those nucleic acid sequences that encode conservative amino acid substitutions (see below) in any of SEQ ID NO: 2, 4, 6, 8, 10, 12, 14, 16, 18, 20, 22, 24, and 26 as well as a polypeptide having POLYX activity. Biological activities of the POLYX proteins are described below. A homologous amino acid sequence does not encode the amino acid sequence of a human POLYX polypeptide.

The nucleotide sequence determined from the cloning of the human POLYX gene allows for the generation of probes and primers designed for use in identifying the cell types disclosed and/or cloning POLYX homologues in other cell types, e.g., from other tissues, as well as POLYX homologues from other mammals. The probe/primer typically comprises a substantially-purified oligonucleotide. The oligonucleotide typically comprises a region of nucleotide sequence that hybridizes under stringent conditions to at least about 12, 25, 50, 100, 150, 200, 250, 300, 350 or 400 or more consecutive sense strand nucleotide sequence of SEQ ID NO: 1, 3, 5, 7, 9, 11, 13, 15, 17, 19, 21, 23, and 25; or an anti-sense strand nucleotide sequence of SEQ ID NO: 1, 3, 5, 7, 9, 11, 13, 15, 17, 19, 21, 23, and 25; or of a naturally occurring mutant of SEQ ID NO: 1, 3, 5, 7, 9, 11, 13, 15, 17, 19, 21, 23, and 25.

Probes based upon the human POLYX nucleotide sequence can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which mis-express a POLYX protein, such as by measuring a level of a POLYX-encoding nucleic acid in a sample of cells from a subject e.g., detecting POLYX mRNA levels or determining whether a genomic POLYX gene has been mutated or deleted.

As utilized herein, the term “a polypeptide having a biologically-active portion of POLYX refers to polypeptides exhibiting activity similar, but not necessarily identical to, an activity of a polypeptide of the invention, including mature forms, as measured in a particular biological assay, with or without dose dependency. A nucleic acid fragment encoding a biologically-active portion of POLYX can be prepared by isolating a portion of SEQ ID NO: 1, 3, 5, 7, 9, 11, 13, 15, 17, 19, 21, 23, and 25, that encodes a polypeptide having a POLYX biological activity, expressing the encoded portion of POLYX protein (e.g., by recombinant expression in vitro), and assessing the activity of the encoded portion of POLY.

POLYX Variants

The invention further encompasses nucleic acid molecules that differ from the disclosed POLYX nucleotide sequences due to degeneracy of the genetic code. These nucleic acids therefore encode the same POLYX protein as those encoded by the nucleotide sequence shown in SEQ ID NO: 1, 3, 5, 7, 9, 11, 13, 15, 17, 19, 21, 23, and 25. In another embodiment, an isolated nucleic acid molecule of the invention has a nucleotide sequence encoding a protein having an amino acid sequence shown in any of SEQ ID NO: 2, 4, 6, 8, 10, 12, 14, 16, 18, 20, 22, 24, and 26.

In addition to the human POLYX nucleotide sequence shown in any of SEQ ID NO: 1, 3, 5, 7, 9, 11, 13, 15, 17, 19, 21, 23, and 25, it will be appreciated by those skilled in the art that DNA sequence polymorphisms that lead to changes in the amino acid sequences of POLYX may exist within a population (e.g., the human population). Such genetic polymorphism in the POLYX gene may exist among individuals within a population due to natural allelic variation. As used herein, the terms “gene” and “recombinant gene” refer to nucleic acid molecules comprising an open reading frame encoding a POLYX protein, preferably a mammalian POLYX protein. Such natural allelic variations can typically result in 1-5% variance in the nucleotide sequence of the POLYX gene. Any and all such nucleotide variations and resulting amino acid polymorphisms in POLYX that are the result of natural allelic variation and that do not alter the functional activity of POLYX are intended to be within the scope of the invention.

Additionally, nucleic acid molecules encoding POLYX proteins from other species, and thus that have a nucleotide sequence that differs from the human sequence of any of SEQ ID NO: 1, 3, 5, 7, 9, 11, 13, 15, 17, 19, 21, 23, and 25, are intended to be within the scope of the invention. Nucleic acid molecules corresponding to natural allelic variants and homologues of the POLYX cDNAs of the invention can be isolated based on their homology to the human POLYX nucleic acids disclosed herein using the human cDNAs, or a portion thereof, as a hybridization probe according to standard hybridization techniques under stringent hybridization conditions.

In another embodiment, an isolated nucleic acid molecule of the invention is at least 6 nucleotides in length and hybridizes under stringent conditions to the nucleic acid molecule comprising the nucleotide sequence of any of SEQ ID NO: 1, 3, 5, 7, 9, 11, 13, 15, 17, 19, 21, 23, and 25. In another embodiment, the nucleic acid is at least 10, 25, 50, 100, 250, 500 or 750 nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding POLYX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tₘ) for the specific sequence at a defined ionic strength and pH. The Tₘ is the temperature (under defined ionic strength, pH and nucleic acid concentration) at which 50% of the probes complementary to the target sequence hybridize to the target sequence at equilibrium. Since the target sequences are generally present at excess, at Tₘ, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Current Protocols in Molecular Biology, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. An isolated nucleic acid molecule of the invention that hybridizes under stringent conditions to the sequence of any of SEQ ID NO: 1, 3, 5, 7, 9, 11, 13, 15, 17, 19, 21, 23, and 25 corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of any of SEQ ID NO: 1, 3, 5, 7, 9, 11, 13, 15, 17, 19, 21, 23, and 25, or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C. , followed by one or more washes in 1×SSC, 0.1% SDS at 37° C. Other conditions of moderate stringency that may be used are well known in the art. See, e.g., Ausubel et al. (eds.), 1993, Current Protocols in Molecular Biology, John Wiley & Sons, NY, and Kriegler, 1990. Gene Transfer and Expression, A Laboratory Manual, Stockton Press, NY.

In a third embodiment, a nucleic acid that is hybridizable to the nucleic acid molecule comprising the nucleotide sequence of any of SEQ ID NO: 1, 3, 5, 7, 9, 11, 13, 15, 17, 19, 21, 23, and 25, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C. , followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al., (eds.), 1993. Current Protocols in Molecular Biology, John Wiley & Sons, NY, and Kriegler, 1990. Gene Transfer and Expression, A Laboratory Manual, Stockton Press, NY; Shilo and Weinberg, 1981. Proc. Natl. Acad. Sci. USA 78: 6789-6792.

Conservative Mutations

In addition to naturally-occurring allelic variants of the POLYX sequence that may exist in the population, the skilled artisan will further appreciate that changes can be introduced by mutation into the nucleotide sequence of any of SEQ ID NO: 1, 3, 5, 7, 9, 11, 13, 15, 17, 19, 21, 23, and 25, thereby leading to changes in the amino acid sequence of the encoded POLYX protein, without altering the functional ability of the POLYX protein. For example, nucleotide substitutions leading to amino acid substitutions at “non-essential” amino acid residues can be made in the sequence of any of SEQ ID NO: 1, 3, 5, 7, 9, 11, 13, 15, 17, 19, 21, 23, and 25. A “non-essential” amino acid residue is a residue that can be altered from the wild-type sequence of POLYX without altering the biological activity, whereas an “essential” amino acid residue is required for biological activity. For example, amino acid residues that are conserved among the POLYX proteins of the invention, are predicted to be particularly non-amenable to such alteration.

Amino acid residues that are conserved among members of a POLYX family are predicted to be less amenable to alteration. For example, a POLYX protein according to the invention can contain at least one domain that is a typically conserved region in a POLYX family member. As such, these conserved domains are not likely to be amenable to mutation. Other amino acid residues, however, (e.g., those that are not conserved or only semi-conserved among members of the POLYX family) may not be as essential for activity and thus are more likely to be amenable to alteration.

Another aspect of the invention pertains to nucleic acid molecules encoding POLYX proteins that contain changes in amino acid residues that are not essential for activity. Such POLYX proteins differ in amino acid sequence from any of any of SEQ ID NO: 2, 4, 6, 8, 10, 12, 14, 16, 18, 20, 22, 24, and 26, yet retain biological activity. In one embodiment, the isolated nucleic acid molecule comprises a nucleotide sequence encoding a protein, wherein the protein comprises an amino acid sequence at least about 75% homologous to the amino acid sequence of any of SEQ ID NO: 2, 4, 6, 8, 10, 12, 14, 16, 18, 20, 22, 24, and 26. Preferably, the protein encoded by the nucleic acid is at least about 80% homologous to any of SEQ ID NO: 2, 4, 6, 8, 10, 12, 14, 16, 18, 20, 22, 24, and 26, more preferably at least about 90%, 95%, 98%, and most preferably at least about 99% homologous to SEQ ID NO: 2, 4, 6, 8, 10, 12, 14, 16, 18, 20, 22, 24, and 26.

An isolated nucleic acid molecule encoding a POLYX protein homologous to the protein of any of SEQ ID NO: 2, 4, 6, 8, 10, 12, 14, 16, 18, 20, 22, 24, and 26 can be created by introducing one or more nucleotide substitutions, additions or deletions into the corresponding nucleotide sequence (i.e., SEQ ID NO: 1, 3, 5, 7, 9, 11, 13, 15, 17, 19, 21, 23, and 25), such that one or more amino acid substitutions, additions or deletions are introduced into the encoded protein.

Mutations can be introduced into SEQ ID NO: 1, 3, 5, 7, 9, 11, 13, 15, 17, 19, 21, 23, and 25 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Thus, a predicted nonessential amino acid residue in POLYX is replaced with another amino acid residue from the same side chain family. Alternatively, in another embodiment, mutations can be introduced randomly along all or part of a POLYX coding sequence, such as by saturation mutagenesis, and the resultant mutants can be screened for POLYX biological activity to identify mutants that retain activity. Following mutagenesis of SEQ ID NO: 1, 3, 5, 7, 9, 11, 13, 15, 17, 19, 21, 23, and 25, the encoded protein can be expressed by any recombinant technology known in the art and the activity of the protein can be determined.

In one embodiment, a mutant POLYX protein can be assayed for: (i) the ability to form protein:protein interactions with other POLYX proteins, other cell-surface proteins, or biologically-active portions thereof; (ii) complex formation between a mutant POLYX protein and a POLYX receptor; (iii) the ability of a mutant POLYX protein to bind to an intracellular target protein or biologically active portion thereof; (e.g., avidin proteins); (iv) the ability to bind BRA protein; or (v) the ability to specifically bind an anti-POLYX protein antibody.

Antisense Nucleic Acids

Another aspect of the invention pertains to isolated antisense nucleic acid molecules that are hybridizable to or complementary to the nucleic acid molecule comprising the nucleotide sequence of SEQ ID NO: 1, 3, 5, 7, 9, 11, 13, 15, 17, 19, 21, 23, and 25, or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire POLYX coding strand, or to only a portion thereof. Nucleic acid molecules encoding fragments, homologs, derivatives and analogs of a POLYX protein of any of SEQ ID NO: 2, 4, 6, 8, 10, 12, 14, 16, 18, 20, 22, 24, and 26 or antisense nucleic acids complementary to a POLYX nucleic acid sequence of SEQ ID NO: 1, 3, 5, 7, 9, 11, 13, 15, 17, 19, 21, 23, and 25 are additionally provided.

In one embodiment, an antisense nucleic acid molecule is antisense to a “coding region” of the coding strand of a nucleotide sequence encoding POLY. The term “coding region” refers to the region of the nucleotide sequence comprising codons which are translated into amino acid residues (e.g., the protein coding region of a human POLYX that corresponds to any of SEQ ID NO: 2, 4, 6, 8, 10, 12, 14, 16, 18, 20, 22, 24, and 26). In another embodiment, the antisense nucleic acid molecule is antisense to a “non-coding region” of the coding strand of a nucleotide sequence encoding POLY. The term “non-coding region” refers to 5′ and 3′ sequences which flank the coding region that are not translated into amino acids (i.e., also referred to as 5′ and 3′ non-translated regions).

Given the coding strand sequences encoding POLYX disclosed herein (e.g., SEQ ID NO: 1, 3, 5, 7, 9, 11, 13, 15, 17, 19, 21, 23, and 25), antisense nucleic acids of the invention can be designed according to the rules of Watson and Crick or Hoogsteen base-pairing. The antisense nucleic acid molecule can be complementary to the entire coding region of POLYX mRNA, but more preferably is an oligonucleotide that is antisense to only a portion of the coding or non-coding region of POLYX mRNA. For example, the antisense oligonucleotide can be complementary to the region surrounding the translation start site of POLYX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids, e.g., phosphorothioate derivatives and acridine-substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a POLYX protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual α-units, the strands run parallel to each other (Gaultier, et al., 1987. Nucl. Acids Res. 15: 6625-6641). The antisense nucleic acid molecule can also comprise a 2′-o-methylribonucleotide (Inoue, et al., 1987. Nucl. Acids Res. 15: 6131-6148) or a chimeric RNA-DNA analogue (Inoue, et al., 1987. FEBS Lett. 215: 327-330).

Ribozymes and PNA Moieties

Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In still another embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes; described by Haselhoff and Gerlach, 1988. Nature 334: 585-591) can be used to catalytically-cleave POLYX mRNA transcripts to thereby inhibit translation of POLYX mRNA. A ribozyme having specificity for a POLYX-encoding nucleic acid can be designed based upon the nucleotide sequence of a POLYX DNA disclosed herein (i.e., SEQ ID NO: 1, 3, 5, 7, 9, 11, 13, 15, 17, 19, 21, 23, and 25). For example, a derivative of a Tetrahymena L-19IVS RNA can be constructed in which the nucleotide sequence of the active site is complementary to the nucleotide sequence to be cleaved in a POLYX-encoding mRNA. See, e.g., Cech, et al., U.S. Pat. No. 4,987,071; and Cech, et al., U.S. Pat. No. 5,116,742. Alternatively, POLYX mRNA can be used to select a catalytic RNA having a specific ribonuclease activity from a pool of RNA molecules (Bartel, et al., 1993. Science 261: 1411-1418).

Alternatively, POLYX gene expression can be inhibited by targeting nucleotide sequences complementary to the regulatory region of the POLYX (e.g., the POLYX promoter and/or enhancers) to form triple helical structures that prevent transcription of the POLYX gene in target cells. See, e.g., Helene, 1991. Anticancer Drug Des. 6: 569-84; Helene, et al., 1992. Ann. N.Y. Acad. Sci. 660: 27-36; and Maher, 1992. Bioassays 14: 807-15.

In various embodiments, the nucleic acids of POLYX can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (Hyrup, et al., 1996. Bioorg. Med. Chem. 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of POLYX can be used in therapeutic and diagnostic applications. For example, PNAs can be used as antisense or antigene agents for sequence-specific modulation of gene expression by, e.g., inducing transcription or translation arrest or inhibiting replication. PNAs of POLYX can also be used, e.g., in the analysis of single base pair mutations in a gene by, e.g., PNA directed PCR clamping; as artificial restriction enzymes when used in combination with other enzymes, e.g., S1 nucleases (see, Hyrup, 1996., supra); or as probes or primers for DNA sequence and hybridization (see, Hyrup, et al., 1996.; Perry-O'Keefe, 1996., supra).

In another embodiment, PNAs of POLYX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of POLYX can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, 1996., supra). The synthesis of PNA-DNA chimeras can be performed as described in Finn, et al., (1996. Nucl. Acids Res. 24: 3357-3363). For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag, et al., 1989. Nucl. Acid Res. 17: 5973-5988). PNA monomers are then coupled in a stepwise manner to produce a chimeric molecule with a 5′ PNA segment and a 3′ DNA segment (see, Finn, et al., 1996., supra). Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

Characterization of POLYX Polypeptides

A polypeptide according to the invention includes a polypeptide including the amino acid sequence of POLYX polypeptides whose sequences are provided in any of SEQ ID NO:2, 4, 6, 8, 10, 12, 14, 16, 18, 20, 22, 24, and 26. The invention also includes a mutant or variant protein any of whose residues may be changed from the corresponding residues shown in SEQ ID NOS:2, 4, 6, 8, 10, 12, 14, 16, 18, 20, 22, 24, and 26 while still encoding a protein that maintains its POLYX activities and physiological functions, or a functional fragment thereof.

In general, a POLYX variant that preserves POLYX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated POLYX proteins, and biologically-active portions thereof, or derivatives, fragments, analogs or homologs thereof. Also provided are polypeptide fragments suitable for use as immunogens to raise anti-POLYX antibodies. In one embodiment, native POLYX proteins can be isolated from cells or tissue sources by an appropriate purification scheme using standard protein purification techniques. In another embodiment, POLYX proteins are produced by recombinant DNA techniques. Alternative to recombinant expression, a POLYX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the POLYX protein is derived, or substantially free from chemical precursors or other chemicals when chemically synthesized. The language “substantially free of cellular material” includes preparations of POLYX proteins in which the protein is separated from cellular components of the cells from which it is isolated or recombinantly-produced. In one embodiment, the language “substantially free of cellular material” includes preparations of POLYX proteins having less than about 30% (by dry weight) of non-POLYX proteins (also referred to herein as a “contaminating protein”), more preferably less than about 20% of non-POLYX proteins, still more preferably less than about 10% of non-POLYX proteins, and most preferably less than about 5% of non-POLYX proteins. When the POLYX protein or biologically-active portion thereof is recombinantly-produced, it is also preferably substantially free of culture medium, i.e., culture medium represents less than about 20%, more preferably less than about 10%, and most preferably less than about 5% of the volume of the POLYX protein preparation.

As utilized herein, the phrase “substantially free of chemical precursors or other chemicals” includes preparations of POLYX protein in which the protein is separated from chemical precursors or other chemicals that are involved in the synthesis of the protein. In one embodiment, the language “substantially free of chemical precursors or other chemicals” includes preparations of POLYX protein having less than about 30% (by dry weight) of chemical precursors or non-POLYX chemicals, more preferably less than about 20% chemical precursors or non-POLYX chemicals, still more preferably less than about 10% chemical precursors or non-POLYX chemicals, and most preferably less than about 5% chemical precursors or non-POLYX chemicals.

Biologically-active portions of a POLYX protein include peptides comprising amino acid sequences sufficiently homologous to or derived from the amino acid sequence of the POLYX protein which include fewer amino acids than the full-length POLYX proteins, and exhibit at least one activity of a POLYX protein. Typically, biologically-active portions comprise a domain or motif with at least one activity of the POLYX protein. A biologically-active portion of a POLYX protein can be a polypeptide which is, for example, 10, 25, 50, 100 or more amino acids in length.

A biologically-active portion of a POLYX protein of the invention may contain at least one of the above-identified conserved domains. Moreover, other biologically active portions, in which other regions of the protein are deleted, can be prepared by recombinant techniques and evaluated for one or more of the functional activities of a native POLYX protein.

In an embodiment, the POLYX protein has an amino acid sequence shown in any of SEQ ID NO: 2, 4, 6, 8, 10, 12, 14, 16, 18, 20, 22, 24, and 26. In other embodiments, the POLYX protein is substantially homologous to any of SEQ ID NO: 2, 4, 6, 8, 10, 12, 14, 16, 18, 20, 22, 24, and 26 and retains the functional activity of the protein of any of SEQ ID NO: 2, 4, 6, 8, 10, 12, 14, 16, 18, 20, 22, 24, and 26, yet differs in amino acid sequence due to natural allelic variation or mutagenesis, as described in detail below. Accordingly, in another embodiment, the POLYX protein is a protein that comprises an amino acid sequence at least about 45% homologous, and more preferably about 55, 65, 70, 75, 80, 85, 90, 95, 98 or even 99% homologous to the amino acid sequence of any of SEQ ID NO: 2, 4, 6, 8, 10, 12, 14, 16, 18, 20, 22, 24, and 26 and retains the functional activity of the POLYX proteins of the corresponding polypeptide having the sequence of SEQ ID NO: 2, 4, 6, 8, 10, 12, 14, 16, 18, 20, 22, 24, and 26.

Determining Homology between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J. Mol. Biol. 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NO:1, 3, 5, 7, 9, 11, 13, 15, 17, 19, 21, 23, and 25.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides POLYX chimeric or fusion proteins. As used herein, a POLYX “chimeric protein” or “fusion protein” comprises a POLYX polypeptide operatively-linked to a non-POLYX polypeptide. An “POLYX polypeptide” refers to a polypeptide having an amino acid sequence corresponding to a POLYX protein shown in SEQ ID NO:2, 4, 6, 8, 10, 12, 14, 16, 18, 20, 22, 24, 26 and 62, whereas a “non-POLYX polypeptide” refers to a polypeptide having an amino acid sequence corresponding to a protein that is not substantially homologous to the POLYX protein (e.g., a protein that is different from the POLYX protein and that is derived from the same or a different organism). Within a POLYX fusion protein the POLYX polypeptide can correspond to all or a portion of a POLYX protein. In one embodiment, a POLYX fusion protein comprises at least one biologically-active portion of a POLYX protein. In another embodiment, a POLYX fusion protein comprises at least two biologically-active portions of a POLYX protein. In yet another embodiment, a POLYX fusion protein comprises at least three biologically-active portions of a POLYX protein. Within the fusion protein, the term “operatively-linked” is intended to indicate that the POLYX polypeptide and the non-POLYX polypeptide are fused in-frame with one another. The non-POLYX polypeptide can be fused to the amino-terminus or carboxyl-terminus of the POLYX polypeptide.

In one embodiment, the fusion protein is a GST-POLYX fusion protein in which the POLYX sequences are fused to the carboxyl-terminus of the GST (glutathione S-transferase) sequences. Such fusion proteins can facilitate the purification of recombinant POLYX polypeptides.

In another embodiment, the fusion protein is a POLYX protein containing a heterologous signal sequence at its amino-terminus. In certain host cells (e.g., mammalian host cells), expression and/or secretion of POLYX can be increased through use of a heterologous signal sequence.

In yet another embodiment, the fusion protein is a POLYX-immunoglobulin fusion protein in which the POLYX sequences are fused to sequences derived from a member of the immunoglobulin protein family. The POLYX-immunoglobulin fusion proteins of the invention can be incorporated into pharmaceutical compositions and administered to a subject to inhibit an interaction between a POLYX ligand and a POLYX protein on the surface of a cell, to thereby suppress POLYX-mediated signal transduction in vivo. The POLYX-immunoglobulin fusion proteins can be used to affect the bioavailability of a POLYX cognate ligand. Inhibition of the POLYX ligand/POLYX interaction may be useful therapeutically for both the treatment of proliferative and differentiative disorders, as well as modulating (e.g., promoting or inhibiting) cell survival. Moreover, the POLYX-immunoglobulin fusion proteins of the invention can be used as immunogens to produce anti-POLYX antibodies in a subject, to purify POLYX ligands, and in screening assays to identify molecules that inhibit the interaction of POLYX with a POLYX ligand.

A POLYX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) Current Protocols in Molecular Biology, John Wiley & Sons, 1992). Moreover, many expression vectors are commercially available that already encode a fusion moiety (e.g., a GST polypeptide). A POLYX-encoding nucleic acid can be cloned into such an expression vector such that the fusion moiety is linked in-frame to the POLYX protein.

POLYX Agonists and Antagonists

The invention also pertains to variants of the POLYX proteins that function as either POLYX agonists (i.e., mimetics) or as POLYX antagonists. Variants of the POLYX protein can be generated by mutagenesis (e.g., discrete point mutation or truncation of the POLYX protein). An agonist of a POLYX protein can retain substantially the same, or a subset of, the biological activities of the naturally-occurring form of a POLYX protein. An antagonist of a POLYX protein can inhibit one or more of the activities of the naturally occurring form of a POLYX protein by, for example, competitively binding to a downstream or upstream member of a cellular signaling cascade which includes the POLYX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the POLYX proteins.

Variants of the POLYX proteins that function as either POLYX agonists (i.e., mimetics) or as POLYX antagonists can be identified by screening combinatorial libraries of mutants (e.g., truncation mutants) of the POLYX proteins for POLYX protein agonist or antagonist activity. In one embodiment, a variegated library of POLYX variants is generated by combinatorial mutagenesis at the nucleic acid level and is encoded by a variegated gene library. A variegated library of POLYX variants can be produced by, for example, enzymatically-ligating a mixture of synthetic oligonucleotides into gene sequences such that a degenerate set of potential POLYX sequences is expressible as individual polypeptides, or alternatively, as a set of larger fusion proteins (e.g., for phage display) containing the set of POLYX sequences therein. There are a variety of methods which can be used to produce libraries of potential POLYX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential POLYX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the POLYX protein coding sequences can be used to generate a variegated population of POLYX fragments for screening and subsequent selection of variants of a POLYX protein. In one embodiment, a library of coding sequence fragments can be generated by treating a double-stranded PCR fragment of a POLYX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes amino-terminal and internal fragments of various sizes of the POLYX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of POLYX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify POLYX variants. See, e.g., Arkin and Yourvan, 1992. Proc. Natl. Acad. Sci. USA 89: 7811-7815; Delgrave, et al., 1993. Protein Engineering 6:327-331.

Anti-POLYX Antibodies

The invention encompasses antibodies and antibody fragments, such as F_{ab} or (F_{ab})₂, that bind immunospecifically to any of the POLYX polypeptides of said invention.

An isolated POLYX protein, or a portion or fragment thereof, can be used as an immunogen to generate antibodies that bind to POLYX polypeptides using standard techniques for polyclonal and monoclonal antibody preparation. The full-length POLYX proteins can be used or, alternatively, the invention provides antigenic peptide fragments of POLYX proteins for use as immunogens. The antigenic POLYX peptides comprises at least 4 amino acid residues of the amino acid sequence shown in SEQ ID NO: 2, 4, 6, 8, 10, 12, 14, 16, 18, 20, 22, 24, and 26 and encompasses an epitope of POLYX such that an antibody raised against the peptide forms a specific immune complex with POLY. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of POLYX that is located on the surface of the protein (e.g., a hydrophilic region). As a means for targeting antibody production, hydropathy plots showing regions of hydrophilicity and hydrophobicity may be generated by any method well known in the art, including, for example, the Kyte-Doolittle or the Hopp-Woods methods, either with or without Fourier transformation (see, e.g., Hopp and Woods, 1981. Proc. Nat. Acad. Sci. USA 78: 3824-3828; Kyte and Doolittle, 1982. J. Mol. Biol. 157: 105-142, each incorporated herein by reference in their entirety).

As disclosed herein, POLYX protein sequences of SEQ ID NO: 2, 4, 6, 8, 10, 12, 14, 16, 18, 20, 22, 24, and 26, or derivatives, fragments, analogs, or homologs thereof, may be utilized as immunogens in the generation of antibodies that immunospecifically-bind these protein components. The term “antibody” as used herein refers to immunoglobulin molecules and immunologically-active portions of immunoglobulin molecules, i.e., molecules that contain an antigen binding site that specifically-binds (immunoreacts with) an antigen, such as POLY. Such antibodies include, but are not limited to, polyclonal, monoclonal, chimeric, single chain, F_{ab} and F_{(ab′)2} fragments, and an F_{ab} expression library. In a specific embodiment, antibodies to human POLYX proteins are disclosed. Various procedures known within the art may be used for the production of polyclonal or monoclonal antibodies to a POLYX protein sequence of SEQ ID NO: 2, 4, 6, 8, 10, 12, 14, 16, 18, 20, 22, 24, and 26, or a derivative, fragment, analog, or homolog thereof. Some of these proteins are discussed, infra.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly-expressed POLYX protein or a chemically-synthesized POLYX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against POLYX can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of POLY. A monoclonal antibody composition thus typically displays a single binding affinity for a particular POLYX protein with which it immunoreacts. For preparation of monoclonal antibodies directed towards a particular POLYX protein, or derivatives, fragments, analogs or homologs thereof, any technique that provides for the production of antibody molecules by continuous cell line culture may be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al., 1983. Immunol. Today 4: 72) and the EBV hybridoma technique to produce human monoclonal antibodies (see, e.g., Cole, et al., 1985. In: Molecular Antibodies and Cancer Therapy, Alan R. Liss, Inc., pp. 77-96). Human monoclonal antibodies may be utilized in the practice of the invention and may be produced by using human hybridomas (see, e.g., Cote, et al., 1983. Proc. Natl. Acad. Sci. USA 80: 2026-2030) or by transforming human B-cells with Epstein Barr Virus in vitro (see, e.g., Cole, et al., 1985. In: Monoclonal Antibodies and Cancer Therapy, Alan R. Liss, Inc., pp. 77-96). Each of the above citations is incorporated herein by reference in their entirety.

According to the invention, techniques can be adapted for the production of single-chain antibodies specific to a POLYX protein (see, e.g., U.S. Pat. No. 4,946,778). In addition, methods can be adapted for the construction of F_{ab} expression libraries (see, e.g., Huse, et al., 1989. Science 246: 1275-1281) to allow rapid and effective identification of monoclonal F_{ab} fragments with the desired specificity for a POLYX protein or derivatives, fragments, analogs or homologs thereof. Non-human antibodies can be “humanized” by techniques well known in the art. See, e.g., U.S. Pat. No. 5,225,539. Antibody fragments that contain the idiotypes to a POLYX protein may be produced by techniques known in the art including, but not limited to: (i) an F_{(ab′)2} fragment produced by pepsin digestion of an antibody molecule; (ii) an Fab fragment generated by reducing the disulfide bridges of an F_{(ab′)2} fragment; (iii) an F_{ab} fragment generated by the treatment of the antibody molecule with papain and a reducing agent and (iv) Fᵥ fragments.

Additionally, recombinant anti-POLYX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. No. 4,816,567; U.S. Pat. No. 5,225,539; European Patent Application No. 125,023; Better, et al., 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314:446-449; Shaw, et al., 1988. J. Natl. Cancer Inst. 80: 1553-1559); Morrison(1985) Science 229:1202-1207; Oi, et al. (1986) BioTechniques 4:214; Jones, et al., 1986. Nature 321: 552-525; Verhoeyan, et al., 1988. Science 239: 1534; and Beidler, et al., 1988. J. Immunol. 141: 4053-4060. Each of the above citations are incorporated herein by reference in their entirety.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of a POLYX protein is facilitated by generation of hybridomas that bind to the fragment of a POLYX protein possessing such a domain. Thus, antibodies that are specific for a desired domain within a POLYX protein, or derivatives, fragments, analogs or homologs thereof, are also provided herein.

Anti-POLYX antibodies may be used in methods known within the art relating to the localization and/or quantitation of a POLYX protein (e.g., for use in measuring levels of the POLYX protein within appropriate physiological samples, for use in diagnostic methods, for use in imaging the protein, and the like). In a given embodiment, antibodies for POLYX proteins, or derivatives, fragments, analogs or homologs thereof, that contain the antibody derived binding domain, are utilized as pharmacologically-active compounds (hereinafter “Therapeutics”).

An anti-POLYX antibody (e.g., monoclonal antibody) can be used to isolate a POLYX polypeptide by standard techniques, such as affinity chromatography or immunoprecipitation. An anti-POLYX antibody can facilitate the purification of natural POLYX polypeptide from cells and of recombinantly-produced POLYX polypeptide expressed in host cells. Moreover, an anti-POLYX antibody can be used to detect POLYX protein (e.g., in a cellular lysate or cell supernatant) in order to evaluate the abundance and pattern of expression of the POLYX protein. Anti-POLYX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

POLYX Recombinant Expression Vectors and Host Cells

Another aspect of the invention pertains to vectors, preferably expression vectors, containing a nucleic acid encoding a POLYX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present Specification, “plasmid” and “vector” can be used interchangeably, as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

As utilized herein, the phrase “regulatory sequence” is intended to includes promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., POLYX proteins, mutant forms of POLYX proteins, fusion proteins, etc.).

The recombinant expression vectors of the invention can be designed for expression of POLYX proteins in prokaryotic or eukaryotic cells. For example, POLYX proteins can be expressed in bacterial cells such as Escherichia coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T₇ promoter regulatory sequences and T₇ polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xₐ, thrombin, and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion Escherichia coli expression vectors include pTrc (Amrann et al., (1988) Gene 69:301-315) and pET 11d (Studier, et al., Gene Expression Technology: Methods in Enzymology 185, Academic Press, San Diego, Calif. (1990) 60-89).

One strategy to maximize recombinant protein expression in Escherichia coli is to express the protein in a host bacteria with an impaired capacity to proteolytically-cleave the recombinant protein. See, e.g., Gottesman, Gene Expression Technology: Methods in Enzymology 185, Academic Press, San Diego, Calif. (1990) 119-128. Another strategy is to alter the nucleic acid sequence of the nucleic acid to be inserted into an expression vector so that the individual codons for each amino acid are those preferentially utilized in Escherichia coli (see, e.g., Wada, et al., 1992. Nucl. Acids Res. 20: 2111-2118). Such alteration of nucleic acid sequences of the invention can be carried out by standard DNA synthesis techniques.

In another embodiment, the POLYX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, POLYX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; see, Pinkert, et al., 1987. Genes Dev. 1: 268-277), lymphoid-specific promoters (see, Calame and Eaton, 1988. Adv. Immunol. 43: 235-275), in particular promoters of T cell receptors (see, Winoto and Baltimore, 1989. EMBO J. 8: 729-733) and immunoglobulins (see, Banerji, et al., 1983. Cell 33: 729-740; Queen and Baltimore, 1983. Cell 33: 741-748), neuron-specific promoters (e.g., the neurofilament promoter; see, Byrne and Ruddle, 1989. Proc. Natl. Acad. Sci. USA 86: 5473-5477), pancreas-specific promoters (see, Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (see, Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to POLYX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, POLYX protein can be expressed in bacterial cells such as Escherichia coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin, and methotrexate. Nucleic acid encoding a selectable marker can be introduced into a host cell on the same vector as that encoding POLYX or can be introduced on a separate vector. Cells stably-transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) POLYX protein. Accordingly, the invention further provides methods for producing POLYX protein using the host cells of the invention. In one embodiment, the method comprises culturing the host cell of invention (i.e., into which a recombinant expression vector encoding POLYX protein has been introduced) in a suitable medium such that POLYX protein is produced. In another embodiment, the method further comprises isolating POLYX protein from the medium or the host cell.

Transgenic Animals

The host cells of the invention can also be used to produce non-human transgenic animals. For example, in one embodiment, a host cell of the invention is a fertilized oocyte or an embryonic stem cell into which POLYX protein-coding sequences have been introduced. These host cells can then be used to create non-human transgenic animals in which exogenous POLYX sequences have been introduced into their genome or homologous recombinant animals in which endogenous POLYX sequences have been altered. Such animals are useful for studying the function and/or activity of POLYX protein and for identifying and/or evaluating modulators of POLYX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc.

A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous POLYX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing POLYX-encoding nucleic acid into the male pronuclei of a fertilized oocyte (e.g., by micro-injection, retroviral infection) and allowing the oocyte to develop in a pseudopregnant female foster animal. The human POLYX cDNA sequences of SEQ ID NO: 1, 3, 5, 7, 9, 11, 13, 15, 17, 19, 21, 23, and 25, can be introduced as a transgene into the genome of a non-human animal. Alternatively, a non-human homologue of the human POLYX gene, such as a mouse POLYX gene, can be isolated based on hybridization to the human POLYX cDNA (described further supra) and used as a transgene. Intronic sequences and polyadenylation signals can also be included in the transgene to increase the efficiency of expression of the transgene. A tissue-specific regulatory sequence(s) can be operably-linked to the POLYX transgene to direct expression of POLYX protein to particular cells. Methods for generating transgenic animals via embryo manipulation and micro-injection, particularly animals such as mice, have become conventional in the art and are described, for example, in U.S. Pat. Nos. 4,736,866; 4,870,009; and 4,873,191; and Hogan, 1986. In: Manipulating the Mouse Embryo, Cold Spring Harbor Laboratory Press, Cold Spring Harbor, N.Y. Similar methods are used for production of other transgenic animals. A transgenic founder animal can be identified based upon the presence of the POLYX transgene in its genome and/or expression of POLYX mRNA in tissues or cells of the animals. A transgenic founder animal can then be used to breed additional animals carrying the transgene. Moreover, transgenic animals carrying a transgene-encoding POLYX protein can further be bred to other transgenic animals carrying other transgenes.

To create a homologous recombinant animal, a vector is prepared which contains at least a portion of a POLYX gene into which a deletion, addition or substitution has been introduced to thereby alter, e.g., functionally disrupt, the POLYX gene. The POLYX gene can be a human gene (e.g., the cDNA of SEQ ID NO: 1, 3, 5, 7, 9, 11, 13, 15, 17, 19, 21, 23, and 25), but more preferably, is a non-human homologue of a human POLYX gene. For example, a mouse homologue of human POLYX gene of SEQ ID NO: 1, 3, 5, 7, 9, 11, 13, 15, 17, 19, 21, 23, and 25, can be used to construct a homologous recombination vector suitable for altering an endogenous POLYX gene in the mouse genome. In one embodiment, the vector is designed such that, upon homologous recombination, the endogenous POLYX gene is functionally disrupted (i.e., no longer encodes a functional protein; also referred to as a “knock out” vector).

Alternatively, the vector can be designed such that, upon homologous recombination, the endogenous POLYX gene is mutated or otherwise altered but still encodes functional protein (e.g., the upstream regulatory region can be altered to thereby alter the expression of the endogenous POLYX protein). In the homologous recombination vector, the altered portion of the POLYX gene is flanked at its 5′- and 3′-termini by additional nucleic acid of the POLYX gene to allow for homologous recombination to occur between the exogenous POLYX gene carried by the vector and an endogenous POLYX gene in an embryonic stem cell. The additional flanking POLYX nucleic acid is of sufficient length for successful homologous recombination with the endogenous gene. Typically, several kilobases (Kb) of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced POLYX gene has homologously-recombined with the endogenous POLYX gene are selected. See, e.g., Li, et al., 1992. Cell 69: 915.

The selected cells are then micro-injected into a blastocyst of an animal (e.g., a mouse) to form aggregation chimeras. See, e.g., Bradley, 1987. In: Teratocarcinomas and Embryonic Stem Cells: A Practical Approach, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-human animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte, and then transferred to pseudopregnant female foster animal. The offspring borne of this female foster animal will be a clone of the animal from which the cell (e.g., the somatic cell) is isolated.

Pharmaceutical Compositions

The POLYX nucleic acid molecules, POLYX proteins, and anti-POLYX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically-acceptable carrier. As used herein, “pharmaceutically-acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and other non-aqueous (i.e., lipophilic) vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a POLYX protein or anti-POLYX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The nucleic acid molecules, proteins, protein homologues, and antibodies described herein can be used in one or more of the following methods: (i) screening assays; (ii) detection assays (e.g., chromosomal mapping, cell and tissue typing, forensic biology), (iii) predictive medicine (e.g., diagnostic assays, prognostic assays, monitoring clinical trials, and pharmacogenomics); and (iv) methods of treatment (e.g., therapeutic and prophylactic).

The isolated nucleic acid molecules of the present invention can be used to express POLYX protein (e.g., via a recombinant expression vector in a host cell in gene therapy applications), to detect POLYX mRNA (e.g., in a biological sample) or a genetic lesion in an POLYX gene, and to modulate POLYX activity, as described further, infra. In addition, the POLYX proteins can be used to screen drugs or compounds that modulate the POLYX protein activity or expression as well as to treat disorders characterized by insufficient or excessive production of POLYX protein or production of POLYX protein forms that have decreased or aberrant activity compared to POLYX wild-type protein. In addition, the anti-POLYX antibodies of the present invention can be used to detect and isolate POLYX proteins and modulate POLYX activity.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to POLYX proteins or have a stimulatory or inhibitory effect on, e.g., POLYX protein expression or POLYX protein activity. The invention also includes compounds identified in the screening assays described herein.

In one embodiment, the invention provides assays for screening candidate or test compounds which bind to or modulate the activity of the membrane-bound form of a POLYX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222:301-310; Ladner, U.S. Pat. No. 5,233,409.).

In one embodiment, an assay is a cell-based assay in which a cell which expresses a membrane-bound form of POLYX protein, or a biologically-active portion thereof, on the cell surface is contacted with a test compound and the ability of the test compound to bind to a POLYX protein determined. The cell, for example, can of mammalian origin or a yeast cell. Determining the ability of the test compound to bind to the POLYX protein can be accomplished, for example, by coupling the test compound with a radioisotope or enzymatic label such that binding of the test compound to the POLYX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of POLYX protein, or a biologically-active portion thereof, on the cell surface with a known compound which binds POLYX to form an assay mixture, contacting the assay mixture with a test compound, and determining the ability of the test compound to interact with a POLYX protein, wherein determining the ability of the test compound to interact with a POLYX protein comprises determining the ability of the test compound to preferentially bind to POLYX protein or a biologically-active portion thereof as compared to the known compound.

In another embodiment, an assay is a cell-based assay comprising contacting a cell expressing a membrane-bound form of POLYX protein, or a biologically-active portion thereof, on the cell surface with a test compound and determining the ability of the test compound to modulate (e.g., stimulate or inhibit) the activity of the POLYX protein or biologically-active portion thereof. Determining the ability of the test compound to modulate the activity of POLYX or a biologically-active portion thereof can be accomplished, for example, by determining the ability of the POLYX protein to bind to or interact with a POLYX target molecule. As used herein, a “target molecule” is a molecule with which a POLYX protein binds or interacts in nature, for example, a molecule on the surface of a cell which expresses a POLYX interacting protein, a molecule on the surface of a second cell, a molecule in the extracellular milieu, a molecule associated with the internal surface of a cell membrane or a cytoplasmic molecule. An POLYX target molecule can be a non-POLYX molecule or a POLYX protein or polypeptide of the invention. In one embodiment, a POLYX target molecule is a component of a signal transduction pathway that facilitates transduction of an extracellular signal (e.g. a signal generated by binding of a compound to a membrane-bound POLYX molecule) through the cell membrane and into the cell. The target, for example, can be a second intercellular protein that has catalytic activity or a protein that facilitates the association of downstream signaling molecules with POLY.

Determining the ability of the POLYX protein to bind to or interact with a POLYX target molecule can be accomplished by one of the methods described above for determining direct binding. In one embodiment, determining the ability of the POLYX protein to bind to or interact with a POLYX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a POLYX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a POLYX protein or biologically-active portion thereof with a test compound and determining the ability of the test compound to bind to the POLYX protein or biologically-active portion thereof. Binding of the test compound to the POLYX protein can be determined either directly or indirectly as described above. In one such embodiment, the assay comprises contacting the POLYX protein or biologically-active portion thereof with a known compound which binds POLYX to form an assay mixture, contacting the assay mixture with a test compound, and determining the ability of the test compound to interact with a POLYX protein, wherein determining the ability of the test compound to interact with a POLYX protein comprises determining the ability of the test compound to preferentially bind to POLYX or biologically-active portion thereof as compared to the known compound.

In still another embodiment, an assay is a cell-free assay comprising contacting POLYX protein or biologically-active portion thereof with a test compound and determining the ability of the test compound to modulate (e.g. stimulate or inhibit) the activity of the POLYX protein or biologically-active portion thereof. Determining the ability of the test compound to modulate the activity of POLYX can be accomplished, for example, by determining the ability of the POLYX protein to bind to a POLYX target molecule by one of the methods described above for determining direct binding. In an alternative embodiment, determining the ability of the test compound to modulate the activity of POLYX protein can be accomplished by determining the ability of the POLYX protein further modulate a POLYX target molecule. For example, the catalytic/enzymatic activity of the target molecule on an appropriate substrate can be determined as described, supra.

In yet another embodiment, the cell-free assay comprises contacting the POLYX protein or biologically-active portion thereof with a known compound which binds POLYX protein to form an assay mixture, contacting the assay mixture with a test compound, and determining the ability of the test compound to interact with a POLYX protein, wherein determining the ability of the test compound to interact with a POLYX protein comprises determining the ability of the POLYX protein to preferentially bind to or modulate the activity of a POLYX target molecule.

The cell-free assays of the invention are amenable to use of both the soluble form or the membrane-bound form of POLYX protein. In the case of cell-free assays comprising the membrane-bound form of POLYX protein, it may be desirable to utilize a solubilizing agent such that the membrane-bound form of POLYX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either POLYX protein or its target molecule to facilitate separation of complexed from uncomplexed forms of one or both of the proteins, as well as to accommodate automation of the assay. Binding of a test compound to POLYX protein, or interaction of POLYX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-POLYX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or POLYX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of POLYX protein binding or activity determined using standard techniques.

Other techniques for immobilizing proteins on matrices can also be used in the screening assays of the invention. For example, either the POLYX protein or its target molecule can be immobilized utilizing conjugation of biotin and streptavidin. Biotinylated POLYX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with POLYX protein or target molecules, but which do not interfere with binding of the POLYX protein to its target molecule, can be derivatized to the wells of the plate, and unbound target or POLYX protein trapped in the wells by antibody conjugation. Methods for detecting such complexes, in addition to those described above for the GST-immobilized complexes, include immunodetection of complexes using antibodies reactive with the POLYX protein or target molecule, as well as enzyme-linked assays that rely on detecting an enzymatic activity associated with the POLYX protein or target molecule.

In another embodiment, modulators of POLYX protein expression are identified in a method wherein a cell is contacted with a candidate compound and the expression of POLYX mRNA or protein in the cell is determined. The level of expression of POLYX mRNA or protein in the presence of the candidate compound is compared to the level of expression of POLYX mRNA or protein in the absence of the candidate compound. The candidate compound can then be identified as a modulator of POLYX mRNA or protein expression based upon this comparison. For example, when expression of POLYX mRNA or protein is greater (i.e., statistically significantly greater) in the presence of the candidate compound than in its absence, the candidate compound is identified as a stimulator of POLYX mRNA or protein expression. Alternatively, when expression of POLYX mRNA or protein is less (statistically significantly less) in the presence of the candidate compound than in its absence, the candidate compound is identified as an inhibitor of POLYX mRNA or protein expression. The level of POLYX mRNA or protein expression in the cells can be determined by methods described herein for detecting POLYX mRNA or protein.

In yet another aspect of the invention, the POLYX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with POLYX (“POLYX-binding proteins” or “POLYX-bp”) and modulate POLYX activity. Such POLYX-binding proteins are also likely to be involved in the propagation of signals by the POLYX proteins as, for example, upstream or downstream elements of the POLYX pathway.

The two-hybrid system is based on the modular nature of most transcription factors, which consist of separable DNA-binding and activation domains. Briefly, the assay utilizes two different DNA constructs. In one construct, the gene that codes for POLYX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a POLYX-dependent complex, the DNA-binding and activation domains of the transcription factor are brought into close POLYX imity. This POLYX imity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with POLY.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, infra.

Chromosome Mapping

Once the sequence (or a portion of the sequence) of a gene has been isolated, this sequence can be used to map the location of the gene on a chromosome. This process is called chromosome mapping. Accordingly, portions or fragments of the POLYX sequences shown in SEQ ID NO: 1, 3, 5, 7, 9, 11, 13, 15, 17, 19, 21, 23, and 25, or fragments or derivatives thereof, can be used to map the location of the POLYX genes, respectively, on a chromosome. The mapping of the POLYX sequences to chromosomes is an important first step in correlating these sequences with genes associated with disease.

Briefly, POLYX genes can be mapped to chromosomes by preparing PCR primers (preferably 15-25 bp in length) from the POLYX sequences. Computer analysis of the POLY, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the POLYX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the POLYX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., Human Chromosomes: A Manual of Basic techniques (Pergamon Press, NY 1988).

Reagents for chromosome mapping can be used individually to mark a single chromosome or a single site on that chromosome, or panels of reagents can be used for marking multiple sites and/or multiple chromosomes. Reagents corresponding to non-coding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Additionally, differences in the DNA sequences between individuals affected and unaffected with a disease associated with the POLYX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The POLYX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” as described in U.S. Pat. No. 5,272,057).

Furthermore, the sequences of the invention can be used to provide an alternative technique that determines the actual base-by-base DNA sequence of selected portions of an individual's genome. Thus, the POLYX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The POLYX sequences of the invention uniquely represent portions of the human genome. Allelic variation occurs to some degree in the coding regions of these sequences, and to a greater degree in the non-coding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the non-coding regions, fewer sequences are necessary to differentiate individuals. The non-coding sequences can comfortably provide positive individual identification with a panel of perhaps 10 to 1,000 primers that each yield a non-coding amplified sequence of 100 bases. If predicted coding sequences, such as those in SEQ ID NO: 1, 3, 5, 7, 9, 11, 13, 15, 17, 19, 21, 23, and 25 are used, a more appropriate number of primers for positive individual identification would be 500-2,000.

Use of Partial POLYX Sequences in Forensic Biology

DNA-based identification techniques can also be used in forensic biology. Forensic biology is a scientific field employing genetic typing of biological evidence found at a crime scene as a means for positively identifying, e.g., a perpetrator of a crime. To make such an identification, PCR technology can be used to amplify DNA sequences taken from very small biological samples such as tissues (e.g., hair or skin, or body fluids, e.g., blood, saliva, or semen found at a crime scene). The amplified sequence can then be compared to a standard, thereby allowing identification of the origin of the biological sample.

The sequences of the invention can be used to provide polynucleotide reagents, e.g., PCR primers, targeted to specific loci in the human genome, that can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to non-coding regions of SEQ ID NO: 1, 3, 5, 7, 9, 11, 13, 15, 17, 19, 21, 23, and 25 are particularly appropriate for this use as greater numbers of polymorphisms occur in the non-coding regions, making it easier to differentiate individuals using this technique. Examples of polynucleotide reagents include the POLYX sequences or portions thereof, e.g., fragments derived from the non-coding regions of one or more of SEQ ID NO: 1, 3, 5, 7, 9, 11, 13, 15, 17, 19, 21, 23, and 25, having a length of at least 20 bases, preferably at least 30 bases.

The POLYX sequences described herein can further be used to provide polynucleotide reagents, e.g., labeled or label-able probes that can be used, for example, in an in situ hybridization technique, to identify a specific tissue (e.g., brain tissue, etc). This can be very useful in cases where a forensic pathologist is presented with a tissue of unknown origin. Panels of such POLYX probes can be used to identify tissue by species and/or by organ type.

In a similar fashion, these reagents, e.g., POLYX primers or probes can be used to screen tissue culture for contamination (i.e., screen for the presence of a mixture of different types of cells in a culture).

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining POLYX protein and/or nucleic acid expression as well as POLYX activity, in the context of a biological sample (e.g., blood, serum, cells, tissue) to thereby determine whether an individual is afflicted with a disease or disorder, or is at risk of developing a disorder, associated with aberrant POLYX expression or activity. The invention also provides for prognostic (or predictive) assays for determining whether an individual is at risk of developing a disorder associated with POLYX protein, nucleic acid expression or activity. For example, mutations in a POLYX gene can be assayed in a biological sample. Such assays can be used for prognostic or predictive purpose to thereby prophylactically treat an individual prior to the onset of a disorder characterized by or associated with POLYX protein, nucleic acid expression, or biological activity.

Another aspect of the invention provides methods for determining POLYX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of POLYX in clinical trials. These and other agents are described in further detail in the following sections.

Diagnostic Assays

An exemplary method for detecting the presence or absence of POLYX in a biological sample involves obtaining a biological sample from a test subject and contacting the biological sample with a compound or an agent capable of detecting POLYX protein or nucleic acid (e.g., mRNA, genomic DNA) that encodes POLYX protein such that the presence of POLYX is detected in the biological sample. An agent for detecting POLYX mRNA or genomic DNA is a labeled nucleic acid probe capable of hybridizing to POLYX mRNA or genomic DNA. The nucleic acid probe can be, for example, a full-length POLYX nucleic acid, such as the nucleic acid of SEQ ID NO: 1, 3, 5, 7, 9, 11, 13, 15, 17, 19, 21, 23, and 25, or a portion thereof, such as an oligonucleotide of at least 15, 30, 50, 100, 250 or 500 nucleotides in length and sufficient to specifically hybridize under stringent conditions to POLYX mRNA or genomic DNA. Other suitable probes for use in the diagnostic assays of the invention are described herein.

An agent for detecting POLYX protein is an antibody capable of binding to POLYX protein, preferably an antibody with a detectable label. Antibodies can be polyclonal, or more preferably, monoclonal. An intact antibody, or a fragment thereof (e.g., F_{ab} or F_{(ab)2}) can be used. As utilized herein, the term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. As utilized herein, the term “biological sample” is intended to include tissues, cells and biological fluids isolated from a subject, as well as tissues, cells and fluids present within a subject. That is, the detection method of the invention can be used to detect POLYX mRNA, protein, or genomic DNA in a biological sample in vitro as well as in vivo. For example, in vitro techniques for detection of POLYX mRNA include Northern hybridizations and in situ hybridizations. In vitro techniques for detection of POLYX protein include enzyme linked immunosorbent assays (ELISAs), Western blots, immunoprecipitations, and immunofluorescence. In vitro techniques for detection of POLYX genomic DNA include Southern hybridizations. Furthermore, in vivo techniques for detection of POLYX protein include introducing into a subject a labeled anti-POLYX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting POLYX protein, mRNA, or genomic DNA, such that the presence of POLYX protein, mRNA or genomic DNA is detected in the biological sample, and comparing the presence of POLYX protein, mRNA or genomic DNA in the control sample with the presence of POLYX protein, mRNA or genomic DNA in the test sample.

The invention also encompasses kits for detecting the presence of POLYX in a biological sample. For example, the kit can comprise: a labeled compound or agent capable of detecting POLYX protein or mRNA in a biological sample; means for determining the amount of POLYX in the sample; and means for comparing the amount of POLYX in the sample with a standard. The compound or agent can be packaged in a suitable container. The kit can further comprise instructions for using the kit to detect POLYX protein or nucleic acid.

Prognostic Assays

The diagnostic methods described herein can furthermore be utilized to identify subjects having or at risk of developing a disease or disorder associated with aberrant POLYX expression or activity. For example, the assays described herein, such as the preceding diagnostic assays or the following assays, can be utilized to identify a subject having or at risk of developing a disorder associated with POLYX protein, nucleic acid expression or activity. Alternatively, the prognostic assays can be utilized to identify a subject having or at risk for developing a disease or disorder. Thus, the invention provides a method for identifying a disease or disorder associated with aberrant POLYX expression or activity in which a test sample is obtained from a subject and POLYX protein or nucleic acid (e.g., mRNA, genomic DNA) is detected, wherein the presence of POLYX protein or nucleic acid is diagnostic for a subject having or at risk of developing a disease or disorder associated with aberrant POLYX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant POLYX expression or activity. For example, such methods can be used to determine whether a subject can be effectively treated with an agent for a disorder. Thus, the invention provides methods for determining whether a subject can be effectively treated with an agent for a disorder associated with aberrant POLYX expression or activity in which a test sample is obtained and POLYX protein or nucleic acid is detected (e.g., wherein the presence of POLYX protein or nucleic acid is diagnostic for a subject that can be administered the agent to treat a disorder associated with aberrant POLYX expression or activity).

The methods of the invention can also be used to detect genetic lesions in a POLYX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a POLYX-protein, or the mis-expression of the POLYX gene. For example, such genetic lesions can be detected by ascertaining the existence of at least one of: (i) a deletion of one or more nucleotides from a POLYX gene; (ii) an addition of one or more nucleotides to a POLYX gene; (iii) a substitution of one or more nucleotides of a POLYX gene, (iv) a chromosomal rearrangement of a POLYX gene; (v) an alteration in the level of a messenger RNA transcript of a POLYX gene; (vi) aberrant modification of a POLYX gene, such as of the methylation pattern of the genomic DNA; (vii) the presence of a non-wild-type splicing pattern of a messenger RNA transcript of a POLYX gene; (viii) a non-wild-type level of a POLYX protein, (ix) allelic loss of a POLYX gene; and (x) inappropriate post-translational modification of a POLYX protein. As described herein, there are a large number of assay techniques known in the art which can be used for detecting lesions in a POLYX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the POLYX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a POLYX gene under conditions such that hybridization and amplification of the POLYX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a POLYX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in POLYX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in POLYX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the POLYX gene and detect mutations by comparing the sequence of the sample POLYX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. BioTechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the POLYX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type POLYX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in POLYX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a POLYX sequence, e.g., a wild-type POLYX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in POLYX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control POLYX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of apPOLYXimately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a POLYX gene.

Furthermore, any cell type or tissue, preferably peripheral blood leukocytes, in which POLYX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on POLYX activity (e.g., POLYX gene expression), as identified by a screening assay described herein can be administered to individuals to treat (prophylactically or therapeutically) disorders (e.g., cancer or immune disorders associated with aberrant POLYX activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of POLYX protein, expression of POLYX nucleic acid, or mutation content of POLYX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol. 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of POLYX protein, expression of POLYX nucleic acid, or mutation content of POLYX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a POLYX modulator, such as a modulator identified by one of the exemplary screening assays described herein.

Monitoring of Effects During Clinical Trials

Monitoring the influence of agents (e.g., drugs, compounds) on the expression or activity of POLYX (e.g., the ability to modulate aberrant cell proliferation and/or differentiation) can be applied not only in basic drug screening, but also in clinical trials. For example, the effectiveness of an agent determined by a screening assay as described herein to increase POLYX gene expression, protein levels, or upregulate POLYX activity, can be monitored in clinical trails of subjects exhibiting decreased POLYX gene expression, protein levels, or downregulated POLYX activity. Alternatively, the effectiveness of an agent determined by a screening assay to decrease POLYX gene expression, protein levels, or downregulate POLYX activity, can be monitored in clinical trails of subjects exhibiting increased POLYX gene expression, protein levels, or upregulated POLYX activity. In such clinical trials, the expression or activity of POLYX and, preferably, other genes that have been implicated in, for example, a cellular proliferation or immune disorder can be used as a “read out” or markers of the immune responsiveness of a particular cell.

By way of example, and not of limitation, genes, including POLY, that are modulated in cells by treatment with an agent (e.g., compound, drug or small molecule) that modulates POLYX activity (e.g., identified in a screening assay as described herein) can be identified. Thus, to study the effect of agents on cellular proliferation disorders, for example, in a clinical trial, cells can be isolated and RNA prepared and analyzed for the levels of expression of POLYX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of POLYX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a POLYX protein, mRNA, or genomic DNA in the pre-administration sample; (iii) obtaining one or more post-administration samples from the subject; (iv) detecting the level of expression or activity of the POLYX protein, mRNA, or genomic DNA in the post-administration samples; (v) comparing the level of expression or activity of the POLYX protein, mRNA, or genomic DNA in the pre-administration sample with the POLYX protein, mRNA, or genomic DNA in the post administration sample or samples; and (vi) altering the administration of the agent to the subject accordingly. For example, increased administration of the agent may be desirable to increase the expression or activity of POLYX to higher levels than detected, i.e., to increase the effectiveness of the agent. Alternatively, decreased administration of the agent may be desirable to decrease expression or activity of POLYX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant POLYX expression or activity. These methods of treatment will be discussed more fully, infra.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endoggenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant POLYX expression or activity, by administering to the subject an agent that modulates POLYX expression or at least one POLYX activity. Subjects at risk for a disease that is caused or contributed to by aberrant POLYX expression or activity can be identified by, for example, any or a combination of diagnostic or prognostic assays as described herein. Administration of a prophylactic agent can occur prior to the manifestation of symptoms characteristic of the POLYX aberrancy, such that a disease or disorder is prevented or, alternatively, delayed in its progression. Depending upon the type of POLYX aberrancy, for example, a POLYX agonist or POLYX antagonist agent can be used for treating the subject. The appropriate agent can be determined based on screening assays described herein.

Therapeutic Methods

Another aspect of the invention pertains to methods of modulating POLYX expression or activity for therapeutic purposes. The modulatory method of the invention involves contacting a cell with an agent that modulates one or more of the activities of POLYX protein activity associated with the cell. An agent that modulates POLYX protein activity can be an agent as described herein, such as a nucleic acid or a protein, a naturally-occurring cognate ligand of a POLYX protein, a peptide, a POLYX peptidomimetic, or other small molecule. In one embodiment, the agent stimulates one or more POLYX protein activity. Examples of such stimulatory agents include active POLYX protein and a nucleic acid molecule encoding POLYX that has been introduced into the cell. In another embodiment, the agent inhibits one or more POLYX protein activity. Examples of such inhibitory agents include antisense POLYX nucleic acid molecules and anti-POLYX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a POLYX protein or nucleic acid molecule. In one embodiment, the method involves administering an agent (e.g., an agent identified by a screening assay described herein), or combination of agents that modulates (e.g., up-regulates or down-regulates) POLYX expression or activity. In another embodiment, the method involves administering a POLYX protein or nucleic acid molecule as therapy to compensate for reduced or aberrant POLYX expression or activity.

Stimulation of POLYX activity is desirable in situations in which POLYX is abnormally downregulated and/or in which increased POLYX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., pre-clampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The POLYX nucleic acids and proteins of the invention may be useful in a variety of potential prophylactic and therapeutic applications. By way of a non-limiting example, a cDNA encoding the POLYX protein of the invention may be useful in gene therapy, and the protein may be useful when administered to a subject in need thereof.

Both the novel nucleic acids encoding the POLYX proteins, and the POLYX proteins of the invention, or fragments thereof, may also be useful in diagnostic applications, wherein the presence or amount of the nucleic acid or the protein are to be assessed. These materials are further useful in the generation of antibodies which immunospecifically-bind to the novel substances of the invention for use in therapeutic or diagnostic methods.

The invention will be further illustrated in the following non-limiting examples.

### EXAMPLE 1

### Radiation Hybrid Mapping Provides the Chromosomal Location of Clones, POLYX

Radiation hybrid mapping using human chromosome markers was carried out for many of the clones described in the present invention. The procedure used to obtain these results is analogous to that described in Steen, R G et al. (A High-Density Integrated Genetic Linkage and Radiation Hybrid Map of the Laboratory Rat, Genome Research 1999 (Published Online on May 21, 1999)Vol. 9, AP1-AP8, 1999). A panel of 93 cell clones containing randomized radiation-induced human chromosomal fragments was screened in 96 well plates using PCR primers designed to identify the sought clones in a unique fashion. Table 15 provides the results obtained for three clones of the present invention.

TABLE 15

| | | | Distance from | Distance from |
|---|---|---|---|---|
| POLYX # | Clone | Chromosome | Marker, cR | Marker, cR |
| 4 | 10129612 | 5 | AFMA109XA5, | WI-6737, 2.4 cR |
| | | | 0.60 cR | |
| 6 | 10354784 | 3 | WI-1780, 0.0 cR | |
| 13 | 20468752-0-18 | 11 | WI-6150, 2.8 cR | WI-5256, 3.8 cR |

### EXAMPLE 2

### Molecular Cloning of 10129612-1, POLY4

The predicted open reading frame codes for a novel 429 amino acid long protein predicted to be a TypeII transmembrane protein. The cDNA coding for the extracellular domain of residues 250-429 was targeted for cloning.

Oligonucleotide primers were designed to PCR amplify a DNA segment coding for the extracellular domain of 10129612-1. The forward primer includes an in frame BamHI restriction site and the reverse primer contains an in frame XhoI restriction site. The sequences of the primers are the following:

| 10129612-1 | | |
|---|---|---|
| Forward: | GGA TCC AAC AGC AGC ATA GAC AGT GGT GAA GCA | (SEQ ID NO:27) |
| 10129612-1 | | |
| Reverse: | CTC GAG CAG AGC AGC TTT ATT AAT GAT TGT CTT GCA GAA | (SEQ ID NO:28) |

PCR reactions were set up using 5 ng cDNA template consisting of equal portions of human testis, fetal brain, mammary, and skeletal muscle derived cDNA samples, 1 microM of each of the 10129612-1 Forward and 10129612-1 Reverse primers, 5 micromoles dNTP (Clontech Laboratories, Palo Alto Calif.) and 1 microliter of 50×Advantage-HF 2 polymerase (Clontech Laboratories, Palo Alto Calif.) in 50 microliter volume. The following reaction conditions were used:

a) 96° C. 3 minutes

b) 96° C. 30 seconds denaturation

c) 70° C. 30 seconds, primer annealing. This temperature was gradually decreased by 1° C./cycle

d) 72° C. 1 minutes extension.

Repeat steps b-d 10 times

e) 96° C. 30 seconds denaturation

f) 60° C. 30 seconds annealing

g) 72° C. 1 minutes extension

Repeat steps e-g 35 times

h) 72° C. 5 minutes final extension

A 530 bp large, amplified product was detected by agarose gel electrophoresis. The product was isolated, and ligated into the pCR2.1 vector (Invitrogen, Carlsbad Calif.). The DNA sequence of the cloned insert was determined as an ORF coding for a 180 amino acid long polypeptide that matches the target DNA sequence of 10129612-1 (SEQ ID NO:8) 100%. The construct is called as pCR2.1-cg10129612-S333-6C.

### EXAMPLE 3

### Preparation of Mammalian Expression Vector pCEP4/Sec

Two oligonucleotide primers were designed to amplify a fragment from the pcDNA3.1-V5His (Invitrogen, Carlsbad, Calif.) expression vector that includes V5 and His6. These primers include:

| | |
|---|---|
| pSec-V5-His Forward: | |
| CTCGTCCTCGAGGGTAAGCCTATCCCTAAC | (SEQ ID NO:29) |
| pSec-V5-His Reverse: | |
| CTCGTCGGGCCCCTGATCAGCGGGTTTAAAC | (SEQ ID NO:30) |

Following PCR amplification, the product was digested with XhoI and ApaI and ligated into the XhoI/ApaI-digested pSecTag2 B vector harboring an i kappa leader sequence (Invitrogen; Carlsbad, Calif.). The correct structure of the resulting vector (designated pSecV5His), including an in-frame i-kappa leader and V5-His6, was verified by DNA sequence analysis. The vector pSecV5His was then digested with PmeI and NheI to provide a fragment retaining the above elements in the correct frame. The PmeI/NheI-digested fragment was ligated into the BamHI/Klenow- and NheI-treated vector pCEP4 (Invitrogen; Carlsbad, Calif.). The resulting vector was designated pCEP4/Sec, and included an in-frame i kappa leader, a site for insertion of a clone of interest, and V5 and His6 sites under control of the PCMV and/or the PT7 promoter. pCEP4/Sec is an expression vector that allows heterologous protein expression and secretion by fusing any protein to the i Kappa chain signal peptide. Detection and purification of the expressed protein was aided by the presence of the V5 epitope tag and 6x His tag at the carboxyl-terminus (Invitrogen; Carlsbad, Calif.).

### EXAMPLE 4

### Expression of POLY4, Clone 10129612, in Human Embryonic Kidney 293 Cells

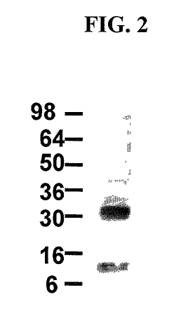

The BamHI-XhoI fragment containing the 10129612 sequence was isolated from pCR2.1-cg10129612-S333-6C (Example AB1) and subcloned into the vector pCEP4/Sec (Example AB2) to generate expression vector pCEP4/Sec-10129612. The pCEP4/Sec-10129612 vector was transfected into 293 cells using the LipofectaminePlus reagent following the manufacturer's instructions (Gibco/BRL). The cell pellet and supernatant were harvested 72 hours after transfection and examined for 10129612 expression by Western blotting (reducing conditions) with an anti-V5 antibody. FIG. 2 shows that 10129612 is expressed as a protein of apparent molecular weight 30 kDa secreted by 293 cells.

### EXAMPLE 5

### Quantitative Analysis of the Tissue Distribution of Expression of the Nucleic Acids of the Invention

The quantitative expression of various clones was assessed in 41 normal and 55 tumor samples (the samples are identified in the Tables that follow) by real time quantitative PCR (TAQMAN®) performed on a Perkin-Elmer Biosystems ABI PRISM® 7700 Sequence Detection System. In these Tables, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

The RNA samples for each cell or tissue were normalized according to RNA input by RNA quantification using Ribogreen (Molecular Probes, Eugene Oreg.; Catalog number R-11491) according to the manufacturer's directions using a standard curve covering 1 ng/ml through 50 ng/ml RNA. RNA quantity input was confirmed by monitoring the expression of human polypeptide chain elongation factor-1 alpha (GenBank Accession Number: E02629) and human ADP-ribosylation factor 1 (ARF1) mRNA (GenBank Accession Number: M36340). RNA (˜50 ng total or ˜1 ng polyA+) was converted to cDNA using the TAQMAN® Reverse Transcription Reagents Kit (PE Biosystems, Foster City, Calif.; cat #N808-0234) and random hexamers according to the manufacturer's protocol. Reactions were performed in 20 μl and incubated for 30 min. at 48° C. cDNA (5 μl) was then transferred to a separate plate for the TAQMAN® reaction using probe and primer sets specific for human polypeptide chain elongation factor-1 alpha and human ADP-ribosylation factor 1 (ARF1) mRNA were designed for each assay according to a proprietary software package. Reactions were carried out using the TAQMAN® universal PCR Master Mix (PE Biosystems; cat #4304447) according to the manufacturer's protocol. Reactions were performed in 25 μl using the following parameters: 2 min. at 50° C.; 10 min. at 95° C.; 15 sec. at 95° C./1 min. at 60° C. (40 cycles). Results were recorded as CT values (cycle at which a given sample crosses a threshold level of fluorescence) using a log scale, with the difference in RNA concentration between a given sample and the sample with the lowest CT value being represented as 2 to the power of delta CT (2^{δCT}). The percent relative expression is then obtained by taking the reciprocal of this RNA difference and multiplying by 100. The average CT values obtained for human polypeptide chain elongation factor-1 alpha and human ADP-ribosylation factor 1 (ARF1) mRNA were used to normalize RNA samples. The RNA sample generating the highest CT value required no further diluting, while all other samples were diluted relative to this sample according to their β-actin/GAPDH average CT values.

Normalized RNA (5 ul) was converted to cDNA and analyzed via TAQMAN® using One Step RT-PCR Master Mix Reagents (PE Biosystems; cat. #4309169) and gene-specific primers according to the manufacturer's instructions. Probes and primers were designed for each assay according to Perkin Elmer Biosystem's Primer Express Software package (version I for Apple Computer's Macintosh Power PC) using the sequence of the clone being analyzed as input. Default settings were used for reaction conditions and the following parameters were set before selecting primers: primer concentration=250 nM, primer melting temperature (Tₘ) range=58°-60° C., primer optimal Tm=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: Normalized RNA from each tissue and each cell line was spotted in each well of a 96 well PCR plate (Perkin Elmer Biosystems). PCR cocktails including two probes (POLYX-specific and another gene-specific probe multiplexed with the POLYX probe) were set up using 1×TaqMan™ PCR Master Mix for the PE Biosystems 7700, with 5 mM MgCl2, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute.

In the following presentation, the Tables provide the sequences used for the primers and the probe, and the relative expression results obtained for the cell cultures employed are shown in various Figures and Tables.

a) Clone 10129612, POLY4

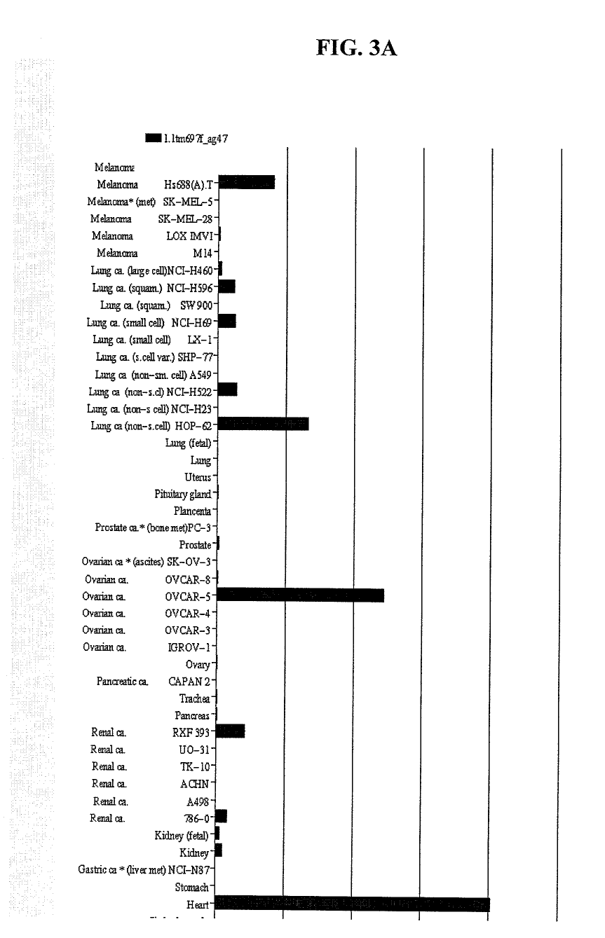

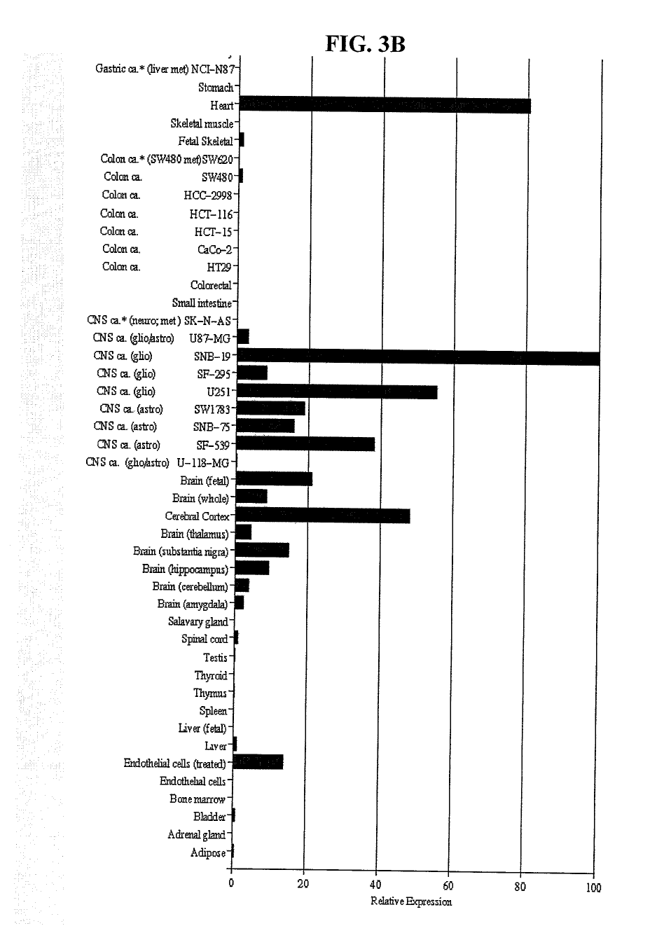

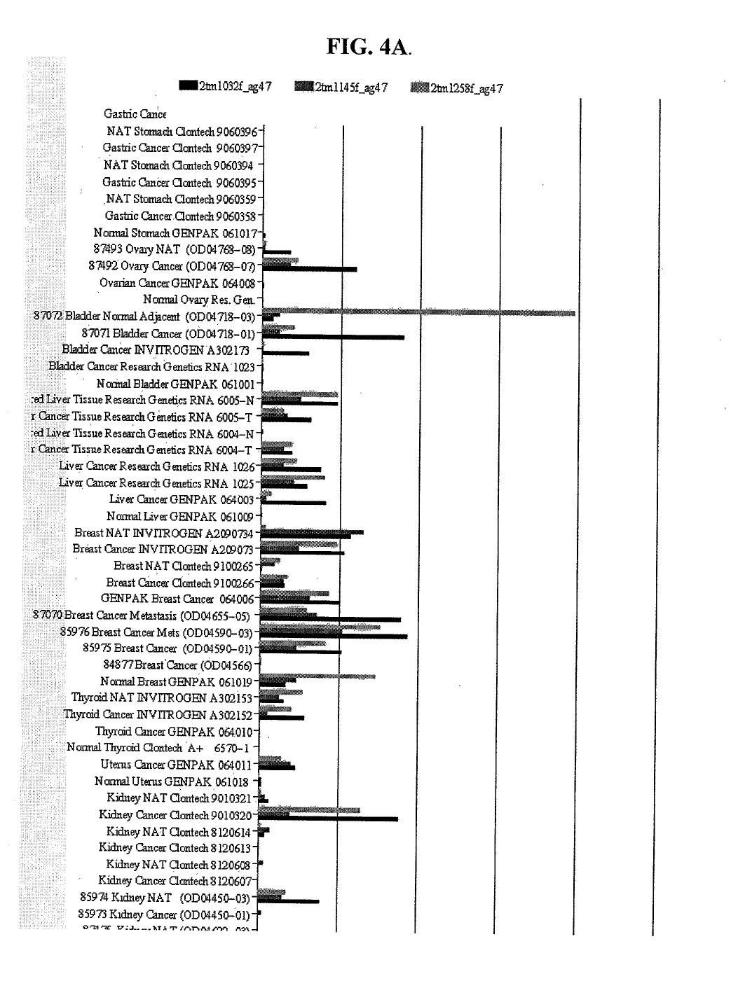

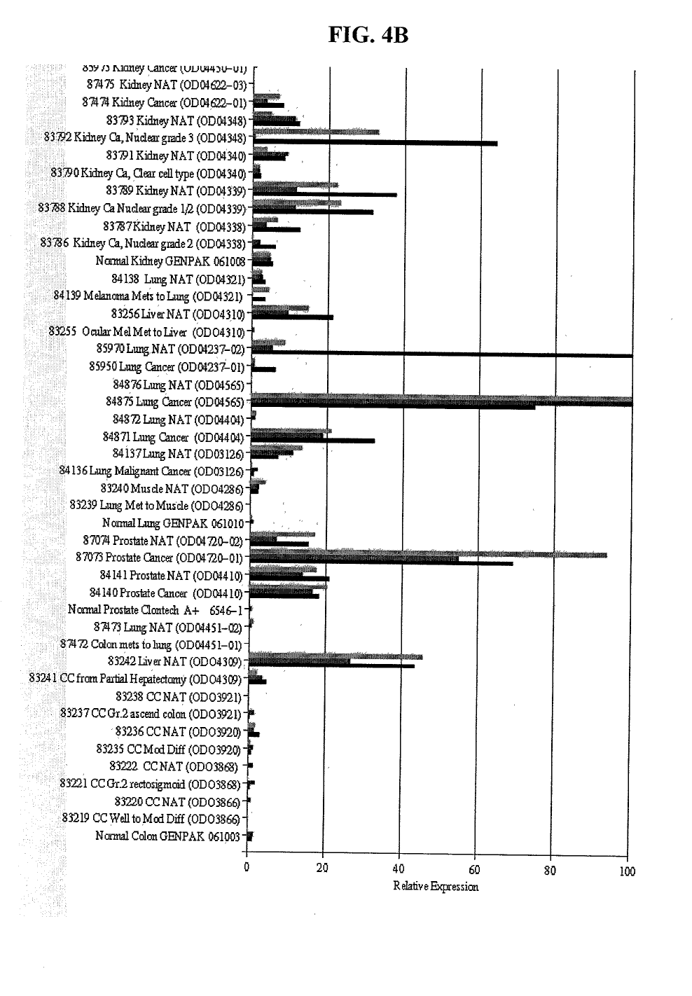

The relative expression results for clone 10129612 obtained using the primer-probe set Ag47 (Table 16) on a panel of cells drawn from various tissues are shown in FIG. 3. The comparable results found with a panel of tissues taken directly from surgical samples are shown in FIG. 4. In FIG. 4, tissues taken from surgically excised cancers are paired in many cases with immediately adjacent noncancerous tissue, designated “NAT” (for normal adjacent tissue). Triplicate runs are shown in FIG. 4. The results in FIG. 3 show that clone 10129612 in certain cancers, such as non-small cell lung cancer, an ovarian cancer cell line, and CNS cancers, but not at all or to a lesser extent in the corresponding normal cell lines. A similar finding is seen for a prostate cancer and a lung cancer, compared to the normal adjacent tissue, in FIG. 3. Therefore, clone 10129612 or the protein encoded by it can be used to target therapies or imaging diagnostics to treat prostate and lung cancer.

TABLE 16

| | | SEQ | |
|---|---|---|---|
| | | ID | Start |
| Primer/Probe | Sequence | NO | Position |
| Forward | 5′-CCAATGACCTGGCCACCA-3′ | 31 | |
| Probe | FAM-5′-CCAGAGTCCGTTCAGCTTCAGGACAGC-3′-TAMRA | 32 | |
| Reverse | 5′-GTGGCACGTTGCTGTTTAGC-3′ | 33 | |

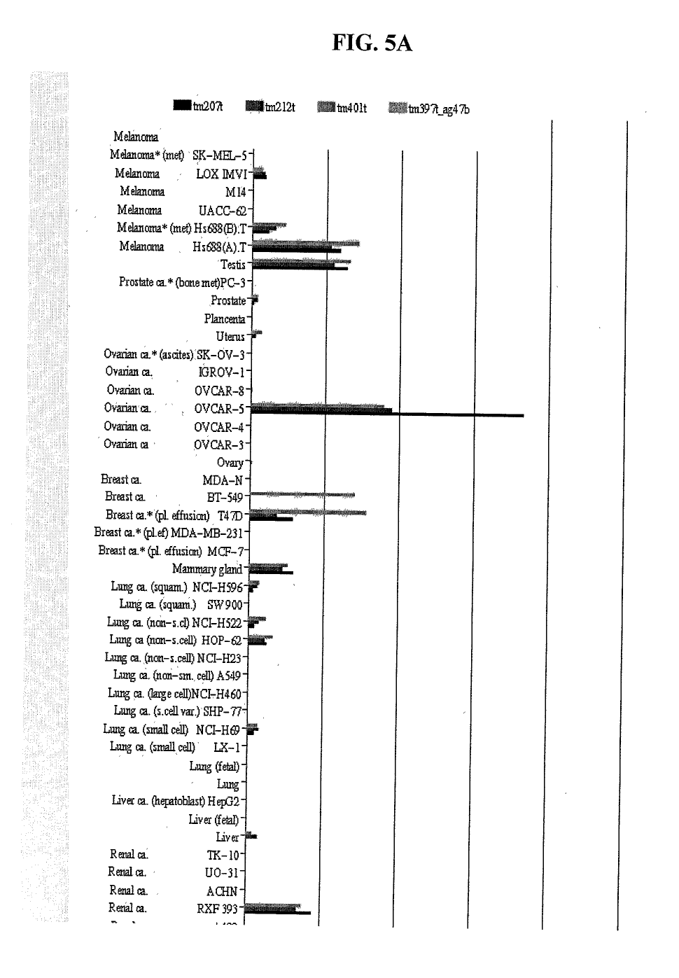

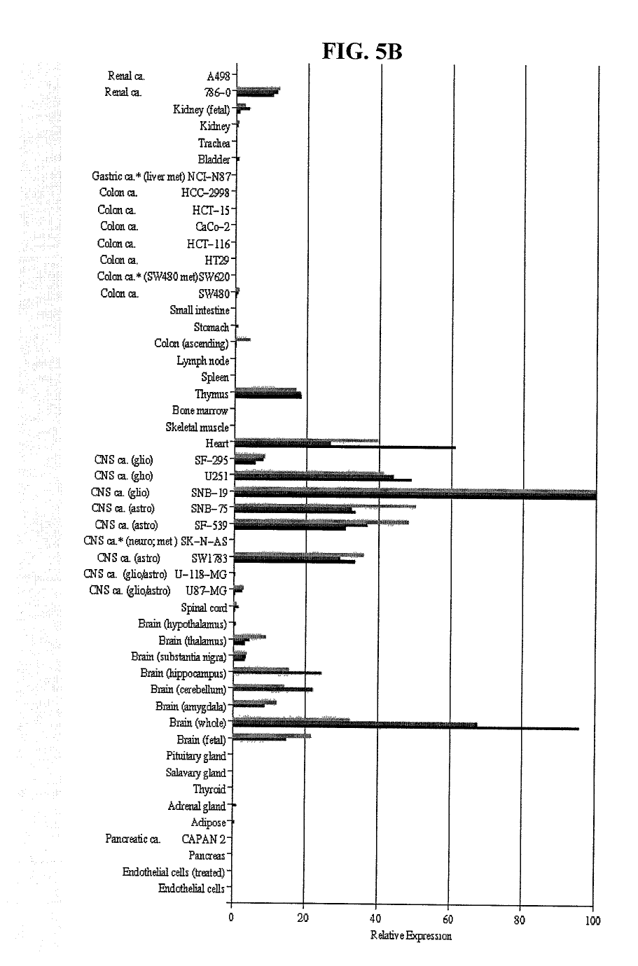

An additional expression analysis on clone 10129612 was carried out using a primer-probe set, Ag47b (Table 17), that targets a different portion of the gene. The results for triplicate runs are shown in FIG. 5. They confirm the differences identified in FIG. 3 for an ovarian cancer cell line, and for CNS cancers. They also show expression in a melanoma cell line, and a difference in kidney cancer vs. normal kidney.

TABLE 17

| | | SEQ ID | |
|---|---|---|---|
| Primer/Probe | Sequence | NO | Start Position |
| Forward | 5′-GAACGCCGGAGCATACAGA-3′ | 34 | 1065 |
| Probe | TET-5′-CCAGGTACTGCACAAACACGGCTTCAT-3′-TAMRA | 35 | |
| Reverse | 5′-GATGCCACAGGCCCACA-3′ | 36 | 1124 |

b) Clone 10168180.0.35, POLY5

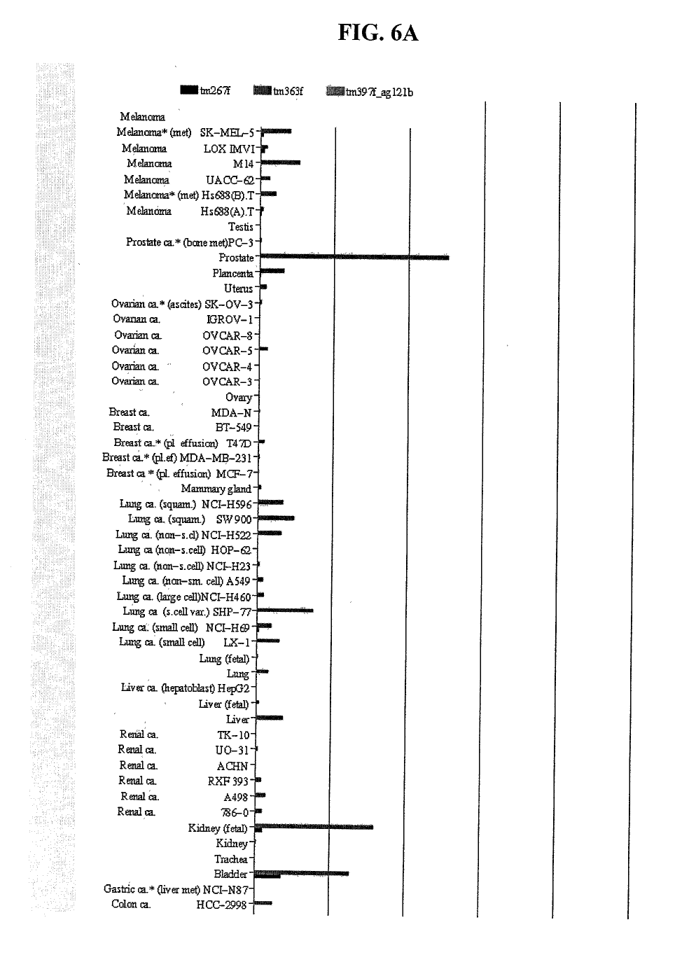

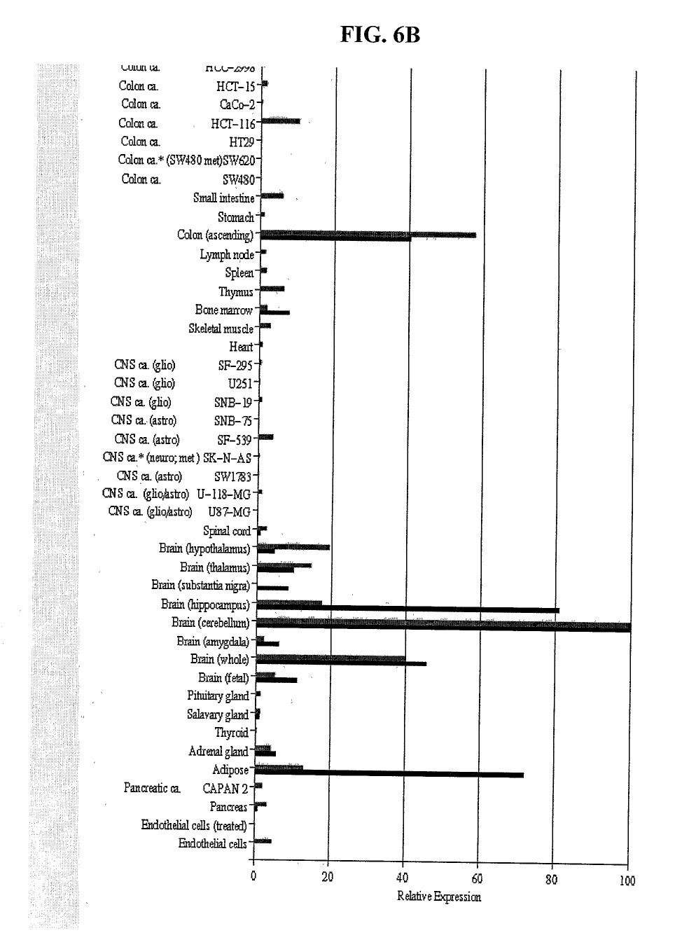

The tissue expression results for clone 10168180.0.35, obtained using primer-probe set Ag121 (Table 18) are shown in FIG. 6. It is seen that many cancer cell lines exhibit little or no expression of clone10268180.0.35 whereas the corresponding normal cell lines show high expression. Therefore, clone 10168180.0.35 or the protein encoded by it can be used to target therapies or imaging diagnostics to treat various forms of cancer.

TABLE 18

| | | SEQ ID | |
|---|---|---|---|
| Primer/Probe | Sequence | NO | Start Position |
| Forward | 5′-ACCAGGCTGGAGTGCAGTG-3′ | 37 | 419 |
| Probe | FAM-5′-CTCGATCTCAGCTCACTGCAACTGCC-3′-TAMRA | 38 | 439 |
| Reverse | 5′-AGGCAGGAGAATCGCTTGAA-3′ | 39 | 475 |

c) Clone 10354784.0.148, POLY6

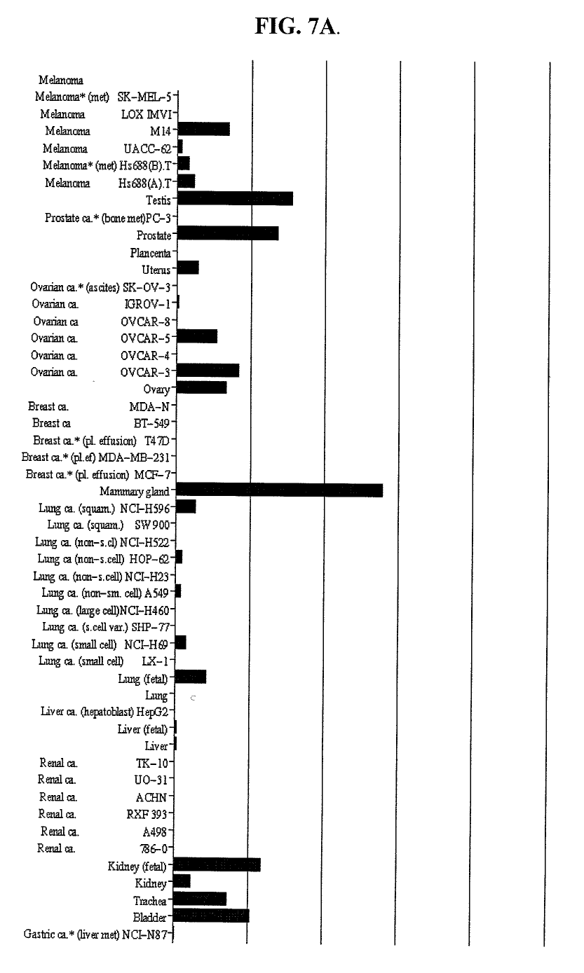

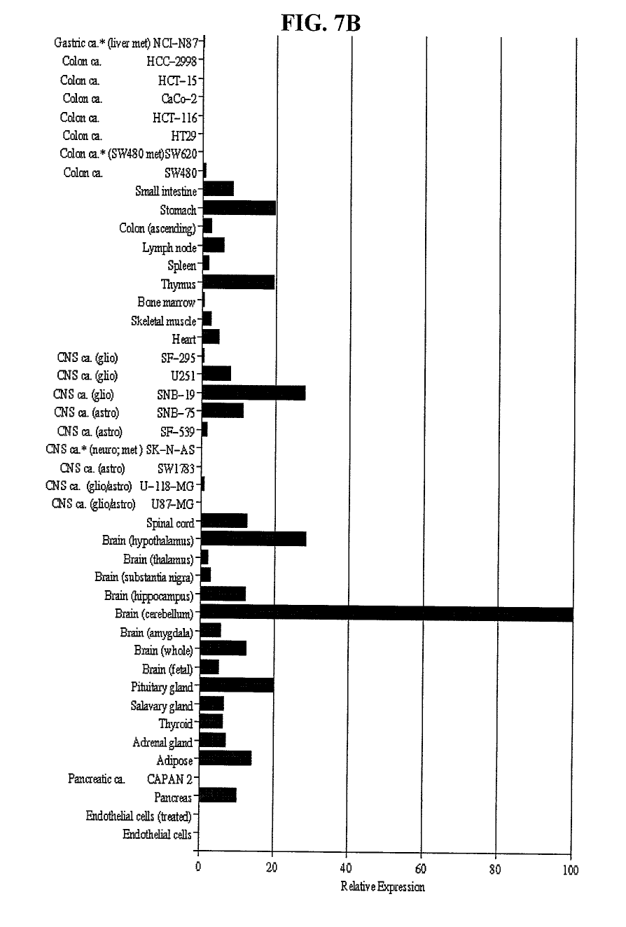

The tissue expression results for clone 10354784.0.148 obtained using the primer-probe set Ag91 (Table 19) are shown in FIG. 7. They indicate that this clone is expressed in many normal cell lines, but not in most cancers.

TABLE 19

| | | SEQ | |
|---|---|---|---|
| | | ID | |
| Primer/Probe | Sequence | NO | Start Position |
| Forward | 5′-TCTGTAGCACGCCCCACTCTA-3′ | 40 | 639 |
| Probe | TET-5′-GATCAAACAGCCATTCCGGGTCTTTCA-3′-TAMRA | 41 | |
| Reverse | 5′-GCAGTCCCAGAGAGCATGGA-3′ | 42 | 699 |

The tissue expression results obtained for clone 10354784.0.148 (also referred to as CG50670-02) using primer-probe set Ag1430 (Table 33) are shown in Table 34.

TABLE 33
Probe Name: Ag1430

| | | SEQ |
|---|---|---|
| Primer/ | | ID |
| Probe | Sequence | NO. |
| Forward | 5′-GCGGTGTTTCACCACATAGTAG-3′ | 78 |
| Primer | | |
| Probe | TET-5′-AGGCCGCCACACCAGTTCATATG-3′-TAMRA | 79 |
| Reverse | 5′-CGCACCTCCAAGACAGACT-3′ | 80 |
| Primer | | |

TABLE 34

| | Panel | | Panel |
|---|---|---|---|
| Tissue Name | 1.3 | Tissue Name | 4D |
| Liver adenocarcinoma | 10.7 | 93768_Secondary Th1_anti-CD28/anti- | 0.0 |
| | | CD3 | |
| Heart (fetal) | 6.2 | 93769_Secondary Th2_anti-CD28/anti- | 0.3 |
| | | CD3 | |
| Pancreas | 2.6 | 93770_Secondary Tr1_anti-CD28/anti- | 0.0 |
| | | CD3 | |
| Pancreatic ca.CAPAN 2 | 0.0 | 93573_Secondary Th1_resting day 4-6 | 0.0 |
| | | in IL-2 | |
| Adrenal gland | 11.9 | 93572_Secondary Th2_resting day 4-6 | 0.0 |
| | | in IL-2 | |
| Thyroid | 25.4 | 93571_Secondary Tr1_resting day 4-6 | 0.0 |
| | | in IL-2 | |
| Salivary gland | 12.1 | 93568_primary Th1_anti-CD28/anti- | 2.1 |
| | | CD3 | |
| Pituitary gland | 28.7 | 93569_primary Th2_anti-CD28/anti- | 0.0 |
| | | CD3 | |
| Brain (fetal) | 6.1 | 93570_primary Tr1_anti-CD28/anti- | 0.0 |
| | | CD3 | |
| Brain (whole) | 4.9 | 93565_primary Th1_resting dy 4-6 in | 0.0 |
| | | IL-2 | |
| Brain (amygdala) | 14.8 | 93566_primary Th2_resting dy 4-6 in | 0.0 |
| | | IL-2 | |
| Brain (cerebellum) | 5.0 | 93567_primary Tr1_resting dy 4-6 in | 0.0 |
| | | IL-2 | |
| Brain (hippocampus) | 51.4 | 93351_CD45RA CD4 lymphocyte_anti- | 8.6 |
| | | CD28/anti-CD3 | |
| Brain (thalamus) | 8.0 | 93352_CD45RO CD4 lymphocyte_anti- | 0.1 |
| | | CD28/anti-CD3 | |
| Cerebral Cortex | 6.3 | 93251_CD8 Lymphocytes_anti- | 0.0 |
| | | CD28/anti-CD3 | |
| Spinal cord | 14.8 | 93353_chronic CD8 Lymphocytes | 0.0 |
| | | 2ry_resting dy 4-6 in IL-2 | |
| CNS ca. (glio/astro) U87- | 0.8 | 93574_chronic CD8 Lymphocytes | 0.0 |
| MG | | 2ry_activated CD3/CD28 | |
| CNS ca. (glio/astro) U- | 5.7 | 93354_CD4_none | 0.0 |
| 118-MG | | | |
| CNS ca. (astro) SW1783 | 0.3 | 93252_Secondary Th1/Th2/Tr1_anti- | 0.0 |
| | | CD95 CH11 | |
| CNS ca.* (neuro; met) | 0.8 | 93103_LAK cells_resting | 0.0 |
| SK-N-AS | | | |
| CNS ca. (astro) SF-539 | 4.8 | 93788_LAK cells_IL-2 | 0.2 |
| CNS ca. (astro)SNB-75 | 14.8 | 93787_LAK cells_IL-2 + IL-12 | 0.0 |
| CNS ca. (glio) SNB-19 | 25.0 | 93789_LAK cells_IL-2 + IFN gamma | 0.0 |
| CNS ca. (glio)U251 | 19.1 | 93790_LAK cells_IL-2 + IL-18 | 0.0 |
| CNS ca. (glio)SF-295 | 4.7 | 93104_LAK cells_PMA/ionomycin and | 0.0 |
| | | IL-18 | |
| Heart | 3.2 | 93578_NK Cells IL-2_resting | 0.0 |
| Skeletal muscle | 4.0 | 93109_Mixed Lymphocyte | 0.0 |
| | | Reaction_Two Way MLR | |
| Bone marrow | 4.9 | 93110_Mixed Lymphocyte | 0.0 |
| | | Reaction_Two Way MLR | |
| Thymus | 11.5 | 93111_Mixed Lymphocyte | 0.0 |
| | | Reaction_Two Way MLR | |
| Spleen | 10.7 | 93112_Mononuclear Cells | 0.0 |
| | | (PBMCs)_resting | |
| Lymph node | 4.9 | 93113_Mononuclear Cells | 0.5 |
| | | (PBMCs)_PWM | |
| Colorectal | 6.3 | 93114_Mononuclear Cells | 0.3 |
| | | (PBMCs)_PHA-L | |
| Stomach | 17.4 | 93249_Ramos (B cell)_none | 0.0 |
| Small intestine | 44.1 | 93250_Ramos (B cell)_ionomycin | 0.0 |
| Colon ca. SW480 | 5.4 | 93349_B lymphocytes_PWM | 0.0 |
| Colon ca.* (SW480 | 0.2 | 93350_B lymphoytes_CD40L and IL-4 | 0.0 |
| met)SW620 | | | |
| Colon ca. HT29 | 0.0 | 92665_EOL-1 (Eosinophil)_dbcAMP | 0.0 |
| | | differentiated | |
| Colon ca. HCT-116 | 0.6 | 93248_EOL-1 | 3.9 |
| | | (Eosinophil)_dbcAMP/PMAionomycin | |
| Colon ca.CaCo-2 | 0.0 | 93356_Dendritic Cells none | 0.0 |
| 83219 CC Well to Mod | | | |
| Diff (ODO3866) | 13.4 | 93355_Dendritic Cells_LPS 100 ng/ml | 0.0 |
| Colon ca. HCC-2998 | 0.0 | 93775_Dendritic Cells_anti-CD40 | 0.0 |
| Gastric ca.* (liver met) | 1.9 | 93774_Monocytes_resting | 0.0 |
| NCI-N87 | | | |
| Bladder | 4.3 | 93776_Monocytes_LPS 50 ng/ml | 0.0 |
| Trachea | 66.0 | 93581_Macrophages_resting | 0.0 |
| Kidney | 4.2 | 93582_Macrophages_LPS 100 ng/ml | 0.0 |
| Kidney (fetal) | 21.9 | 93098_HUVEC (Endothelial)_none | 0.0 |
| Renal ca.786-0 | 0.2 | 93099_HUVEC (Endothelial)_starved | 0.2 |
| Renal ca.A498 | 10.5 | 93100_HUVEC (Endothelial)_IL-1b | 0.0 |
| Renal ca.RXF 393 | 1.4 | 93779_HUVEC (Endothelial)_IFN | 0.0 |
| | | gamma | |
| Renal ca.ACHN | 0.5 | 93102_HUVEC (Endothelial)_TNF | 0.0 |
| | | alpha + IFN gamma | |
| Renal ca.UO-31 | 0.4 | 93101_HUVEC (Endothelial)_TNF | 0.0 |
| | | alpha + IL4 | |
| Renal ca.TK-10 | 0.0 | 93781_HUVEC (Endothelial)_IL-11 | 0.0 |
| Liver | 0.1 | 93583_Lung Microvascular Endothelial | 0.0 |
| | | Cells_none | |
| Liver (fetal) | 3.2 | 93584_Lung Microvascular Endothelial | 0.0 |
| | | Cells_TNFa (4 ng/ml) and IL1b (1 ng/ml) | |
| Liver ca. (hepatoblast) | 0.0 | 92662_Microvascular Dermal | 0.0 |
| HepG2 | | endothelium_none | |
| Lung | 8.7 | 92663_Microsvasular Dermal | 0.0 |
| | | endothelium_TNFa (4 ng/ml) and IL1b | |
| | | (1 ng/ml) | |
| Lung (fetal) | 20.3 | 93773_Bronchial epithelium_TNFa (4 ng/ml) | 0.2 |
| | | and IL1b (1 ng/ml)** | |
| Lung ca. (small cell)LX-1 | 0.0 | 93347_Small Airway Epithelium_none | 0.0 |
| Lung ca. (small cell) NCI- | 3.7 | 93348_Small Airway Epithelium_TNFa | 0.2 |
| H69 | | (4 ng/ml) and IL1b (1 ng/ml) | |
| Lung ca. (s.cell var.) SHP- | 0.7 | 92668_Coronery Artery SMC_resting | 0.4 |
| 77 | | | |
| Lung ca. (large cell)NCI- | 0.2 | 92669_Coronery Artery SMC_TNFa (4 ng/ml) | 0.0 |
| H460 | | and IL1b (1 ng/ml) | |
| Lung ca. (non-sm. cell) | 1.8 | 93107_astrocytes_resting | 8.4 |
| A549 | | | |
| Lung ca. (non-s.cell) NCI- | 0.2 | 93108_astrocytes_TNFa (4 ng/ml) and | 1.9 |
| H23 | | IL1b (1 ng/ml) | |
| Lung ca (non-s.cell) | 4.4 | 92666_KU-812 (Basophil)_resting | 0.0 |
| HOP-62 | | | |
| Lung ca. (non-s.cl) NCI- | 0.4 | 92667_KU-812 | 0.0 |
| H522 | | (Basophil)_PMA/ionoycin | |
| Lung ca. (squam.)SW 900 | 0.5 | 93579_CCD1106 (Keratinocytes)_none | 2.3 |
| Lung ca. (squam.) NCI- | 1.5 | 93580_CCD1106 | 1.6 |
| H596 | | (Keratinocytes)_TNFa and IFNg** | |
| Mammary gland | 100.0 | 93791_Liver Cirrhosis | 3.5 |
| Breast ca.* (pl. effusion) | 0.1 | 93792_Lupus Kidney | 1.6 |
| MCF-7 | | | |
| Breast ca.* (pl.ef) MDA- | 0.4 | 93577_NCI-H292 | 0.2 |
| MB-231 | | | |
| Breast ca.* (pl. | 0.1 | 93358_NCI-H292_IL-4 | 0.2 |
| effusion)T47D | | | |
| Breast ca. BT-549 | 3.3 | 93360_NCI-H292_IL-9 | 0.0 |
| Breast ca. MDA-N | 0.0 | 93359_NCI-H292_IL-13 | 0.0 |
| Ovary | 98.6 | 93357_NCI-H292_IFN gamma | 0.2 |
| Ovarian ca. OVCAR-3 | 32.5 | 93777_HPAEC_- | 0.5 |
| Ovarian ca. OVCAR-4 | 0.3 | 93778_HPAEC_IL-1 beta/TNA alpha | 0.0 |
| Ovarian ca.OVCAR-5 | 11.7 | 93254_Normal Human Lung | 1.4 |
| | | Fibroblast_none | |
| Ovarian ca. OVCAR-8 | 1.0 | 93253_Normal Human Lung | 3.2 |
| | | Fibroblast_TNFa (4 ng/ml) and IL-1b (1 ng/ml) | |
| Ovarian ca.IGROV- 1 | 1.3 | 93257_Normal Human Lung | 3.1 |
| | | Fibroblast_IL-4 | |
| Ovarian ca.* (ascites) SK- | 0.9 | 93256_Normal Human Lung | 1.5 |
| OV-3 | | Fibroblast_IL-9 | |
| Uterus | 66.0 | 93255_Normal Human Lung | 3.3 |
| | | Fibroblast_IL-13 | |
| Plancenta | 4.6 | 93258_Normal Human Lung | 9.5 |
| | | Fibroblast_IFN gamma | |
| Prostate | 19.9 | 93106_Dermal Fibroblasts | 25.7 |
| | | CCD1070_resting | |
| Prostate ca.* (bone | 6.4 | 93361_Dermal Fibroblasts | 10.9 |
| met)PC-3 | | CCD1070_TNF alpha 4 ng/ml | |
| Testis | 15.3 | 93105_Dermal Fibroblasts | 18.3 |
| | | CCD1070_IL-1 beta 1 ng/ml | |
| Melanoma Hs688(A).T | 5.7 | 93772_dermal fibroblast_IFN gamma | 100.0 |
| Melanoma* (met) | 1.4 | 93771_dermal fibroblast_IL-4 | 29.1 |
| Hs688(B).T | | | |
| MelanomaUACC-62 | 0.5 | 93259_IBD Colitis 1** | 2.1 |
| Melanoma M14 | 8.0 | 93260_IBD Colitis 2 | 2.0 |
| Melanoma LOX IMVI | 0.0 | 93261_IBD Crohns | 4.3 |
| Melanoma* (met) SK- | 0.6 | 735010_Colon_normal | 36.1 |
| MEL-5 | | | |
| Adipose | 36.9 | 735019_Lung_none | 12.9 |
| | | 64028-1_Thymus_none | 10.6 |
| | | 64030-1_Kidney_none | 24.2 |

Panel 1.3: This panel is comprised of both normal tissues and tumor tissues. Normal tissues include lung ovary, testes, prostate, placenta, mammary gland kidney, trachea bladder small intestine stomach, colon, lymph node, heart, spleen, thymus, bone marrow skeletal muscle, brain, adipose, pancreas, salivary gland thyroid and adrenals. POLY6 transcript was detected in the brain (hippocampus), mammary gland, ovary, uterus, trachea and small intestine.

Panel 4D: POLY6 transcript is highly up regulated (>4 fold) in dermal fibroblasts after treatment with gamma interferon as compared with untreated fibroblasts as well as fibroblasts treated with Il-4, Il-1 or TNF alpha. Lung fibroblasts also up regulate this molecule in response to gamma interferon. Normal lung, kidney and colon express this transcript.

Potential Role(s) of POLY6 in Inflammation: The restricted expression of this transcript to IFN gamma treated fibroblasts indicates it may be induced by activated Th1 or Tc1 cells. Both of these cell types produce gamma interferon and are involved in acute and chronic inflammatory diseases such as delayed type hypersensitivity, psoriasis, viral infections and perhaps emphysema. Therefore, expression of POLY6 may potentiate the inflammatory response.

Impact of Therapeutic Targeting of POLY6: Based on the expression profile of this transcript and the types of cytokines which induce it, antibodies to POLY6 can be used to inhibit inflammation and subsequent tissue damage in the skin due to delayed type hypersensitivity responses and psoriasis as well as in lung due to viral infection and emphysema.

d) Clone 16532807.0.137, POLY8

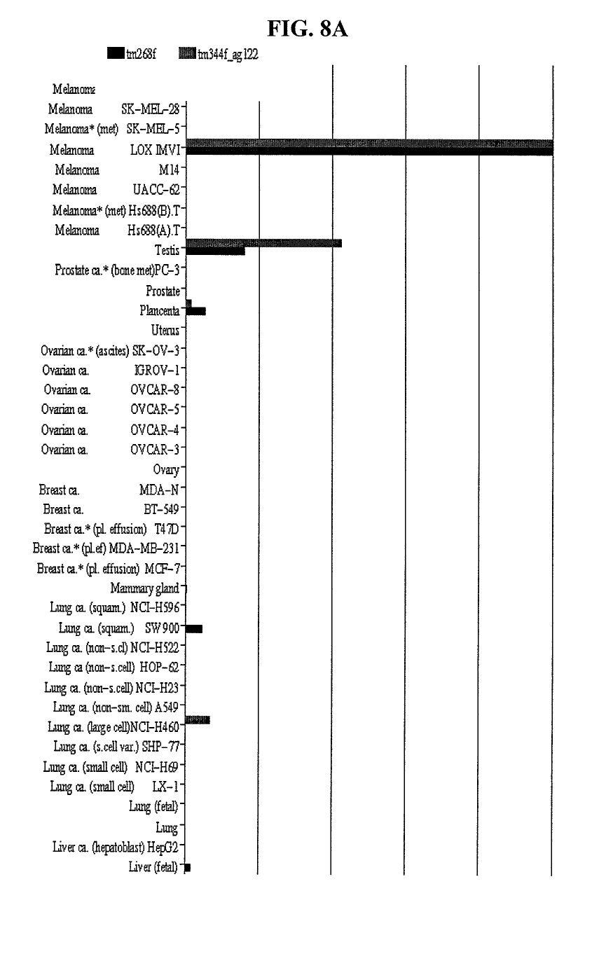

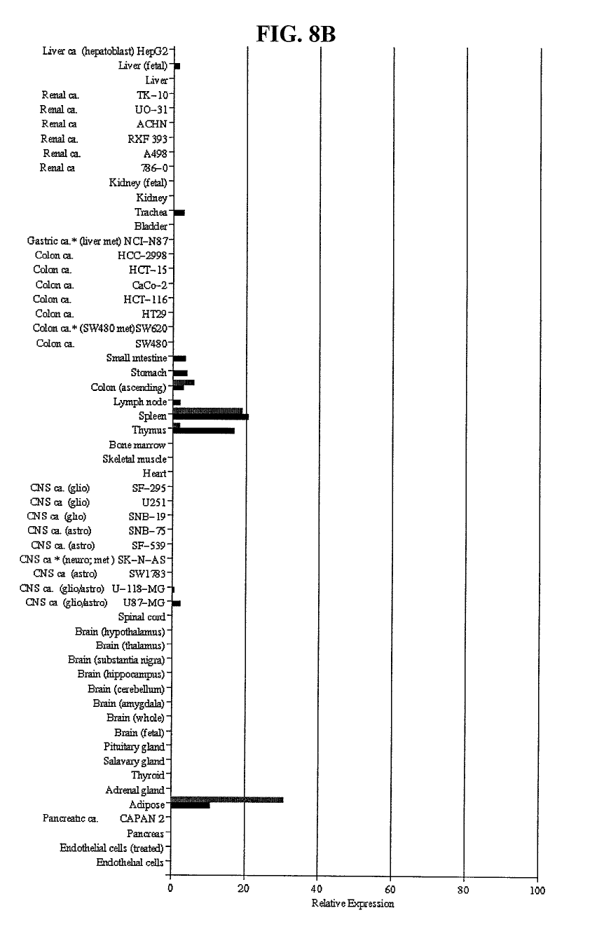

The tissue expression analysis obtained for clone 16532807.0.137 using the primers and probe of Table 20 (Ag122) is shown in FIG. 8. The analysis was run in duplicate. This gene is selectively expressed in only a few normal cell lines, but is highly overexpressed in melanoma LOX IMVI. It is also expressed in certain lung cancer cells.

From the results of the real-time quantitative PCR analysis, it is concluded that clone 16532807.0.137 is highly expressed in certain melanomas and lung cancer tissues. Therefore, clone 16532807.0.137 or the protein encoded by it can be used to target therapies or imaging diagnostics to treat melanoma and lung cancer.

TABLE 29

| | | SEQ | |
|---|---|---|---|
| | | ID | |
| Primer/Probe | Sequence | NO | Start Position |
| Forward | 5′-TTCCATGCCTCGCAAATGTAT-3′ | 43 | 870 |
| Probe | FAM-5′-CAAAGCACTGCCCTCTGGAACTGCA-3′-TAMRA | 44 | 829 |
| Reverse | 5′-GCCGTTTGTCCTCTAAGCAGA-3′ | 45 | 807 |

e) Clone 17941787.0.3, POLY10

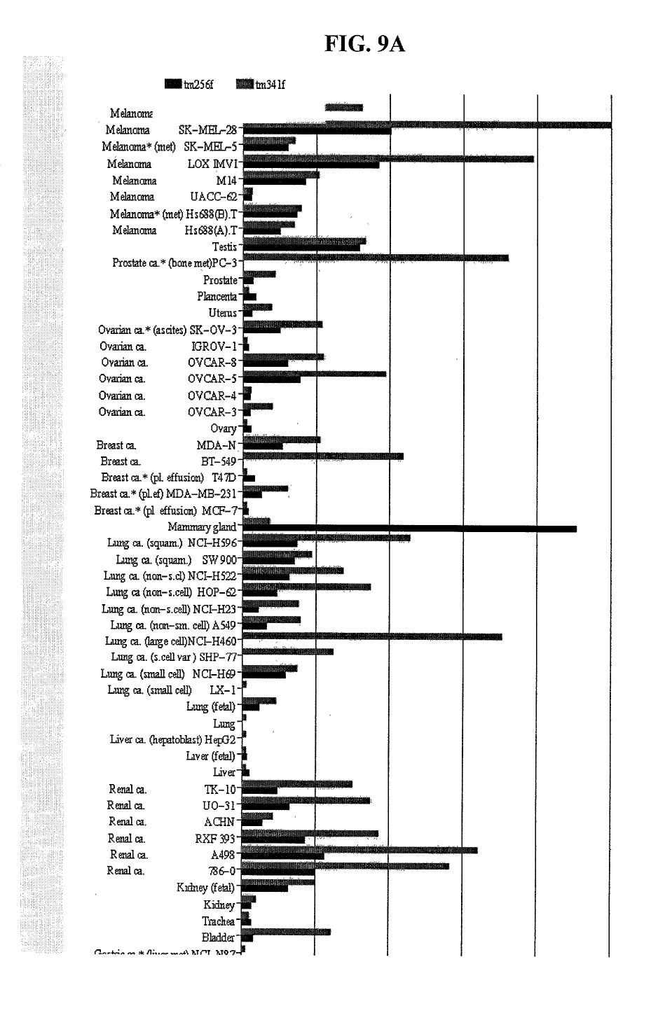

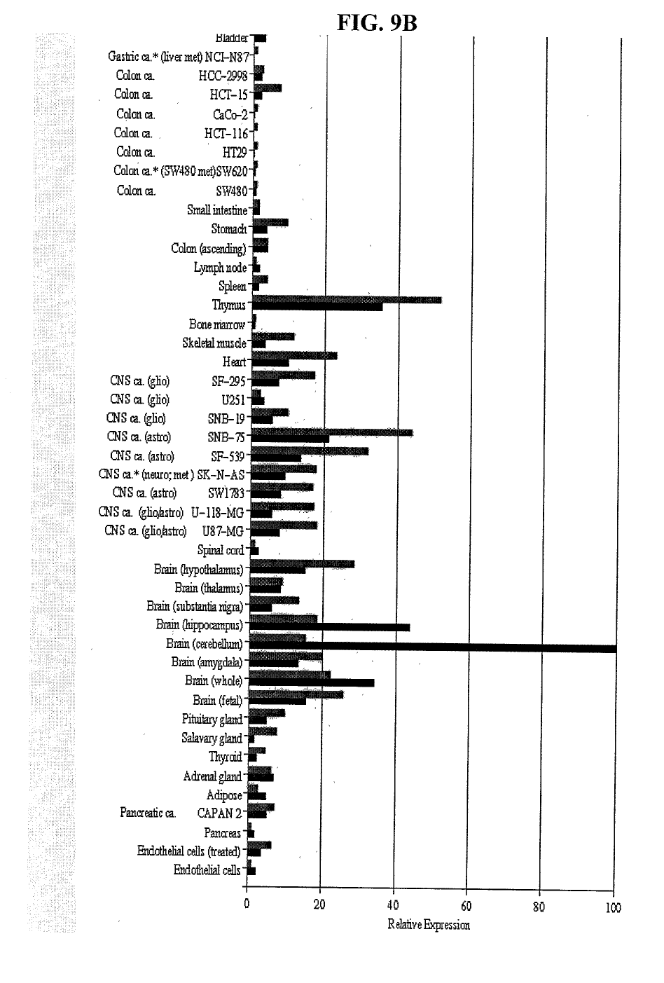

The tissue expression analysis for clone 17941787.0.3, obtained using the primer-probe set Ag96 (Table 21), is shown in FIG. 9. The results show that this clone is broadly expressed in many cell lines. It is highly expressed in melanoma, and strongly expressed differentially in prostate cancer metastasis, ovarian cancer, lung cancer cells and renal cancer cells compared to the corresponding normal cell lines.

From the results of the real-time quantitative PCR analysis, it is concluded that clone 17941787.0.3 could be useful in distinguishing melanoma, prostate cancer, ovarian cancer, lung cancer, renal cancer and pancreas cancer from normal prostate, ovary, lung, kidney and pancreas tissues. Its high expression in melanoma, prostate cancer, ovarian cancer, lung cancer, renal cancer and pancreas cancer suggests that clone 17941787.0.3 can be used to target therapies or imaging diagnostics to treat these diseases.

TABLE 21

| | | SEQ ID | |
|---|---|---|---|
| Primer/Probe | Sequence | NO | Start Position |
| Forward | 5′-CCAAGTAGATGGGTTCTGTTTGC-3′ | 46 | 1169 |
| Probe | FAM-5′-CCCAGTTACCTCCACAGGGTATTTCCCA-3′-TAMRA | 47 | 1194 |
| Reverse | 5′-CGACGCTGCTGCTCAGTATAAC-3′ | 48 | 1282 |

f) Clone 21636818.0.57, POLY12

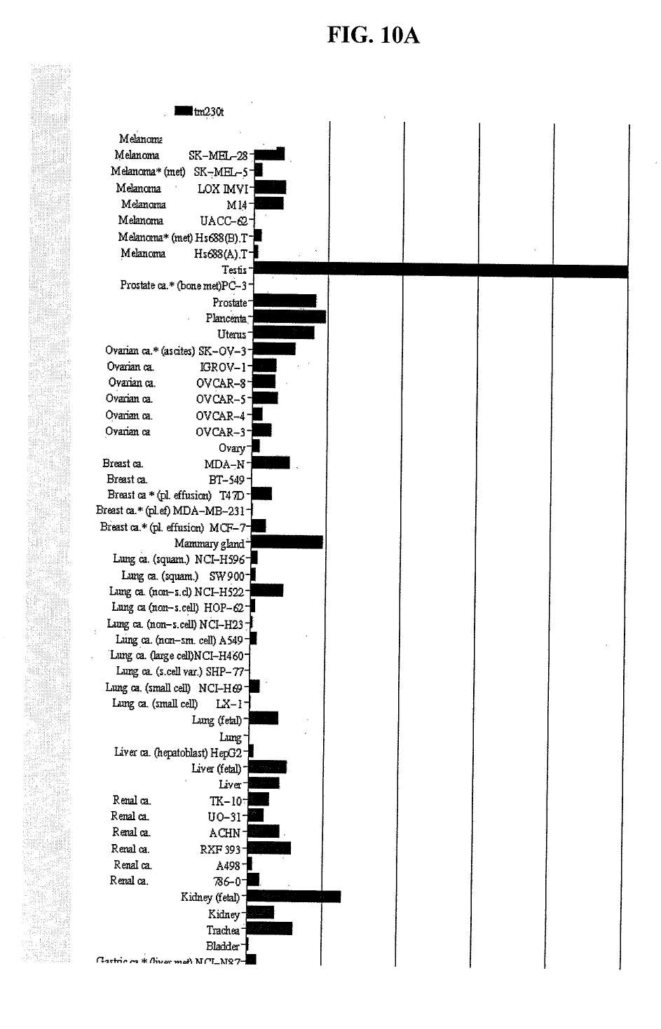

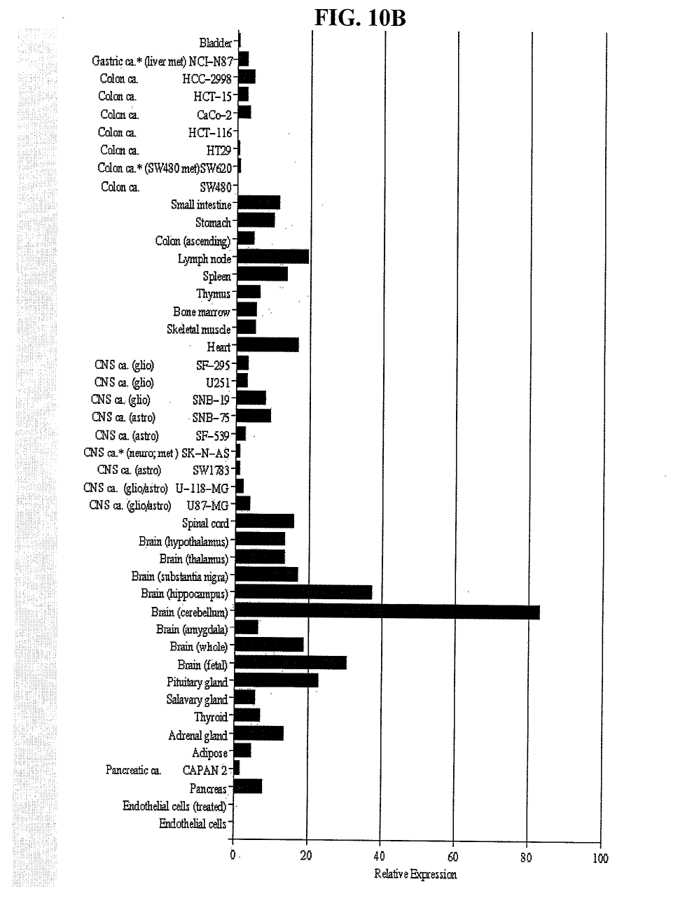

The expression analysis results obtained for clone 21636818.0.57 using primer-probe set ag59 (table 22) are shown in FIG. 10. This clone is broadly expressed in many cell lines.

From the results of the real-time quantitative PCR analysis it is concluded that clone 21636818.0.57 is highly expressed in a variety of melanoma, ovarian cancer and lung cancer tissues. Therefore, clone 21636818.0.57 or the protein encoded by it can be used to target therapies or imaging diagnostics to treat melanoma, ovarian cancer and lung cancer. Interestingly, the expression of clone 21636818.0.57 is much lower in prostate cancer than normal prostate, suggesting that clone 21636818.0.57 could be used as a marker for the development of prostate cancer.

TABLE 22

| | | SEQ | |
|---|---|---|---|
| | | ID | Start |
| Primer/Probe | Sequence | NO | Position |
| Forward | 5′-TCTGCCCCGCGTCTGTAC-3′ | 49 | 559 |
| Probe | TET-5′-TGGTTTCTCTCTGTGCTCTCGTAACACCTCAG-3′-TAMRA | 50 | 526 |
| Reverse | 5′-CTCCACCACACGGAATTACCTT-3′ | 51 | 501 |

g) Clone 13043743.0.15, POLY7

The expression analysis results obtained for clone 13043743.0.15 using primer-probe set Ag46 (Table 23).

TABLE 23

| | | SEQ ID | Start |
|---|---|---|---|
| Primer/Probe | Sequence | NO | Position |
| Forward Primer | 5′-CCTGCCATGTTTGGACTGGT-3′ | 52 | 494 |
| Probe | FAM-5′-TTTTGGCCCATCACTTGGGCTCATTC-3′-TAMRA | 53 | 520 |
| Reverse Primer | 5′-GCAACCTAAGGCATGACTGTTG-3′ | 54 | 548 |

The results of this analysis are shown in Table 24. The higher the relative expression value corresponding to a specific sample, the greater the intensity of the expression of the gene in that sample. From these results, it is seen that the gene is specifically expressed in the thalamus of the brain at a measurably higher level than placenta, prostate, ovary, lung, liver, heart, lymphoid tissues, thyroid, adipose, pancreas and other regions of the brain.

TABLE 24

| Tissue_Name/Run_Name | 1tm506f_ag46 |
|---|---|
| Endothelial cells | 0.44 |
| Endothelial cells (treated) | 0.21 |
| Pancreas | 0.05 |
| Pancreatic ca. CAPAN 2 | 0.21 |
| Adipose | 0 |
| Adrenal gland | 0.02 |
| Thyroid | 0.05 |
| Salavary gland | 0.05 |
| Pituitary gland | 0 |
| Brain (fetal) | 0.04 |
| Brain (whole) | 0.02 |
| Brain (amygdala) | 3.76 |
| Brain (cerebellum) | 0.01 |
| Brain (hippocampus) | 0.11 |
| Brain (substantia nigra) | 0.49 |
| Brain (thalamus) | 100 |
| Brain (hypothalamus) | 0.04 |
| Spinal cord | 0.04 |
| CNS ca. (glio/astro) U87-MG | 0.16 |
| CNS ca. (glio/astro) U-118-MG | 0.21 |
| CNS ca. (astro) SW1783 | 0.27 |
| CNS ca.* (neuro; met) SK-N-AS | 0.21 |
| CNS ca. (astro) SF-539 | 0.21 |
| CNS ca. (astro) SNB-75 | 0.16 |
| CNS ca. (glio) SNB-19 | 0 |
| CNS ca. (glio) U251 | 0.16 |
| CNS ca. (glio) SF-295 | 0.12 |
| Heart | 0.07 |
| Skeletal muscle | 0.12 |
| Bone marrow | 0 |
| Thymus | 0.03 |
| Spleen | 0.07 |
| Lymph node | 0.05 |
| Colon (ascending) | 0.03 |
| Stomach | 0.04 |
| Small intestine | 0.05 |
| Colon ca. SW480 | 0.35 |
| Colon ca.* (SW480 met) SW620 | 0.35 |
| Colon ca. HT29 | 0.27 |
| Colon ca. HCT-116 | 0.57 |
| Colon ca. CaCo-2 | 0.12 |
| Colon ca. HCT-15 | 0 |
| Colon ca. HCC-2998 | 0.16 |
| Gastric ca.* (liver met) NCI-N87 | 0.07 |
| Bladder | 0.05 |
| Trachea | 0.04 |
| Kidney | 0.05 |
| Kidney (fetal) | 0.02 |
| Renal ca. 786-0 | 0.16 |
| Renal ca. A498 | 0.16 |
| Renal ca. RXF 393 | 0.21 |
| Renal ca. ACHN | 0.27 |
| Renal ca. UO-31 | 0.16 |
| Renal ca. TK-10 | 0.16 |
| Liver | 0.05 |
| Liver (fetal) | 0 |
| Liver ca. (hepatoblast) HepG2 | 0.21 |
| Lung | 0 |
| Lung (fetal) | 0.05 |
| Lung ca. (small cell) LX-1 | 0.21 |
| Lung ca. (small cell) NCI-H69 | 0.07 |
| Lung ca. (s.cell var.) SHP-77 | 0.27 |
| Lung ca. (large cell) NCI-H460 | 0.35 |
| Lung ca. (non-sm. cell) A549 | 0.12 |
| Lung ca. (non-s.cell) NCI-H23 | 0.16 |
| Lung ca (non-s.cell) HOP-62 | 0.16 |
| Lung ca. (non-s.cl) NCI-H522 | 0.16 |
| Lung ca. (squam.) SW 900 | 0.72 |
| Lung ca. (squam.) NCI-H596 | 0.07 |
| Mammary gland | 0.02 |
| Breast ca.* (pl. effusion) MCF-7 | 0.12 |
| Breast ca.* (pl.ef) MDA-MB-231 | 0.27 |
| Breast ca.* (pl. effusion) T47D | 0.05 |
| Breast ca. BT-549 | 0.44 |
| Breast ca. MDA-N | 0.12 |
| Ovary | 0.05 |
| Ovarian ca. OVCAR-3 | 0.16 |
| Ovarian ca. OVCAR-4 | 0.21 |
| Ovarian ca. OVCAR-5 | 0.05 |
| Ovarian ca. OVCAR-8 | 0 |
| Ovarian ca. IGROV-1 | 0.16 |
| Ovarian ca.* (ascites) SK-OV-3 | 0.16 |
| Uterus | 0.04 |
| Plancenta | 0.04 |
| Prostate | 0.03 |
| Prostate ca.* (bone met)PC-3 | 0.35 |
| Testis | 0 |
| Melanoma Hs688(A).T | 0.21 |
| Melanoma* (met) Hs688(B).T | 0.16 |
| Melanoma UACC-62 | 0.35 |
| Melanoma M14 | 0.12 |
| Melanoma LOX IMVI | 0.12 |
| Melanoma* (met) SK-MEL-5 | 0.21 |
| Melanoma SK-MEL-28 | 0.12 |

From the results of the real-time quantitative PCR analysis it is concluded that clone 13043743.0.15 is specifically expressed in the thalamus of the brain and can be used to target therapies or imaging diagnostics to treat any diseases or pathologies associated with brain thalamus.

h) Clone 20936375.0.104, POLY11

The expression analysis results obtained for clone 20936375.0.104 using primer-probe set Ag174 (Table 25) are shown in Table 26.

TABLE 25

| | | SEQ ID | Start |
|---|---|---|---|
| Primer/Probe | Sequence | NO | Position |
| Forward Primer | 5′-AGGACATAGGATGCAACACTTGAG-3′ | 55 | 798 |
| Probe | TET-5′-ACCTGCCGGCCCTTGGTTCCT-3′-TAMRA | 56 | 774 |
| Reverse Primer | 5′-CCAGCGCTCCCCATCAC-3′ | 57 | 746 |

The results of this analysis are shown in Table 26. The higher the relative expression value corresponding to a specific sample, the greater the intensity of the expression of the gene in that sample. From these results, it is seen that the gene is specifically expressed in melanoma, prostate cancer, ovarian cancer, breast cancer, lung cancer, liver cancer, renal cancer, colon cancer and pancreas cancer at a measurably higher level than the corresponding normal prostate, ovary, mammary gland, lung, liver, kidney, colon and pancreas tissues.

TABLE 26

| Tissue_Name/Run_Name | tm561t_ag174 |
|---|---|
| Endothelial cells | 16.1 |
| Endothelial cells (treated) | 10.33 |
| Pancreas | 6.57 |
| Pancreatic ca. CAPAN 2 | 40.75 |
| Adipose | 2.49 |
| Adrenal gland | 17.36 |
| Thyroid | 12.42 |
| Salavary gland | 9.91 |
| Pituitary gland | 6.4 |
| Brain (fetal) | 8.23 |
| Brain (whole) | 6.9 |
| Brain (amygdala) | 15.54 |
| Brain (cerebellum) | 8.4 |
| Brain (hippocampus) | 15.83 |
| Brain (substantia nigra) | 18.83 |
| Brain (thalamus) | 19.38 |
| Brain (hypothalamus) | 12.9 |
| Spinal cord | 17.05 |
| CNS ca. (glio/astro) U87-MG | 25.5 |
| CNS ca. (glio/astro) U-118-MG | 12.98 |
| CNS ca. (astro) SW1783 | 16.46 |
| CNS ca.* (neuro; met) SK-N-AS | 93.25 |
| CNS ca. (astro) SF-539 | 14.85 |
| CNS ca. (astro) SNB-75 | 7.76 |
| CNS ca. (glio) SNB-19 | 19.82 |
| CNS ca. (glio) U251 | 7.4 |
| CNS ca. (glio) SF-295 | 7.53 |
| Heart | 49.59 |
| Skeletal muscle | 55.89 |
| Bone marrow | 10.77 |
| Thymus | 7.69 |
| Spleen | 11.97 |
| Lymph node | 4.65 |
| Colon (ascending) | 6.85 |
| Stomach | 4.61 |
| Small intestine | 10.12 |
| Colon ca. SW480 | 16.82 |
| Colon ca.* (SW480 met)SW620 | 20.37 |
| Colon ca. HT29 | 60.38 |
| Colon ca. HCT-116 | 53.14 |
| Colon ca. CaCo-2 | 35.77 |
| Colon ca. HCT-15 | 43.99 |
| Colon ca. HCC-2998 | 0 |
| Gastric ca.* (liver met) NCI-N87 | 28.32 |
| Bladder | 25.74 |
| Trachea | 4.74 |
| Kidney | 31.6 |
| Kidney (fetal) | 6.68 |
| Renal ca. 786-0 | 47.01 |
| Renal ca. A498 | 36.56 |
| Renal ca. RXF 393 | 15.88 |
| Renal ca. ACHN | 25.39 |
| Renal ca. UO-31 | 17.19 |
| Renal ca. TK-10 | 25.5 |
| Liver | 26.29 |
| Liver (fetal) | 17.14 |
| Liver ca. (hepatoblast) HepG2 | 74.18 |
| Lung | 4.31 |
| Lung (fetal) | 2.6 |
| Lung ca. (small cell) LX-1 | 46 |
| Lung ca. (small cell) NCI-H69 | 9.77 |
| Lung ca. (s.cell var.) SHP-77 | 16.46 |
| Lung ca. (large cell) NCI-H460 | 100 |
| Lung ca. (non-sm. cell) A549 | 52.54 |
| Lung ca. (non-s.cell) NCI-H23 | 26.74 |
| Lung ca. (non-s.cell) HOP-62 | 13.01 |
| Lung ca. (non-s.cl) NCI-H522 | 54.22 |
| Lung ca. (squam.) SW 900 | 37.74 |
| Lung ca. (squam.) NCI-H596 | 12.4 |
| Mammary gland | 7.37 |
| Breast ca.* (pl. effusion) MCF-7 | 40.48 |
| Breast ca.* (pl.ef) MDA-MB-231 | 25.22 |
| Breast ca.* (pl. effusion) T47D | 10.94 |
| Breast ca. BT-549 | 36.46 |
| Breast ca. MDA-N | 45.18 |
| Ovary | 2.31 |
| Ovarian ca. OVCAR-3 | 27.48 |
| Ovarian ca. OVCAR-4 | 22.09 |
| Ovarian ca. OVCAR-5 | 20.82 |
| Ovarian ca. OVCAR-8 | 27.65 |
| Ovarian ca. IGROV-1 | 36.56 |
| Ovarian ca.* (ascites) SK-OV-3 | 27.67 |
| Uterus | 20.98 |
| Plancenta | 7.88 |
| Prostate | 9.83 |
| Prostate ca.* (bone met)PC-3 | 80.42 |
| Testis | 7.54 |
| Melanoma Hs688(A).T | 14.46 |
| Melanoma* (met) Hs688(B).T | 12.92 |
| Melanoma UACC-62 | 19.07 |
| Melanoma M14 | 24.19 |
| Melanoma LOX IMVI | 6.7 |
| Melanoma* (met) SK-MEL-5 | 41.86 |
| Melanoma SK-MEL-28 | 8.82 |

From the results of the real-time quantitative PCR analysis, it is concluded that clone 20936375.0.104 could be useful in distinguishing melanoma, prostate cancer, ovarian cancer, breast cancer, lung cancer, liver cancer, renal cancer, colon cancer and pancreas cancer from normal prostate, ovary, mammary gland, lung, liver, kidney, colon and pancreas tissues. The high expression of 20936375.0.104 in melanoma, prostate cancer, ovarian cancer, breast cancer, lung cancer, liver cancer, renal cancer, colon cancer and pancreas cancer suggests that clone 20936375.0.104 can be used to target therapies or imaging diagnostics to treat these diseases.

i) Clone 23208248, POLY1

The expression analysis results obtained for clone 23208248 using primer-probe set Ag161 (Table 27) are shown in Table 28.

TABLE 27

| Primer/Probe | Sequence | SEQ ID NO. | Start Position |
|---|---|---|---|
| Forward Primer | 5′-AGTCGGAGCCCATTGACCTT-3′ | 58 | 1111 |
| Probe | TET-5′-CCCCTGCATTGCCTATGGGCTTG-3′-TAMRA | 59 | 1133 |
| Reverse Primer | 5′-ACCTTGTATGCTGAGGTCTCCTTG-3′ | 60 | 1163 |

The results of this analysis are shown in Table 28. The higher the relative expression value corresponding to a specific sample, the greater the intensity of the expression of the gene in that sample. From these results, it is seen that the gene is specifically expressed in mammary gland and certain melanoma and breast cancer tissues at a measurably higher level than placenta, prostate, ovary, lung, liver, heart, lymphoid tissues, thyroid, adipose and pancreas.

TABLE 28

| Tissue_Name/Run_Name | tm342t | tm295t |
|---|---|---|
| Endothelial cells | 0.07 | 26.54 |
| Endothelial cells (treated) | 0 | 3.05 |
| Pancreas | 0 | 2.46 |
| Pancreatic ca. CAPAN 2 | 0 | 0.24 |
| Adipose | 6.22 | 6.65 |
| Adrenal gland | 0 | 2.48 |
| Thyroid | 0 | 2.77 |
| Salavary gland | 0 | 1.6 |
| Pituitary gland | 0 | 5.69 |
| Brain (fetal) | 0.29 | 2.01 |
| Brain (whole) | 8.51 | 18.56 |
| Brain (amygdala) | 2.1 | 2.26 |
| Brain (cerebellum) | 2.97 | 7.7 |
| Brain (hippocampus) | 0.08 | 2.36 |
| Brain (substantia nigra) | 0.9 | 8.04 |
| Brain (thalamus) | 0 | 1.74 |
| Brain (hypothalamus) | 0.08 | 0.88 |
| Spinal cord | 0 | 2.53 |
| CNS ca. (glio/astro) U87-MG | 0 | 0.15 |
| CNS ca. (glio/astro) U-118-MG | 0 | 3.07 |
| CNS ca. (astro) SW1783 | 0.03 | 1.28 |
| CNS ca.* (neuro; met) SK-N-AS | 0 | 14.52 |
| CNS ca. (astro) SF-539 | 0 | 1.11 |
| CNS ca. (astro) SNB-75 | 0.55 | 8.29 |
| CNS ca. (glio) SNB-19 | 0 | 1.03 |
| CNS ca. (glio) U251 | 0 | 1.76 |
| CNS ca. (gilo) SF-295 | 0 | 3.97 |
| Heart | 0 | 8.03 |
| Skeletal muscle | 0 | 3.63 |
| Bone marrow | 0 | 5.49 |
| Thymus | 1.24 | 7.21 |
| Spleen | 0 | 2.93 |
| Lymph node | 0 | 4.55 |
| Colon (ascending) | 0.08 | 4.38 |
| Stomach | 1.77 | 9.91 |
| Small intestine | 0 | 1.96 |
| Colon ca. SW480 | 0 | 0.13 |
| Colon ca.* (SW480 met)SW620 | 0 | 0.02 |
| Colon ca. HT29 | 0.02 | 0.47 |
| Colon ca. HCT-116 | 0.02 | 0.03 |
| Colon ca. CaCo-2 | 3.54 | 17.02 |
| Colon ca. HCT-15 | 0 | 0.52 |
| Colon ca. HCC-2998 | 0 | 1.07 |
| Gastric ca.* (liver met) NCI-N87 | 0 | 2.09 |
| Bladder | 9.14 | 10.86 |
| Trachea | 0 | 6.18 |
| Kidney | 0.02 | 1.91 |
| Kidney (fetal) | 6.8 | 10.51 |
| Renal ca. 786-0 | 0 | 0.51 |
| Renal ca. A498 | 0 | 1.04 |
| Renal ca. RXF 393 | 0 | 0.34 |
| Renal ca. ACHN | 0 | 2.75 |
| Renal ca. UO-31 | 0 | 0.78 |
| Renal ca. TK-10 | 0 | 0.68 |
| Liver | 0 | 3.33 |
| Liver (fetal) | 0 | 2.69 |
| Liver ca. (hepatoblast) HepG2 | 0 | 0.01 |
| Lung | 3.31 | 13.06 |
| Lung (fetal) | 0.01 | 2.85 |
| Lung ca. (small cell) LX-1 | 0 | 2.47 |
| Lung ca. (small cell) NCI-H69 | 2.4 | 7.48 |
| Lung ca. (s.cell var.) SHP-77 | 0 | 0.01 |
| Lung ca. (large cell) NCI-H460 | 0 | 0.02 |
| Lung ca. (non-sm. cell) A549 | 0 | 1.24 |
| Lung ca. (non-s.cell) NCI-H23 | 0 | 0.03 |
| Lung ca (non-s.cell) HOP-62 | 0 | 0.81 |
| Lung ca. (non-s.cl) NCI-H522 | 0 | 1.95 |
| Lung ca. (squam.) SW 900 | 0 | 5.94 |
| Lung ca. (squam.) NCI-H596 | 4.51 | 11.39 |
| Mammary gland | 21.71 | 8.01 |
| Breast ca.* (pl. effusion) MCF-7 | 0 | 0 |
| Breast ca.* (pl.ef) MDA-MB-231 | 0 | 0.01 |
| Breast ca.* (pl. effusion) T47D | 0 | 0.39 |
| Breast ca. BT-549 | 0.05 | 0.02 |
| Breast ca. MDA-N | 98.64 | 35.97 |
| Ovary | 0 | 1.27 |
| Ovarian ca. OVCAR-3 | 0 | 2.3 |
| Ovarian ca. OVCAR-4 | 0 | 7.56 |
| Ovarian ca. OVCAR-5 | 0 | 2.21 |
| Ovarian ca. OVCAR-8 | 0 | 3.52 |
| Ovarian ca. IGROV-1 | 0 | 1.45 |
| Ovarian ca.* (ascites) SK-OV-3 | 0 | 0.54 |
| Uterus | 0 | 3.32 |
| Plancenta | 0 | 1.38 |
| Prostate | 0 | 0.93 |
| Prostate ca.* (bone met)PC-3 | 0 | 0.02 |
| Testis | 0 | 2.91 |
| Melanoma Hs688(A).T | 0 | 0.09 |
| Melanoma* (met) Hs688(B).T | 0 | 0.71 |
| Melanoma UACC-62 | 0 | 9.44 |
| Melanoma M14 | 100 | 100 |
| Melanoma LOX IMVI | 0.02 | 11.75 |
| Melanoma* (met) SK-MEL-5 | 11.34 | 27.7 |
| Melanoma SK-MEL-28 | 18.61 | 35.23 |

From the results of the real-time quantitative PCR analysis (Table 28), it is concluded that clone 23208248 is highly expressed in a variety of melanomas and breast cancer tissues. Therefore, clone 23208248 or the protein encoded by it can be used to target therapies or imaging diagnostics to treat melanoma and breast cancer.

### EXAMPLE 6

### Expression of 10354784 in Human Embryonic Kidney 293 Cells

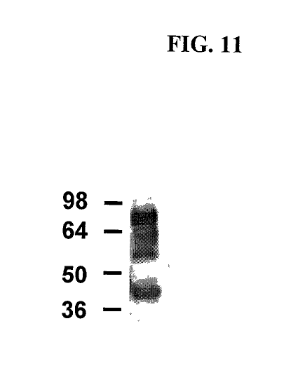

The BamHI-XhoI fragment containing a 10354784 coding sequence was isolated from CuraGen Corporation's clone pCR2.1-cg10354784 and subcloned into the vector pCEP4/Sec (Example AB2) to generate expression vector pCEP4/Sec-10354784. The pCEP4/Sec-10354784 vector was transfected into 293 cells using the LipofectaminePlus reagent following the manufacturer's instructions (Gibco/BRL). The cell pellet and supernatant were harvested 72 hours after transfection and examined for 10354784 expression by Western blotting (reducing conditions) with an anti-V5 antibody. FIG. 11 shows that proteins secreted by 293 cells include a V5 fusion of 10354784 expressed as multiple sized polypeptides with a main product having an apparent molecular weight of approximately 70 kDa. The remaining bands may represent proteolytic digestion products of the 70 kDa band.

### EXAMPLE 7

### Expression of 17883252 in Human Embryonic Kidney 293 Cells

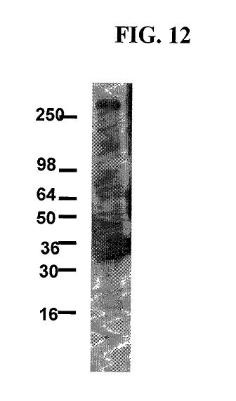

The BamHI-XhoI fragment containing a 17883252 coding sequence was isolated from CuraGen Corporation's clone pCR2.1-cg17883252 and subcloned into the vector pCEP4/Sec (Example AB2) to generate expression vector pCEP4/Sec-17883252. The pCEP4/Sec-17883252 vector was transfected into 293 cells using the LipofectaminePlus reagent following the manufacturer's instructions (Gibco/BRL). The cell pellet and supernatant were harvested 72 hours after transfection and examined for 17883252 expression by Western blotting (reducing conditions) with an anti-V5 antibody. It was found that 17883252 is expressed in the cell pellet as several polypeptides, with the principal product having an apparent molecular weight of approximately 36 kDa in 293 cells (FIG. 12).

### EXAMPLE 8

### Expression of 17941787 in Human Embryonic Kidney 293 Cells

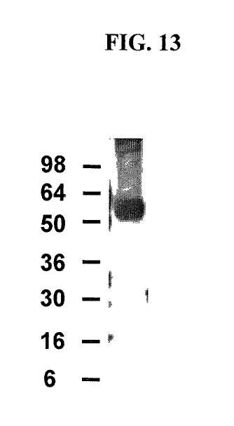

The KpnI-XhoI fragment containing a 17941787 coding sequence was isolated from CuraGen Corporation's clone 17941787-in pCR2.1 vector (whose designation includes the identifier S323-6c) and subcloned into KpnI-XhoI digested pCEP4/Sec (Example AB2) to generate expression vector pCEP4/Sec-17941787. The pCEP4/Sec-17941787 vector was transfected into 293 cells using the LipofectaminePlus reagent following the manufacturer's instructions (Gibco/BRL). The cell pellet and supernatant were harvested 72 hours after transfection and examined for 17941787 expression by Western blotting (reducing conditions) with an anti-V5 antibody. Expression of 17941787 by 293 cells was observed intracellularly as a polypeptide having an apparent molecular weight of approximately 55 kDa (FIG. 13).

Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated polypeptide comprising an amino acid sequence selected from the group consisting of:
(a) a mature form of an amino acid sequence selected from the group consisting of SEQ ID NO:2, 4, 6, 8, 10, 12, 14, 16, 18, 20, 22, 24, 26 and 62;
(b) a variant of a mature form of an amino acid sequence selected from the group consisting of SEQ ID NO:2, 4, 6, 8, 10, 12, 14, 16, 18, 20, 22, 24, and 26, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NO:2, 4, 6, 8, 10, 12, 14, 16, 18, 20, 22, 24, and 26; and
(d) a variant of an amino acid sequence selected from the group consisting of SEQ ID NO:2, 4, 6, 8, 10, 12, 14, 16, 18, 20, 22, 24, and 26, wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting of SEQ ID NO:2, 4, 6, 8, 10, 12, 14, 16, 18, 20, 22, 24, and 26.

3. The polypeptide of claim 2, wherein said allelic variant comprises an amino acid sequence that is the translation of a nucleic acid sequence differing by a single nucleotide from a nucleic acid sequence selected from the group consisting of SEQ ID NO:1, 3, 5, 7, 9, 11, 13, 15, 17, 19, 21, 23,and 25.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NO:2, 4, 6, 8, 10, 12, 14, 16, 18, 20, 22, 24, and 26;
(b) a variant of a mature form of an amino acid sequence selected from the group consisting of SEQ ID NO:2, 4, 6, 8, 10, 12, 14, 16, 18, 20, 22, 24, and 26, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NO:2, 4, 6, 8, 10, 12, 14, 16, 18, 20, 22, 24, and 26;
(d) a variant of an amino acid sequence selected from the group consisting of SEQ ID NO:2, 4, 6, 8, 10, 12, 14, 16, 18, 20, 22, 24, 26 and 62, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NO:2, 4, 6, 8, 10, 12, 14, 16, 18, 20, 22, 24, and 26, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NO:1, 3, 5, 7, 9, 11, 13, 15, 17, 19, 21, 23, and 25.

9. The nucleic acid molecule of claim 5, wherein said nucleic acid molecule comprises a nucleotide sequence selected from the group consisting of
(a) a nucleotide sequence selected from the group consisting of SEQ ID NO:1, 3, 5, 7, 9, 11, 13, 15, 17, 19, 21, 23, and 25;
(b) a nucleotide sequence differing by one or more nucleotides from a nucleotide sequence selected from the group consisting of SEQ ID NO:1, 3, 5, 7, 9, 11, 13, 15, 17, 19, 21, 23, and 25, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting of SEQ ID NO:1, 3, 5, 7, 9, 11, 13, 15, 17, 19, 21, 23, and 25, or a complement of said nucleotide sequence.

11. The nucleic acid molecule of claim 5, wherein the nucleic acid molecule comprises a nucleotide sequence selected from the group consisting of
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that immunospecifically-binds to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide,
thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. A method of identifying an agent that binds to a polypeptide of claim 1, the method comprising:
(a) contacting said polypeptide with said agent; and
(b) determining whether said agent binds to said polypeptide.

21. A method for identifying an agent that modulates the expression or activity of the polypeptide of claim 1, the method comprising:
(a) providing a cell expressing said polypeptide;
(b) contacting the cell with said agent; and
(c) determining whether the agent modulates expression or activity of said polypeptide,
whereby an alteration in expression or activity of said peptide indicates said agent modulates expression or activity of said polypeptide.

22. A method for modulating the activity of the polypeptide of claim 1, the method comprising contacting a cell sample expressing the polypeptide of said claim with a compound that binds to said polypeptide in an amount sufficient to modulate the activity of the polypeptide.

23. A method of treating or preventing a POLYX-associated disorder, said method comprising administering to a subject in which such treatment or prevention is desired the polypeptide of claim 1 in an amount sufficient to treat or prevent said POLYX-associated disorder in said subject.

24. The method of claim 23, wherein said subject is a human.

25. A method of treating or preventing a POLYX-associated disorder, said method comprising administering to a subject in which such treatment or prevention is desired the nucleic acid of claim 5 in an amount sufficient to treat or prevent said POLYX-associated disorder in said subject.

26. The method of claim 25, wherein said subject is a human.

27. A method of treating or preventing a POLYX-associated disorder, said method comprising administering to a subject in which such treatment or prevention is desired the antibody of claim 15 in an amount sufficient to treat or prevent said POLYX-associated disorder in said subject.

28. The method of claim 27, wherein the subject is a human.

29. A pharmaceutical composition comprising the polypeptide of claim 1 and a pharmaceutically-acceptable carrier.

30. A pharmaceutical composition comprising the nucleic acid molecule of claim 5 and a pharmaceutically-acceptable carrier.

31. A pharmaceutical composition comprising the antibody of claim 15 and a pharmaceutically-acceptable carrier.

32. A kit comprising in one or more containers, the pharmaceutical composition of claim 29.

33. A kit comprising in one or more containers, the pharmaceutical composition of claim 30.

34. A kit comprising in one or more containers, the pharmaceutical composition of claim 31.

35. The use of a therapeutic in the manufacture of a medicament for treating a syndrome associated with a human disease, the disease selected from a POLYX-associated disorder, wherein said therapeutic is selected from the group consisting of a POLYX polypeptide, a POLYX nucleic acid, and a POLYX antibody.

36. A method for screening for a modulator of activity or of latency or predisposition to a POLYX-associated disorder, said method comprising:
(a) administering a test compound to a test animal at increased risk for a POLYX-associated disorder, wherein said test animal recombinantly expresses the polypeptide of claim 1;
(b) measuring the activity of said polypeptide in said test animal after administering the compound of step (a);
(c) comparing the activity of said protein in said test animal with the activity of said polypeptide in a control animal not administered said polypeptide, wherein a change in the activity of said polypeptide in said test animal relative to said control animal indicates the test compound is a modulator of latency of or predisposition to a POLYX-associated disorder.

37. The method of claim 36, wherein said test animal is a recombinant test animal that expresses a test protein transgene or expresses said transgene under the control of a promoter at an increased level relative to a wild-type test animal, and wherein said promoter is not the native gene promoter of said transgene.

38. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease,
wherein an alteration in the expression level of the polypeptide in the first subject as compared to the control sample indicates the presence of or predisposition to said disease.

39. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

40. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NO:2, 4, 6, 8, 10, 12, 14, 16, 18, 20, 22, 24, and 26, or a biologically active fragment thereof.

41. A method of treating a pathological state in a mammal, the method comprising administering to the mammal the antibody of claim 15 in an amount sufficient to alleviate the pathological state.
